(19) United States
(12) Patent Application Publication
(10) Pub. No.: US 2023/0374589 A1
(43) Pub. Date: Nov. 23, 2023

(54) DETERMINING MORTALITY RISK OF SUBJECTS WITH VIRAL INFECTIONS

(71) Applicant: Inflammatix, Inc., Sunnyvale, CA (US)

(72) Inventors: Timothy Sweeney, Sunnyvale, CA (US); Ljubomir Buturovic, Sunnyvale, CA (US); Uros Midic, Sunnyvale, CA (US); Yudong He, Sunnyvale, CA (US)

(21) Appl. No.: 17/920,510

(22) PCT Filed: Apr. 29, 2021
(86) PCT No.: PCT/US2021/029847

§ 371 Date: Oct. 21, 2022

Related U.S. Application Data
(60) Provisional application No. 63/017,570, filed on Apr. 29, 2020.

(51) Int. Cl. C12Q 1/6883; G16H 50/30; G16H 50/20

(52) U.S. Cl. CPC C12Q 1/6883; G16H 50/30; G16H 50/20; C12Q 2600/158; C12Q 2600/118; G16H 50/80

(57) ABSTRACT

Systems, methods, compositions, apparatuses, and kits for determining the 30-day mortality risk of subjects with viral infections, and for determining effective triage strategies for such subjects, are provided herein. The disclosed methods and compositions involve biomarkers identified from the application of a machine learning workflow to viral mortality training data. The biomarkers allow the calculation of a score that can be used to determine the likelihood of 30-day survival in the subjects.

### CROSS REFERENCE TO RELATED APPLICATIONS

The present application claims priority to U.S. Provisional Pat. Appl. No. 63/017,570, filed on Apr. 29, 2020, which application is incorporated herein by reference in its entirety.

### BACKGROUND

The emergence of the SARS-coronavirus 2 (SARS-CoV-2), causative agent of COVID-19, and its rapid pandemic spread has led to a global health crisis with more than 54 million cases and more than 1 million deaths to date (1). COVID-19 presents with a spectrum of clinical phenotypes, with most patients exhibiting mild-to-moderate symptoms, and 20% progressing to severe or critical disease, typically within a week (2-6). Severe cases are often characterized by acute respiratory failure requiring mechanical ventilation and sometimes progressing to Acute Respiratory Distress Syndrome (ARDS) and death (7). Illness severity and development of ARDS are associated with older age and underlying medical conditions (3).

Yet, despite the rapid progress in developing diagnostics for SARS-CoV-2 infection, existing prognostic markers ranging from clinical data to biomarkers and immunopathological findings have proven unable to identify which patients are likely to progress to severe disease (8). Poor risk stratification means that front-line providers may be unable to determine which patients might be safe to quarantine and convalesce at home, and which need close monitoring. Early identification of severity along with monitoring of immune status may also prove important for selection of treatments such as corticosteroids, intravenous immunoglobulin, or selective cytokine blockade (9-11).

A host of lab values, including neutrophilia, lymphocyte counts, CD3 and CD4 T-cell counts, interleukin-6 and -8, lactate dehydrogenase, D-dimer, AST, prealbumin, creatinine, glucose, low-density lipoprotein, serum ferritin, and prothrombin time rather than viral factors have been associated with higher risk of severe disease and ARDS (3, 12, 13). While combining multiple weak markers through machine learning (ML) has a potential to increase test discrimination and clinical utility, applications of ML to date have led to serious overfitting and lack of clinical adoption (14). The failure of such models arises both from a lack of clinical heterogeneity in training, and from the pragmatic nature of the variable selection, which uses existing lab tests which may not be ideal for the task. Furthermore, a number of the lab markers are late indicators of severity since by the time they become abnormal, the patient is already very sick.

The host immune response represented in the whole blood transcriptome has been repeatedly shown to diagnose presence, type, and severity of infections (15-19). By leveraging clinical, biological, and technical heterogeneity across multiple independent datasets, we have previously identified a conserved host response to respiratory viral infections (16) that is distinct from bacterial infections (15-17) and can identify asymptomatic infection. This conserved host response to viral infections is strongly associated with severity of outcome (20). We have also demonstrated that conserved host immune response to infection can be an accurate prognostic marker of risk of 30-day mortality in patients with infectious diseases (18). Most importantly, we have demonstrated that accounting for biological, clinical, and technical heterogeneity identifies more generalizable robust host response-based signatures that can be rapidly translated on a targeted platform (19).

In the current COVID-19 pandemic, any future viral pandemic, or during seasonal influenza, there is a critical need for patient risk stratification at triage (for instance, in an emergency department) in order to preserve hospital resources for only those most in need. However, current biomarkers such as C-reactive protein and procalcitonin do not adequately risk stratify for effective triage. Accordingly, there is a need for new biomarkers that allow that rapid and accurate determination of risk, e.g., 30-day mortality risk, for patients with viral infections. The present disclosure satisfies this need and provides other advantages as well.

### BRIEF SUMMARY

In one aspect, the present disclosure provides a method of administering urgent care to a subject in an emergency room or other clinical facility with a diagnosis of a viral infection, the method comprising: (i) receiving a biological sample that was obtained from the subject; (ii) detecting expression levels of TGFBI, DEFA4, LY86, BATF and HK3 biomarkers in the biological sample; and (iii) determining a risk score based on the biomarker expression levels detected in step (ii), the score corresponding to a risk of mortality or of a need for ICU care of the subject over a specified length of time.

In some embodiments, the method further comprises. (iv) administering urgent care to the subject or discharging the subject from the emergency room or other clinical facility based on the risk score. In some embodiments of the method, the specified length of time is 30 days. In some embodiments, the method further comprises detecting the level of expression of an HLA-DPB1 biomarker in the biological sample in step (ii). In some embodiments, the score is compared to one or more thresholds corresponding to one or more discrete levels of risk of need for ICU care or mortality over 30 days. In some embodiments, the score is compared to two thresholds corresponding to a (i) low, (ii) intermediate, and (iii) high risk of need for ICU care or mortality over 30 days, allowing the subject to be classified into one of three risk categories corresponding to each level (i-iii) of risk.

In some embodiments, the risk score is also based on one or more clinical parameters determined for the subject. In some embodiments, the one or more clinical parameters comprises age or a clinical risk score. In some embodiments, the clinical risk score is a sequential organ failure assessment (SOFA) score. In some embodiments, the expression of the genes is detected using qRT-PCR or isothermal amplification. In some embodiments, the isothermal amplification method is qRT-LAMP. In some embodiments, the expression of the genes is detected using a NanoString nCounter. In some embodiments, the biological sample is a blood sample. In some embodiments, the diagnosis is based on a detection of viral antigen or viral nucleic acid in a biological sample taken from the subject. In some embodiments, the diagnosis is based on a detection of the expression levels of biomarkers associated with viral infection in a biological sample taken from the subject. In some embodiments, the expression levels of the biomarkers are detected within 24 hours of the diagnosis of viral infection.

In some embodiments, the threshold for a determination of a low risk of mortality or of a need for ICU care over 30 days corresponds to a likelihood ratio of less than 0.15. In some embodiments, the threshold for a determination of an intermediate risk of need for ICU care or mortality over 30 days corresponds to a likelihood ratio of from 0.15 to 5.

In some embodiments, the method further comprises discharging the subject from the emergency room or other clinical facility based on the risk score. In some such embodiments, the subject has been classified as having a low (i) risk of need for ICU care or mortality over 30 days. In some embodiments, the urgent care comprises administering organ-supportive therapy, administering a therapeutic drug, admitting the subject to an ICU, or administering a blood product. In some such embodiments, the subject has been classified as having an intermediate (ii) or high (iii) risk of need for ICU care or mortality over 30 days. In some embodiments, the organ-supportive therapy comprises connecting the subject to any one or more of a mechanical ventilator, a pacemaker, a defibrillator, a dialysis or a renal replacement therapy machine, or an invasive monitor selected from the group consisting of a pulmonary artery catheter, arterial blood pressure catheter, and central venous pressure catheter. In some embodiments, the therapeutic drug comprises an immune modulator, an antiviral agent, a coagulation modulator, a vasopressor, or a sedative. In some embodiments, the viral infection is an influenza or SARS-COV-2 infection.

In another aspect, the present disclosure provides a test kit for detecting the expression levels of five or more biomarkers in a subject with a viral infection, wherein the kit comprises reagents for specifically detecting the expression levels of the five or more biomarkers, and wherein the biomarkers comprise TGFBI, DEFA4, LY86, BATF and HK3. In some embodiments, the biomarkers further comprise HLA-DPB1. In some embodiments, the biomarkers comprise TGFBI, DEFA4, LY86, BATF, HK3, and HLA-DPB1.

In some embodiments, the kit comprises a microarray. In some embodiments, the kit comprises an oligonucleotide that hybridizes to TGFBI, an oligonucleotide that hybridizes to DEFA4, an oligonucleotide that hybridizes to LY86, an oligonucleotide that hybridizes to BATF, and an oligonucleotide that hybridizes to HK3. In some embodiments, the kit further comprises an oligonucleotide that hybridizes to HLA-DPB1. In some embodiments, the test kit further comprises one or more reagents, devices, containers, or implements for performing q-RT-PCR, qRT-LAMP, or NanoString nCounter analysis. In some embodiments, the viral infection is an influenza or SARS-CoV-2 infection. In some embodiments, the test kit further comprises instructions to calculate a mortality score based on the levels of expression of the biomarkers in the subject, the score corresponding to the risk of mortality of the subject over a specified length of time. In some embodiments, the specified length of time is 30 days. In some embodiments, the mortality score is further based on one or more clinical parameters established for the subject. In some embodiments, the one or more clinical parameters comprise age or a clinical risk score. In some embodiments, the clinical risk score is a SOFA score.

A better understanding of the nature and advantages of embodiments of the present disclosure may be gained with reference to the following detailed description and the accompanying drawings.

### BRIEF DESCRIPTION OF THE DRAWINGS

FIGS. 1A-1B. Two examples of 2-gene combinations out of the 15 selected genes, where (large) triangles are non-survival cases and (small) squares are survival cases.

FIGS. 2A-2D. Histogram of AUROCs obtained using (FIG. 2A) each of selected 15 genes, (FIG. 2B) 2-gene pairs of 15 selected genes, (FIG. 2C) a predictor consisting of 1, 2, and up to 15 ranked top 15 genes, and (FIG. 2D) each of the 13,902 genes.

FIGS. 3A-3B. FIG. 3A: Logistic regression model selection. Each dot corresponds to a model defined by logistic regression hyperparameters and a decision threshold (i.e., a threshold above which a score predicts 30-day mortality, and below which a score predicts 30-day survival). The entire search space (100 hyperparameter configurations) is shown. FIG. 3D: ROC plot for the best model. The plot is constructed using pooled probabilities from leave-one-study-out cross-validation folds.

FIG. 4. HostDx-ViralSeverity could be used both to rule out hospitalization for low-risk patients and to identify high-risk patients in need of hospitalization. Note that in this study only 10% of patients fall into a ‘moderate’/indeterminate band, meaning the test is useful in roughly 90% of cases, far more than either C-reactive protein or procalcitonin have shown in COVID-19.

FIG. 5. Multivariate model adjusted for age. The figure demonstrates that, even adjusted for age, the gene score remains significantly associated with mortality. That is, the score is a predictor of mortality independent of (even when corrected for) patient age.

FIG. 6. 5-mRNA risk score (‘viral_severity’) plotted against 30-day outcomes in the 41 patients with samples and clinical data available from the Athens COVID-19 cohort. Non-severe patients had no need for ICU or mechanical ventilation. The score showed a 96% sensitivity and 75% specificity for separating non-severe patients from severe and mortality patients.

FIG. 7: Distribution of single gene AUC. AUCs were calculated for predicting severe vs non-severe groups in the 62 patients. Shown are: AUC distribution using each of 15,788 genes detected (top, gray); AUCs using each of 150 down- (blue) or 329 up- (coral) regulated genes defined by absolute effect size >1.3, and p value <0.005; individual AUCs of 35 genes further selected for high expression and robust performance (green); and AUCs for all 2-gene combinations from 35 biomarker genes (purple).

FIG. 8. Biomarker selection based on frequency. The number of times each of top 46-ranked genes is present out of 62 leave-one-out (LOO) gene selections. Our selected 35 marker genes showed in at least 60 out of 62 LOOs with 33 showed in all 62 LOOs.

FIGS. 9A-9B. Performance of aggregated GM score to distinguish severe vs non-severe COVID-19 patients. Geometric mean score is based on geometric means of normalized expression of up (n=22) and down (n=13) differentially expressed genes. FIG. 9A: Boxplot of geometric mean score in non-severe (orange) and severe (blue) patients. FIG. 9B: ROC of the geometric means score.

FIGS. 10A-10B. Study flow. FIG. 10A: Clinical data flows for training and testing. FIG. 10B: Machine learning workflow used to develop and validate the 6-mRNA viral severity classifier. LOSO=Leave-One-Study-Out. CV=cross-validation. AUROC=Area Under ROC curve.

FIGS. 11A-11D. Training data for the 6-mRNA classifier. FIG. 11A: Visualization of 705 samples across 21 studies in low dimension using t-SNE. FIG. 11B: Logistic regression model selection. Each dot corresponds to a model defined by a combination of logistic regression hyperparameters and a decision threshold. Entire search space (100 hyperparameter configurations) is shown. FIG. 11C: ROC plot for the best model. The plot is constructed using pooled probabilities from cross-validation folds. FIG. 11D: Expression of the 6 genes used in the logistic regression model according to mortality outcomes.

FIGS. 12A-12D. Validation of the 6-mRNA classifier in the independent retrospective non-COVID-19 cohorts. FIG. 12A: Visualization of the samples using t-SNE. FIG. 12B: Expression of the 6 genes used in the logistic regression model in patients with clinically relevant subgroups. FIG. 12C: 6-mRNA classifier accurately distinguishes non-severe and severe patients with COVID-19 as well as those who died. FIG. 12D: ROC plot for the subgroups.

FIGS. 13A-13D. Validation of the 6-mRNA classifier in the COVID-19 cohort. FIG. 13A: Visualization of 97 samples in the prospective validation cohort using t-SNE. FIG. 13B: Expression of the 6 genes used in the logistic regression model in patients with severe and non-severe SARS-CoV-2 viral infection. FIG. 13C: 6-mRNA classifier accurately distinguishes non-severe and severe patients with COVID-19 as well as those who died. FIG. 13D: ROC plot for non-severe COVID-19 vs. severe or death (samples from healthy controls not included).

FIG. 14. Distribution of the pooled training set cross-validation 6-mRNA score for the best logistic regression model. Blue=survivors, red=non-survivors.

FIG. 15. Correlation of the 6-mRNA classifier scores using rapid qRT-LAMP panel and NanoString nCounter gold standard shows excellent agreement (Pearson R=0.95) across n=61 clinical samples.

FIG. 16 illustrates a measurement system 160 according to an embodiment of the present disclosure.

FIG. 17 shows a block diagram of an example computer system usable with systems and methods according to embodiments of the present disclosure.

### TERMS

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The term “nucleic acid” or “polynucleotide” refers to primers, probes, oligonucleotides, template RNA or cDNA, genomic DNA, amplified subsequences of biomarker genes, or any polynucleotide composed of deoxyribonucleic acids (DNA), ribonucleic acids (RNA), or any other type of polynucleotide which is an N-glycoside of a purine or pyrimidine base, or modified purine or pyrimidine bases in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). “Nucleic acid”, “DNA” “polynucleotides, and similar terms also include nucleic acid analogs. The polynucleotides are not necessarily physically derived from any existing or natural sequence, but can be generated in any manner, including chemical synthesis, DNA replication, reverse transcription or a combination thereof.

“Primer” as used herein refers to an oligonucleotide, whether occurring naturally or produced synthetically, that is capable of acting as a point of initiation of synthesis when placed under conditions in which synthesis of a primer extension product which is complementary to a nucleic acid strand is induced i.e., in the presence of nucleotides and an agent for polymerization such as DNA polymerase and at a suitable temperature and buffer. Such conditions include the presence of four different deoxyribonucleoside triphosphates and a polymerization-inducing agent such as DNA polymerase or reverse transcriptase, in a suitable buffer (“buffer” includes substituents which are cofactors, or which affect pH, ionic strength, etc.), and at a suitable temperature. The primer is preferably single-stranded for maximum efficiency in amplification such as a TaqMan real-time quantitative RT-PCR as described herein. The primers herein are selected to be substantially complementary to the different strands of each specific sequence to be amplified, and a given set of primers will act together to amplify a subsequence of the corresponding biomarker gene.

The term “gene” refers to the segment of DNA involved in producing a polypeptide chain. It can include regions preceding and following the coding region (leader and trailer) as well as intervening sequences (introns) between individual coding segments (exons).

SARS-CoV-2 refers to the coronavirus that causes the infectious disease called COVID-19. The present methods can be used to determine the 30-day mortality risk (or risk of other outcomes such as intensive care unit (ICU) admission, secondary infections, or mortality at other time points such as 7, 14, 60 days, etc.) of any subject with any viral infection and including any SARS-CoV-2 infection, including by infection with viruses comprising the nucleotide sequences of, or comprising nucleotide sequences substantially identical (e.g., 70%, 75%, 80%, 85%, 90%, 95% or more identical) to all or a portion of GenBank reference numbers MN908947, LC757995, LC528232, or another SARS-CoV-2 genome. The methods can be performed with subjects having an infection detected by any method, and regardless of the presence or absence of symptoms.

As used herein, a “biomarker gene” or “biomarker” refers to a gene whose expression is correlated with a mortality or other outcome in a subject with a viral infection, e.g., survival or non-survival, ICU admission, secondary infection, etc. at, e.g., 3, 7, 14, 28, 30, 60, or 90 days, in a subject with, e.g., influenza or SARS-CoV-2. The expression level of each of the genes need not be correlated with the mortality rate in all patients; rather, a correlation will exist at the population level, such that the level of expression is sufficiently correlated within the overall population of individuals with a viral infection and with a known 30-day mortality outcome, that it can be combined with the expression levels of other biomarker genes, in any of a number of ways, as described elsewhere herein, and used to calculate a biomarker or mortality score. The values used for the measured expression level of the individual biomarker genes can be determined in any of a number of ways, including direct readouts from relevant instruments or assay systems, or values determined using methods including, but not limited to, forms of linear or non-linear transformation, rescaling, normalizing, z-scores, ratios against a common reference value, or any other means known to those of skill in the art. In some embodiments, the readout values of the biomarkers are compared to the readout value of a reference or control, e.g., a housekeeping gene whose expression is measured at the same time as the biomarkers. For example, the ratio or log ratio of the biomarkers to the reference gene can be determined. Preferred biomarker genes for the purposes of the present methods include TGFBI, DEFA4, LY86, BATF and HK3, or TGFBI, DEFA4, LY86, BATF, HK3, and HLA-DPB1, but others can be used as well, e.g., other biomarkers identified using the machine learning methods described herein.

A “biomarker score”, “mortality score”, or “risk score”, terms which can be used interchangeably, refers to a value allowing a determination of the probability of mortality (or other outcome) in a subject with a viral infection that is calculated from the measured expression levels of a plurality of biomarker genes, e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or more individual biomarker genes, in the subject. In some embodiments, the risk score is determined by applying a mathematical formula, or a series of mathematical formulae with specified interconnections, or a machine learning algorithm with optimized hyperparameters, or another parameter-based method by which the measured expression values of the biomarker genes can be used to generate a single “risk” score, including, e.g., arithmetic or geometric means with or without weights, linear regression, logistic regression, neural nets, or any other method known in the art. In particular embodiments, the “risk score” is used to determine the 30-day mortality risk (or need for ICU care) of a subject, by virtue of the score surpassing or not a given threshold value for the outcome in question, as described in more detail elsewhere herein. The risk score (or a different risk score, obtained using a different mathematical formula, algorithm, etc., as described herein) can also be used to determine or predict other aspects of infection-related risk in the subject, such as the length of hospital stay, the need for ICU care, the rate of readmission of the subject, etc. The risk score can also be combined with one or more clinical parameters, alone or in combination, such as age, comorbidity status, or a risk score such as qSOFA, SOFA, APACHE, or others known in the art, e.g., to improve the performance of the score in determining risk of mortality or other outcome.

The term “correlating” generally refers to determining a relationship between one random variable with another. In various embodiments, correlating a given biomarker level or score with the presence or absence of a condition or outcome (e.g., survival or non-survival at 30 days) comprises determining the presence, absence or amount of at least one biomarker in a subject with the same outcome. In specific embodiments, a set of biomarker levels, absences or presences is correlated to a particular outcome, using receiver operating characteristic (ROC) curves.

“Conservatively modified variants” refers to nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

One of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. In some cases, conservatively modified variants can have an increased stability, assembly, or activity.

As used in herein, the terms “identical” or percent “identity,” in the context of describing two or more polynucleotide sequences, refer to two or more sequences or specified subsequences that are the same. Two sequences that are “substantially identical” have at least 60% identity, preferably 65%, 70%, 75%, 80%, 85%, 90%, 91%, 92%, 93, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. The identity can exists over a region that is at least about 10, 15, 20, 25, 30, 35, 40, 45, 50, or more nucleotides in length. In some embodiments, percent identity is determined over the full-length of the nucleic acid sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm with, e.g., the default parameters can be used. See, e.g., Altschul et al., (1990) J. Mol. Biol. 215: 403410 and the National Center for Biotechnology Information website, ncbi.nlm.nih.gov.

### DETAILED DESCRIPTION

The present disclosure provides methods and compositions for estimating the 30-day (or other time period) mortality risk or risk of severe disease in subjects with viral infections, and for determining effective triage strategies for such subjects, e.g., when present in an emergency room setting. The present methods and compositions involve biomarkers identified from the application of a machine learning workflow to viral mortality training data, i.e., expression data from patients with known viral infections and known 30-day outcomes (survival or non-survival). Using these data, biomarkers have been identified that allow the calculation of a score that can be used to determine the likelihood of 30-day survival (or need for intensive care) in subjects with a diagnosis of a viral infection, e.g., infection with SARS-CoV-2 or influenza.

### I. SUBJECTS

The present methods and compositions can be used to determine a risk score (e.g., a 30-day mortality or need for intensive care unit (ICU) care score) for subjects having a viral infection. In various embodiments, the subject may be an adult, a child, or an adolescent. The subject may be male or female.

The subject has received a diagnosis of a viral infection, e.g., influenza or SAR-CoV-2. The diagnosis can be made directly, e.g., by detection of viral genomic sequences, e.g., by RT-PCR, or by detection of antibodies against the virus, e.g., by ELISA. In some embodiments, the diagnosis is made indirectly. e.g., by a clinical assessment of the subject's symptoms and/or known exposure to the virus. In some embodiments, the diagnosis is made by assessing biomarkers associated with viral infection, e.g., as described in Sweeney et al., (2016) Sci. Transl. Med., 8 (346): 346ra91; and WO2017214061, the entire disclosures of which are herein incorporated by reference.

In particular embodiments, the subject is present in an emergency care context, e.g., emergency room, urgent care facility, hospital, or any other clinical setting where diagnosis may take place. A clinical setting does not necessarily indicate that the patient is physically present in a hospital or clinical facility, however. For example, the patient may be at home but has received a diagnosis, e.g., through a remote consultation with a medical professional, using an at-home testing kit, or through a local or drive-up testing facility. The results of the methods described herein can allow a determination of the optimal next step or plan of action for the subject's care. For example, a determination that the subject has a low risk of 30-day mortality can indicate that, for a subject presenting in an emergency room, that they can be discharged from the hospital or emergency room, e.g., to return home for monitoring or to go to another, non-emergency ward. A subject with a high risk of 30-day mortality can be sent, e.g., to the ICU and/or administered any of another of subsequent treatment options, as described in more detail elsewhere herein. Any course of action taken in view of an intermediate or high risk score, including admittance to an ICU or administration of any of the treatments described herein, are considered “urgent care” for the purposes of the present disclosure.

The present methods provide a more specific approach with respect to viral infections than our previous work concerning mortality risk (see, e.g., U.S. Pat. No. 10,344,332, Sweeney et al., (2018) Nature Commun. 15(9):694). This earlier work showed that host response can accurately predict outcomes such as those described in paragraph [030] in all comers. However, the underlying host immune response differs according to the physiologic insult, e.g., between bacterial infections, viral infections, and non-infectious inflammation. While our prior risk score was designed as an all-comers risk score, the present disclosure provides a risk score that is specifically designed for use only in patients with viral infections, and as such allows for improved risk stratification in these patients and, in some cases, the use of fewer biomarkers.

The present methods can be used to determine the 30-day mortality risk caused by any virus, e.g., influenza, coronavirus, Ebolavirus, Marburg, hantavirus, rotavirus. SARS coronavirus, MERS coronavirus, adenovirus, adeno-associated virus, aichi virus, alphapapillomavirus, alphavirus, alphacoronavirus, alphatorquevirus, arenavirus, Australian bat lyssavirus, BK polyomavirus, Banna virus, Barmah forest virus, betacoronavirus, Bunyamwera virus, Bunyavirus La Crosse, Bunyavirus snowshoe hare, cardiovirus, Cercopithecine herpesvirus, Chandipura virus, Chikungunya virus, Cosavirus, cosavirus, Cowpox virus, Coxsackievirus, Crimean-Congo cytomegalovirus, hemorrhagic fever virus, deltavirus, deltaretrovirus, Dengue virus, dependovirus. Dhori virus, Dugbe virus, Duvenhage virus, eastern equine encephalitis virus, echovirus, encephalomvocarditis virus, enterovirus, Epstein-Barr virus, erythrovirus, European bat lyssavirus, flavivirus, GB virus C/Hepatitis G virus, Hantaan virus, hantavirus, henipavirus, Hendra virus, henipavirus, Hepatitis A, B, C. E, or delta virus, hepatovirus, hepacivirus, hepevirus, Horsepox virus, astrovirus, cytomegalovirus, enterovirus, herpesvirus, HIV, kobuvirus, lyssavirus, papillomavirus, parainfluenza, parvovirus, respiratory syncytial virus, rhinovirus, spumaretrovirus, T-lymphotropic virus, torovirus, Isfahan virus, JC polyomavirus. Japanese encephalitis virus, Junin arenavirus, KI Polymavirus, Kunjin virus, Lagos bat virus, Lak Victoria Marburgvirus, Langat virus, Lassa virus, lentivirus, Lordsdale virus, Louping ill virus, lymphocryptovirus, Lymphocytic choriomeningitis virus, lyssavirus, Machupo virus, Marburgvirus, mastadenovirus, mamastrovirus, Mayaro virus, measles virus, mengo encephalomyocarditis virus, Merkel cell polyomavirus. Mokola virus, molluscipoxvirus, Molluscum contagiosum virus, monkeypox virus, mumps virus, mupapillomavirus, Murray valley encephalitis virus, nairovirus, New York virus, Nipah virus, norovirus. Norwalk virus, O'nvong-nyong virus, Orf virus, Oropouche virus, orthobynyavirus, orthohepadnavirus, orthopneumovirus, orthopoxvirus, hepacivirus, orthopoxvirus, pegivirus, Pichinde virus, poliovirus, poly omavirus, Punta toro phlebovirus, Puumala virus, rabies virus, respirovirus, rhadinovirus, Rift valley fever virus, Rosavirus, roseolovirus, Ross river virus, rotavirus, rubella virus, rubulavirus, sagiyama virus, salivirus A, sandfly fever Sicilian virus, sapovirus, Sapporo virus, seadornavirus, semliki forest virus, Seoul virus, simian foamy virus, simian virus, simplexvirus, sindbis virus, Southampton virus, spumavirus, St. Louis encephalitis virus, thogotovirus, tick-bome powassan virus, torque teno virus, torovirus, Toscana virus, Uukuniemi virus, vaccinia virus, varicella-zoster virus, varicellovirus, variola virus, Venezuelan equine encephalitis virus, vesicular stomatitis virus, vesiculovirus, western equine encephalitis virus, WU polyomavirus, West Nile virus, Yaba monkey tumor virus, Yaba-like disease virus, Yellow fever virus, Zika virus, and others. In particular embodiments, the subject has a coronavirus, e.g., SARS-CoV-2, or influenza. The subject can be infected during a pandemic, epidemic, seasonal, or isolated infection incident. In particular embodiments, the infection is detected in the context of an epidemic or pandemic, i.e., when health care resources are limited and rapid triage of subjects presenting in emergency care contexts is critical.

### II. BIOLOGICAL SAMPLES

To assess the biomarker status of the patient, a biological sample is obtained from the subject, e.g. a blood sample is taken by a phlebotomist, in a way that allows the mRNA to be collected and preserved. In some embodiments, a blood sample is collected directly into a tube prefilled with a solution that can immediately stabilize RNA from blood cells within the sample. One suitable tube is the PAXgene Blood RNA Tube (QIAGEN, BD cat. No. 762165), although any tube capable of preserving RNA can be used. A non-RNA preserving tube such as a K2-EDTA tube can also be used, provided that it is tested within a certain amount of time after venipuncture (e.g., within 15, 30, 60, or 120 minutes), or is kept cold, or both. Biomarker polynucleotides that are poorly expressed in particular cells may be enriched using normalization techniques (Bonaldo et al., 1996, Genome Res. 6:791-806). In particular embodiments, the sample is taken within 24 hours of the initial diagnosis of viral infection.

Typically, the biological sample comprises whole blood, buffy coat, plasma, serum, or blood cells such as peripheral blood mononuclear cells (PBMCS), T cells, mature, immature or developing leukocytes, including lymphocytes, polymorphonuclear leukocytes, neutrophils, monocytes, reticulocytes, basophils, band cells, metamelocytes, coelomocytes, hemocytes, eosinophils, megakaryocytes, macrophages, dendritic cells, natural killer cells, or fraction of such cells (e.g., a nucleic acid or protein fraction). Other biological samples that can be used for the purposes of the present methods, including, inter alia, saliva, urine, sweat, nasal swab, nasopharyngeal swab, rectal swab, ascitic fluid, peritoneal fluid, synovial fluid, amniotic fluid, cerebrospinal fluid, and tissue biopsy. The biological sample can be obtained from the subject by conventional techniques, e.g., venipuncture for blood samples or surgical techniques for solid tissue samples.

### III. SELECTION OF BIOMARKERS

The 30-day mortality risk of a subject with a diagnosis of a viral infection is determined by calculating a score (e.g., “biomarker score” or “mortality score”) based on the expression levels of biomarkers. In some embodiments, a panel of five biomarkers is used to calculate the score. In particular embodiments, the biomarker genes are TGFBI, DEFA4, LY86, BATF and HK3. In some embodiments, a panel of six biomarkers is used to calculate the score. In particular embodiments, the biomarker genes are TGFBI, DEFA4, LY86, BATF, HK3, and HLA-DPB1. TGFBI refers to transforming growth factor beta induced (see, e.g., NCBI gene ID 7045, the entire disclosure of which is herein incorporated by reference). DEFA4 refers to defensin alpha 4 (see, e.g., NCBI gene ID 1669, the entire disclosure of which is herein incorporated by reference). LY86 refers to lymphocyte antigen 86 (see, e.g., NCBI gene ID 9450, the entire disclosure of which is herein incorporated by reference). BATF refers to basic leucine zipper ATF-like transcription factor (see, e.g., NCBI gene ID 10538, the entire disclosure of which is herein incorporated by reference), HK3 refers to hexokinase 3 (see., e.g., NCBI gene ID 3101, the entire disclosure of which is herein incorporated by reference), and HLA-DPB1 refers to major histocompatibility complex class II DP beta 1 (see, e.g., NCBI gene ID 3115, the entire disclosure of which is herein incorporated by reference).

However, other biomarkers can be used, e.g., in place of or in addition to TGFBI, DEFA4, LY86, BATF, and HK3, or TGFBI, DEFA4, LY86, BATF, HK3, and HLA-DPB1. For example, in some embodiments, other biomarkers used in the methods include, but are not limited to, TDRD1, POLE, MYOM1, PDZD4, HHLA3, PDE4B, HSPA14, PRDM2, TSPANI3, GAB4, RPL4, EGLN1, TRIM67, AACS, and ST8SIA3. Any number of biomarkers can be assessed in the methods, e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30 or more biomarkers. Other biomarkers that can be used include those disclosed in, e.g., Mayhew et al. (2020) Nature Commun. 11, Art. 1177; Sweeney et al., (2018) Nature Commun. 9(1):694: Sweeney et al. (2015) Sci. Transl. Med. 7(287):287ra71; Sweeney et al., (2016) Sci. Transl. Med. 8(346):346ra91; Sweeney et al., (2018) Crit. Care Med. 46(6):915-925, and patent publications WO2016145426, WO2017214061, WO201916822, and WO2018004806, the entire disclosures of each of which is herein incorporated by reference. In some embodiments, the biomarkers comprise any one or more of the genes listed in Table 1. In some embodiments, the biomarkers comprise any one or more of the genes listed in Table 5. In some embodiments, the biomarkers comprise any one or more of the gene pairs listed in Table 3. In some embodiments, the biomarkers comprise any one or more of the gene pairs listed in Table 6.

The biomarkers used in the present methods correspond to genes whose expression levels correlate with 30-day mortality (or other) outcomes in subjects having a viral infection, e.g., SARS-CoV-2 or influenza. It will be appreciated that the expression level of the individual biomarkers can be elevated or depressed relative to the level in survivors or non-survivors with the same viral infection. What is important is that the expression level of the biomarker is positively or inversely correlated with survival or non-survival, allowing the determination of an overall score. e.g., a risk score, or biomarker score or mortality score, that can be used to determine the 30-day mortality risk for a subject, e.g., a low, intermediate, or high risk of 30-day mortality.

Additional biomarkers can be assessed and identified using any standard analysis method or metric, e.g., by analyzing data from samples taken from subjects with a diagnosis of a viral infection and with a known 30-day outcome (i.e., 30-day survival or non-survival), as described in more detail elsewhere herein and as illustrated, e.g., in the Examples. In particular methods, the types of viral infections of the training data include that of the subject, but this is not required. Suitable metrics and methods include Pearson correlation, Kendall rank correlation, Spearman rank correlation, t-test, other non-parametric measures, over-sampling of the non-survival group, under-sampling of the survival group, and others including linear regression, non-linear regression, random forest and other tree-based methods, artificial neural networks, etc. In a particular embodiment, the feature selection uses univariate ranking with the absolute value of the Pearson correlation between the gene expression and outcome as the ranking metric. In some embodiments, features (genes) are selected via greedy forward search optimized on training accuracy. In some embodiments, features (genes) are selected via greedy forward search optimized on Area Under Operator Receiver Characteristic.

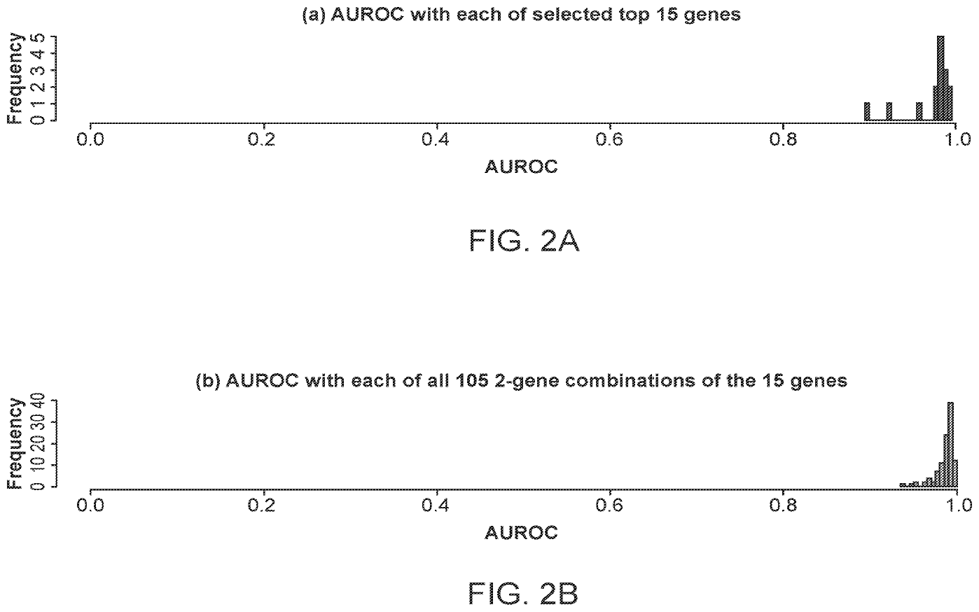

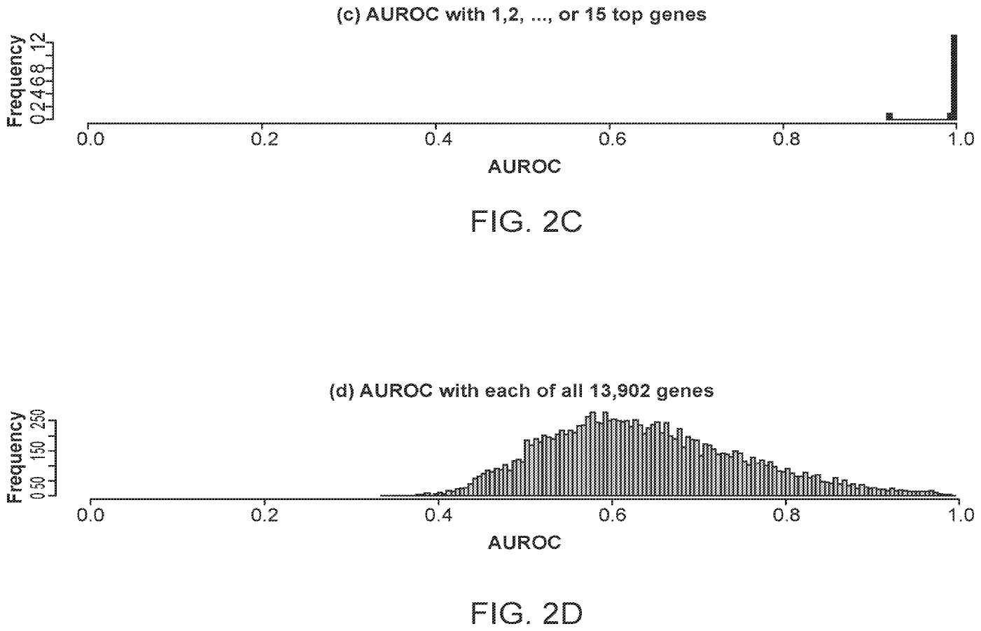

In particular embodiments, a machine learning workflow is applied to the training data, e.g., using a separate validation set or using cross-validation. For example, hyperparameter tuning can be used over a search space of parameters, e.g., parameters known to be effective for model optimization for infectious disease diagnosis. Examples of classifiers that can be used include linear classifiers such as Support Vector Machine with linear kernel, logistic regression, and multi-layer perceptron with linear activation function. Feature selection can be performed using the gene expression data for the candidate biomarkers as independent variables and using the known outcome as the dependent variable. The different models can be evaluated, e.g., using plots based on sensitivity and false-positive rates for each model, and the decision threshold evaluated during the hyperparameter search, and using ROC-like plots based on pooled cross-validated probabilities for the best models. (See, e.g., Ramkumar et al., Development of a Novel Proteomic Risk-Classifier for Prognostication of Patients with Early-Stage Hormone Receptor-Positive Breast Cancer. Biomarker Insights, Vol. 13, 1-9, 2018, FIG. 2A). Any of a number of different variants of cross-validation (CV) can be used, such as 5-fold random CV, 5-fold grouped CV, where each fold comprises multiple studies, and each study is assigned to exactly one CV fold, and leave-one-study-out (LOSO), where each study forms a CV fold. In some embodiments, the number of genes included in the final model can be limited, e.g., to 5 or 6, to facilitate translation to a rapid molecular assay. For example, the number of genes can be reduced by selecting those genes with the highest levels of expression.

### IV. DETECTING BIOMARKER EXPRESSION

As described in more detail below, data sets corresponding to the biomarker gene expression levels as described herein are used to create a diagnostic or predictive rule or model based on the application of a statistical and machine learning algorithm, in order to produce a mortality risk score. Such an algorithm uses relationships between a biomarker profile and an outcome, e.g., survival and non-survival at 30 days (sometimes referred to as training data). The data are used to infer relationships that are then used to predict the status of a subject, e.g. the risk of mortality at 30 days.

The expression levels of the biomarkers can be assessed in any of a number of ways. In particular embodiments, the expression levels of the biomarkers are determined by measuring polynucleotide levels of the biomarkers. For example, once blood or another biological sample has been collected and preserved, RNA can be extracted using any method, so long that it permits the preservation of the RNA for subsequent quantification of the expression levels of the biomarker genes and of any control genes to be used, e.g., housekeeping genes used as reference values for the biomarkers. RNA can be extracted, e.g., from preserved blood cells manually, or using a robotic apparatus, such as Qiacube (QIAGEN) with a commercial RNA extraction kit. In some embodiments, RNA extraction is not performed, e.g., for isothermal amplification methods. In such methods, expression levels can be determined directly through lysis of, e.g., blood cells, and then, e.g., reverse transcription and amplification of mRNA.

In some embodiments, the reference nucleic acid is a housekeeping gene or a product thereof, such as a corresponding mRNA transcript. In some embodiments, the reference nucleic acid includes an mRNA transcript that is a pre-mRNA molecule, a 5′ capped mRNA molecule, a 3′ adenylated mRNA molecule, or a mature mRNA molecule. In particular embodiments, the reference nucleic acid is a mature mRNA molecule obtained from a mammalian host that is also the source of the test sample. In some embodiments, the housekeeping gene or product thereof is expressed at a relatively constant rate by a cell of the host, such that the expression rate of the housekeeping gene can be used as a reference point against the expression of other host genes or gene products thereof. Suitable housekeeping genes are well known in the art and may include, e.g., GAPDH, ubiquitin, 18S (18S rRNA, e.g., HGNC (Human Genome Nomenclature Committee) nos. 44278-44281, 37657). ACTB (Actin beta, e.g., HGNC no. 132)), KPNA6 (Karyopherin subunit alpha 6, e.g., HGNC no. 6399), or RREB1 (ras-responsive element binding protein 1, e.g., HGNC no. 10449).

In some embodiments, the reference nucleic acid is a human housekeeping gene. Exemplary human housekeeping genes suitable for use with the present methods include, but are not limited to, KPNA6, RREB1, YWHAB, Chromosome 1 open reading frame 43 (Clorf43), Charged multivesicular body protein 2A (CHMP2A), ER membrane protein complex subunit 7 (EWC7), Glucose-6-phosphate isomerase (GPI), Proteasome subunit, beta type, 2 (PSMB2), Proteasome subunit, beta type, 4 (PSMB4), Member RAS oncogene family (RAB74). Receptor accessory protein 5 (REEPS), small nuclear ribonucleoprotein D3 (SNRPD3), Valosin containing protein (VCP) and vacuolar protein sorting 29 homolog (VPS29). In some embodiments, any housekeeping gene provided at www/tau % ac/il˜elieis/HKG/may be used (see, Eisenberg and Levanon., Trends Genel. (2013), 10:569-74).

The levels of transcripts of the biomarker genes, or their levels relative to one another, and/or their levels relative to a reference gene such as a housekeeping gene, can be determined from the amount of mRNA, or polynucleotides derived therefrom, present in a biological sample. Polynucleotides can be detected and quantified by a variety of methods including, but not limited to, NanoString (e.g., nCounter analysis), microarray analysis, polymerase chain reaction (PCR), reverse transcriptase polymerase chain reaction (RT-PCR), quantitative RT-PCR (qRT-PCR), serial analysis of gene expression (SAGE), isothermal amplification methods such as qRT-LAMP, internal DNA detection switch, northern blotting, RNA fingerprinting, ligase chain reaction, Qbeta replicase, strand displacement amplification, transcription based amplification systems, nuclease protection (Si nuclease or RNAse protection assays), sequencing methods, as well as methods disclosed in International Publication Nos. WO 88/10315 and WO 89/06700, and International Applications Nos. PCT/US87/00880 and PCT/US89/01025; herein incorporated by reference in their entireties, and methods using MacMan probes, flip probes, and TaqMan probes (see, e.g., Murray et al. (2014) J. Mol Diag. 16:6, pp 627-638). See, e.g., Draghici, Data Analysis Tools for DNA Microarrays, Chapman and Hall/CRC, 2003: Simon et al., Design and Analysis of DNA Microarray Investigations, Springer, 2004; Real-Time PCR: Current Technology and Applications, Logan, Edwards, and Saunders eds., Caister Academic Press, 2009; Bustin, A-Z of Quantitative PCR (IUL Biotechnology, No. 5), International University Line, 2004; Velculescu et al. (1995) Science 270: 484-487; Matsumura et al. (2005) Cell. Microbiol. 7: 11-18; Serial Analysis of Gene Expression (SAGE): Methods and Protocols (Methods in Molecular Biology), Humana Press, 2008; each of which is herein incorporated by reference in its entirety.

In some embodiments, the biomarker gene expression is detected using a gene expression panel such as a NanoString nCounter, which allows the quantification of biomarker gene expression without the need for amplification or cDNA conversion. In such methods, RNA obtained from the blood or other biological sample from the subject is hybridized in solution to probes, e.g., a labeled reporter probe and a capture probe for each biomarker and control sequence. The target RNA-probe complexes are then purified and immobilized on a solid support, and then quantified, with each marker-specific probe having a specific fluorescent signature that allows the quantification of the specific marker. Such methods and the generation of probes, e.g., capture probes and reporter probes, for such applications are known in the art and are described, e.g., on the website nanostring.com.

For amplification-based methods such as qRT-PCR or qRT-LAMP, the primers can be obtained in any of a number of ways. For example, primers can be synthesized in the laboratory using an oligo synthesizer, e.g., as sold by Applied Biosvstems. Biolytic Lab Performance, Sierra Biosystems, or others. Alternatively, primers and probes with any desired sequence and/or modification can be readily ordered from any of a large number of suppliers, e.g., ThermoFisher, Biolytic, IDT, Sigma-Aldritch, GeneScript, etc.

Computer programs that are well known in the art are useful in the design of primers with the required specificity and optimal amplification properties, such as Oligo version 5.0 (National Biosciences). PCR methods are well known in the art, and are described, for example, in Innis et al., eds., PCR Protocols: A Guide To Methods And Applications. Academic Press Inc., San Diego, Calif. (1990): herein incorporated by reference in its entirety.

In some embodiments, microarrays are used to measure the levels of biomarkers. An advantage of microarray analysis is that the expression of each of the biomarkers can be measured simultaneously, and microarrays can be specifically designed to provide a diagnostic expression profile for a particular disease or condition (e.g., influenza, SARS-CoV-2, etc.). Microarrays are prepared by selecting probes which comprise a polynucleotide sequence, and then immobilizing such probes to a solid support or surface. For example, the microarray may comprise a support or surface with an ordered array of binding (e.g., hybridization) sites or “probes” each representing one of the biomarkers described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). Each probe is preferably covalently attached to the solid support at a single site. Conditions for preparing microarrays, for hybridization conditions, and for detection of bound probes are well known in the art (see, e.g., Sambrook, et al., Molecular Cloning: A Laboratory Manual (3rd Edition, 2001); Ausubel et al., Current Protocols In Molecular Biology, vol. 2, Current Protocols Publishing, New York (1994); Shalon et al., 1996, Genome Research 6:639-645; Schena et al., Genome Res. 6:639-645 (1996); and Ferguson et al., Nature Biotech. 14:1681-1684 (1996)).

As noted above, the “probe” to which a particular polynucleotide molecule specifically hybridizes contains a complementary polynucleotide sequence. The probes of the microarray typically consist of nucleotide sequences of, e.g., no more than 1,000 nucleotides, or of 10 to 1,000 nucleotides or 10-200, 10-30, 10-40, 20-50, 40-80, 50-150, or 80-120 nucleotides in length. The probes may comprise DNA sequences, RNA sequences, or copolymer sequences of DNA and RNA. The polynucleotide sequences of the probes may also comprise DNA and/or RNA analogs, derivatives, or combinations thereof. For example, the probes can be modified at the base moiety, at the sugar moiety, or at the phosphate backbone (e.g., phosphorothioates). The polynucleotide sequences of the probes may be synthesized nucleotide sequences, such as synthetic oligonucleotide sequences. The probe sequences can be synthesized either enzymatically in vivo, enzymatically in vitro (e.g., by PCR), or non-enzymatically in vitro.

Probes are preferably selected using an algorithm that takes into account binding energies, base composition, sequence complexity, cross-hybridization binding energies, and secondary structure. See Friend et al., International Patent Publication WO 01/05935, published Jan. 25, 2001: Hughes et al., Nat. Biotech. 19:342-7 (2001). An array will include both positive control probes, e.g., probes known to be complementary and hybridizable to sequences in the target polynucleotide molecules, and negative control probes, e.g., probes known to not be complementary and hybridizable to sequences in the target polynucleotide molecules. In addition, the present methods will include probes to both the biomarkers themselves, as well as to internal control sequences such as housekeeping genes, as described in more detail elsewhere herein.

In one embodiment, a microarray is provided comprising an oligonucleotide that hybridizes to a TGFBI polynucleotide, an oligonucleotide that hybridizes to a DEFA4 polynucleotide, an oligonucleotide that hybridizes to a LY86 polynucleotide, an oligonucleotide that hybridizes to a BATF polynucleotide, and an oligonucleotide that hybridizes to an HK3 polynucleotide. In one embodiment, the disclosure provides a microarray comprising an oligonucleotides that hybridize to a TGFBI polynucleotide, an oligonucleotide that hybridizes to a DEFA4 polynucleotide, an oligonucleotide that hybridizes to a LY86 polynucleotide, an oligonucleotide that hybridizes to a BATF polynucleotide, an oligonucleotide that hybridizes to an HK3 polynucleotide, and an oligonucleotide that hybridizes to an HLA-DPB1 polynucleotide. In some embodiments, the disclosure provides a microarray comprising an oligonucleotide that hybridizes to any of the biomarkers listed in Table 1 or Table 5. In some embodiments, the disclosure provides a microarray comprising two oligonucleotides that hybridize to any of the biomarker pairs listed in Table 3 or Table 6.

In some embodiments, quantitative reverse transcriptase PCR (qRT-PCR) is used to determine the expression profiles of biomarkers (see, e.g., U.S. Patent Application Publication No. 2005/0048542A1: herein incorporated by reference in its entirety). The first step in gene expression profiling by RT-PCR is the reverse transcription of the RNA template into cDNA, followed by its exponential amplification in a PCR reaction. The two most commonly used reverse transcriptases are avilo mveloblastosis virus reverse transcriptase (AMV-RT) and Moloney murine leukemia virus reverse transcriptase (MLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

In some embodiments, the PCR employs the Taq DNA polymerase, which has a 5′-3′ nuclease activity but lacks a 3′-5′ proofreading endonuclease activity. TAQMAN PCR typically utilizes the 5′-nuclease activity of Taq or Tth polymerase to hydrolyze a hybridization probe bound to its target amplicon, but any enzyme with equivalent 5′ nuclease activity can be used. In such methods, two oligonucleotide primers are used to generate an amplicon typical of a PCR reaction, and a third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN RT-PCR can be performed using commercially available equipment, such as, for example, ABI PRISM 7700 sequence detection system. (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700 sequence detection system. The system consists of a thermocycler, laser, charge-coupled device (CCD), camera and computer. The system includes software for running the instrument and for analyzing the data. 5′-Nuclease assay data are initially expressed as Ct, or the threshold cycle. Fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs that can be used to normalize patterns of gene expression include mRNAs for the housekeeping genes glyceraldehyde-3-phosphate-dehydrogenase (GAPDH) and beta-actin.

In particular embodiments, the biomarker gene expression is determined using isothermal amplification. Isothermal amplification is a process in which a target nucleic acid is amplified using a constant, single, amplification temperature (e.g., from about 30° C. to about 95° C.). Unlike standard PCR, an isothermal amplification reaction does not include multiple cycles of denaturation, hybridization, and extension, of an annealed oligonucleotide to form a population of amplified target nucleic molecules (i.e., amplicons). There are various types of isothermal application known in the art, including but not limited to, loop-mediated isothermal amplification (LAMP), nucleic acid sequence based amplification NASBA, recombinase polymerase amplification (RPA), rolling circle amplification (RCA), nicking enzyme amplification reaction (NEAR), and helicase dependent amplification (HDA).

In particular embodiments, the isothermal amplification is real-time quantitative isothermal amplification, in which a target nucleic acid is amplified at a constant temperature and the target nucleic acid rate of amplification is monitored by fluorescence, turbidity, or similar measures (e.g,. NEAR or LAMP). In some cases, RNA (e.g., mRNA) is isolated from a biological sample and is used as a template to synthesize cDNA by reverse-transcription. cDNA molecules are amplified under isothermal amplification conditions such that the production of amplified target nucleic acid can be detected and quantitated.

In particular embodiments, the isothermal amplification is Loop-Mediated Isothermal Amplification (LAMP). LAMP offers selectivity and employs a polymerase and a set of specially designed primers that recognize distinct sequences in the target nucleic acid (see, e.g., Nixon et al., (2014) Bimolecular Detection and Quantitation, 2:4-10; Schuler et al., (2016) Anal Methods., 8:2750-2755; and Schoepp et al., (2017) Sci. Transl. Med., 9:eaal3693). Unlike PCR, the target nucleic acid is amplified at a constant temperature (e.g., 60-65° C.) using multiple inner and outer primers and a polymerase having strand displacement activity. In some instances, an inner primer pair containing a nucleic acid sequence complementary to a portion of the sense and antisense strands of the target nucleic acid initiate LAMP. Following strand displacement synthesis by the inner primers, strand displacement synthesis primed by an outer primer pair can cause release of a single-stranded amplicon. The single-stranded amplicon may serve as a template for further synthesis primed by a second inner and second outer primer that hybridize to the other end of the target nucleic acid and produce a stem-loop nucleic acid structure. In subsequent LAMP cycling, one inner primer hybridizes to the loop on the product and initiates displacement and target nucleic acid synthesis, yielding the original stem-loop product and a new stem-loop product with a stem twice as long. Additionally, the Y terminus of an amplicon loop structure serves as initiation site for self-templating strand synthesis, yielding a hairpin-like amplicon that forms an additional loop structure to prime subsequent rounds of self-templated amplification. The amplification continues with accumulation of many copies of the target nucleic acid. The final products of the LAMP process are stem-loop nucleic acids with concatenated repeats of the target nucleic acid in cauliflower-like structures with multiple loops formed by annealing between alternately inverted repeats of a target nucleic acid sequence in the same strand.

In some embodiments, the isothermal amplification assay comprises a digital reverse-transcription loop-mediated isothermal amplification (dRT-LAMP) reaction for quantifying the target nucleic acid (see, e.g., Khorosheva et al., (2016) Nucleic Acid Research, 44:2 e10). Typically, LAMP assays produce a detectable signal (e.g., fluorescence) during the amplification reaction. In some embodiments, fluorescence can be detected and quantified. Any suitable method for detecting and quantifying florescence can be used. In some instances, a device such as Applied Biosystem's QuantStudio can be used to detect and quantify fluorescence from the isothermal amplification assay.

Any suitable method for detecting amplification of a target nucleic acid in a test sample by quantitative real-time isothermal amplification may be used to practice the present methods. In some embodiments, quantitative real-time isothermal amplification of a target nucleic acid in a test sample is determined by detecting of one or more different (distinct) fluorescent labels attached to nucleotides or nucleotide analogs incorporated during isothermal amplification of the target nucleic acid (e.g., 5-FAM (522 nm), ROX (608 nm), FITC (518 nm) and Nile Red (628 nm). In another embodiment, quantitative real-time isothermal amplification of a target nucleic acid in a test sample can be determined by detection of a single fluorophore species (e.g., ROX (608 nm)) attached to nucleotides or nucleotide analogs incorporated during isothermal amplification of the target nucleic acid. In some embodiments, each fluorophore species used emits a fluorescent signal that is distinct from any other fluorophore species, such that each fluorophore can be readily detected among other fluorophore species present in the assay.

In some embodiments, methods of detecting amplification of a target nucleic acid in a test sample by quantitative real-time isothermal amplification can include using intercalating fluorescent dyes, such as SYTO dyes (SYTO 9 or SYTO 82). In some embodiments, methods of detecting amplification of a target nucleic acid in a test sample by quantitative real-time isothermal amplification can include using unlabeled primers to isothermally amplify the target nucleic acid in the test sample, and a labeled probe (e.g., having a fluorophore) to detect isothermal amplification of the target nucleic acid in the test sample. In some embodiments, unlabeled primers are used to isothermally amplify a target nucleic acid present in the test sample, and a probe is used having a 5-FAM dye label on the 5′ end and a minor groove binder (MGB) and non-fluorescent quencher on the 3′ end to detect isothermal amplification of the target nucleic acid (e.g., TaqMan Gene Expression Assays from ThermoFisher Scientific).

In some embodiments, detecting amplification of the target nucleic acid in the test sample is performed using a one-step, or two-step, quantitative real-time isothermal amplification assay. In a one-step quantitative real-time isothermal amplification assay, reverse transcription is combined with quantitative isothermal amplification to form a single quantitative real-time isothermal amplification assay. A one-step assay reduces the number of hands-on manipulations as well as the total time to process a test sample. A two-step assay comprises a first-step, where reverse transcription is performed, followed by a second-step, where quantitative isothermal amplification is performed. It is within the scope of the skilled artisan to determine whether a one-step or two-step assay should be performed.

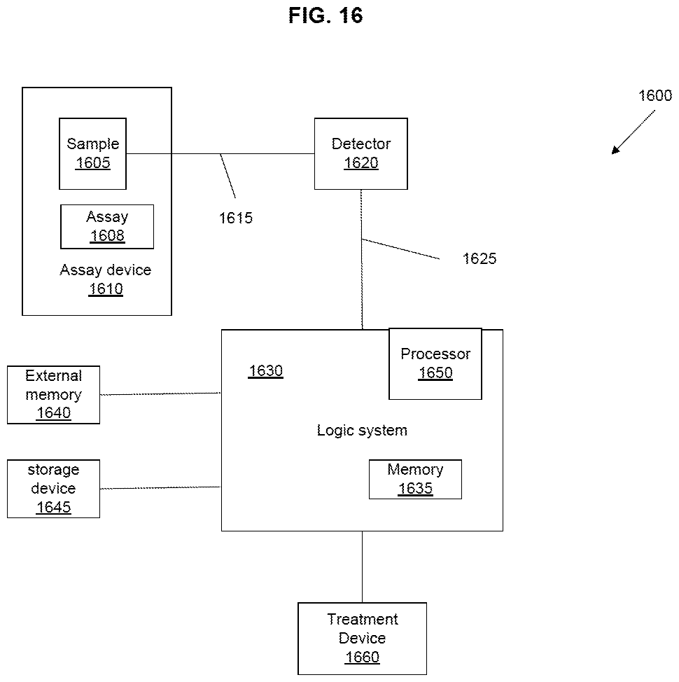

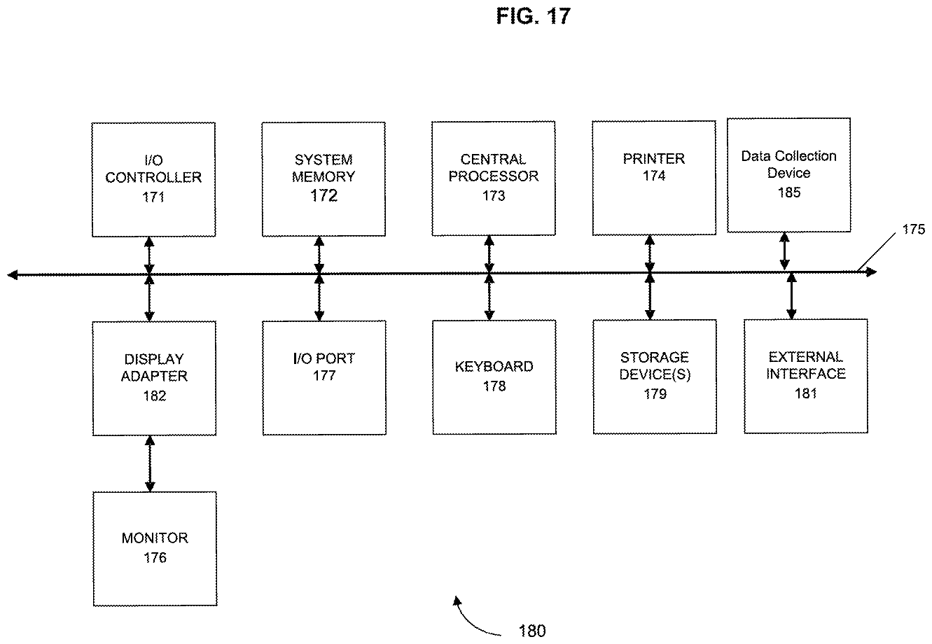

In some embodiments, the amplification and/or detection is carried out in whole or in part using an integrated measurement system, as illustrated in FIG. 16, which may also comprise a computer system as described elsewhere herein (see, e.g., FIG. 17).

In some embodiments, the risk or biomarker scores are calculated based on the Tt (time to threshold) values for each of the tested biomarkers. This may be accomplished by, e.g., establishing standard curves for the isothermal or other amplification of the target nucleic acid (e.g., biomarker) and the reference nucleic acid (e.g., housekeeping gene). The standard curves can be obtained by performing real-time isothermal amplification assays using quantitated calibrator samples with multiple known input concentrations. Appropriate methods are provided in, e.g., PCT Publication No. WO 2020/061217, the entire disclosure of which is herein incorporated by reference.

For example, in some embodiments, to generate a standard curve, quantitated calibrator samples are obtained by performing serial dilutions of a quantitated material. For example, a template is serially diluted in a buffer at 10-fold concentration intervals yielding templates covering a range of concentrations from, e.g., approximately 10⁹ copies/μl to approximately 10² copies/μL. The precise concentration of each calibrator sample can be determined using methods known in the art.

To obtain a standard curve, a real-time amplification assay is performed for each aliquot with a known quantity (e.g., 1 μL) of a respective calibrator sample with a respective concentration of the target nucleic acid. In a real-time amplification assay for each respective calibrator sample, the intensity of the fluorescence emitted by intercalating fluorescent dyes (e.g., dsDNA dyes) or fluorescent labels for the target nucleic acid is measured as a function of time. For example, a plot can be generated of fluorescence intensity as a function of time in a real-time quantitative amplification assay. A dashed line can be used to represent a pre-determined threshold intensity, and the elapsed time from the moment when the amplification is started is the time-to-threshold T. A respective time-to-threshold value can be determined from each respective fluorescence curve as a function of time. Thus, time-to-threshold values Ttₙ, Ttₙ₊₁, Ttₙ₊₂, etc., are obtained for the different calibrator samples.

For exponential amplifications, the time-to-threshold is linearly proportional to the logarithm (e.g., logarithm to base 10) of the starting copy number (also referred to as template abundance). A scatter plot of data points can be generated from the fluorescence curves. Each data point represents a data pair [Log₁₀(CopyNumber), Tt] (note that CopyNumber refers to starting number of copies of a nucleic acid in an amplification assay). In some embodiments, the data points fall approximately on a straight line. A linear regression is then performed on the data points in the plot to obtain the straight line that best fits the data points with the least amount of total deviations. The result of the linear regression is a straight line represented by the following equation,

Tt=m×Log₁₀(CopyNumber)÷b, (1)

where m is the slope of the line, and b is y-intercept. The slope m represents the efficiency of the isothermal amplification of the target nucleic acid; b represents a time-to-threshold as template copy number approaches zero. The straight line represented by Equation (1) is referred to as the standard curve.

In some embodiments, replicates (e.g., triplicates) of isothermal amplification assays may be run for each sample in order to gain a higher level of confidence in the data. Replicate time-to-threshold values can be averaged, and standard deviations can be calculated.

Once the standard curve is established for a given isothermal amplification assay, the standard curve can be used to convert a time-to-threshold value to a starting copy number for future runs of the amplification assay of unknown starting numbers of copies of the target nucleic acid, using the following equation,

$$CopyNumber = {10}^{\frac{Tt - b}{m} .} \qquad ( 2 )$$

Normally, the data points for low copy numbers or very high copy numbers may fall off of the straight line. The range of copy numbers within which the data points can be represented by the straight line is referred to as the dynamic range of the standard curve. The linear relationship between the time-to-threshold and the logarithmic of copy number represented by the standard curve would be valid only within the dynamic range.

If the amplification efficiencies for a target nucleic acid and a reference nucleic acid are different for a given isothermal amplification assay, it may be necessary to obtain separate standard curves for the target nucleic acid and the reference nucleic acid. Thus, two sets of real-time isothermal amplification assays may be performed, one set for establishing the standard curve for the target nucleic acid, the other set for establishing the standard curve for the reference nucleic acid. In cases where multiple target nucleic acids are considered (e.g., for a panel of five biomarkers as described herein), a standard curve for each target nucleic acid may be obtained.

In some embodiments, the standard curves are generated prior to obtaining a test sample. That is, the standard curves are not generated on-board with the quantitative isothermal amplification of the test sample. Such standard curves may be referred to as off-board standard curves. Off-board standard curves may be used for estimating relative abundance values. For example, for a test sample of unknown input concentration of a target nucleic acid, a first real-time amplification assay is performed for a first aliquot of the test sample to obtain a first time-to-threshold value with respect to the target nucleic acid. A second real-time isothermal amplification assay is then performed for a second aliquot of the test sample to obtain a second time-to-threshold value with respect to a reference nucleic acid. The first aliquot and the second aliquot contain substantially the same amount of the test sample. The first time-to-threshold value may then be converted into starting number of copies of the target nucleic acid using the standard curve of the target nucleic acid. Similarly, the second time-to-threshold value may be converted into starting number of copies of the reference nucleic acid using the standard curve of the reference nucleic. The starting number of copies of the target nucleic acid is then normalized against that of the reference nucleic acid to obtain a relative abundance value.

In cases where the amplification efficiencies for a target nucleic acid and a reference nucleic acid have approximately the same value that is known, relative abundance may be obtained directly from time-to-threshold values without using standard curves.

### V. CALCULATING BIOMARKER SCORES

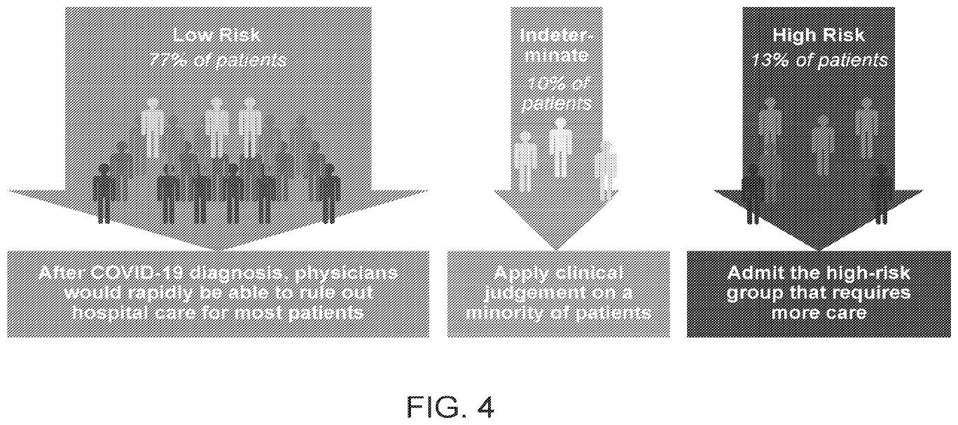

To determine the mortality risk, e.g., the risk at 30 days, a model (e.g., the model with the hyperparameter configuration providing the maximum AUC) is applied to the biomarker expression data from the subject to determine a score, e.g., a “risk score”. “biomarker score”, “mortality score”, “30-day mortality score”, or “HostDx-Viral Severity score”, that is indicative of the probability of mortality, e.g., the mortality at 30 days or at another time point, the risk of ICU admission, etc. This score can be used, e.g., to classify the subject into any of a number of bins, e.g., 3 bins with a “low”, “intermediate” or “indeterminate”, and “high” risk of mortality (see, e.g., FIG. 4). In a particular embodiment, the model uses logistic regression and the selected biomarker genes, e.g., TGFBI, DEFA4, LY86. BATF and HK3, or TGFBI, DEFA4, LY86, BATF, HK3, and HLA-DPB1 to calculate the score. The probability of mortality at 30 days as determined using the model is then used to determine the optimal treatment of the subject, as described in more detail elsewhere herein.,

The risk or biomarker score can be calculated, e.g., by taking the sum, product, or quotient of the gene levels, taken in terms of their absolute levels or their relative levels as compared to control genes, e.g., housekeeping genes, or by inputting them into a linear or nonlinear algorithm that incorporates at least the measured gene levels, e.g., the measured levels of 2, 3, 4, 5, 6, 7, 8, 9, 10 or more biomarker genes, into an interpretable score. In a particular embodiment, the score is calculated based on the expression data obtained for a panel of five biomarkers. In a particular embodiment, the score is calculated based on the expression data obtained for a panel of six biomarkers.

In semi-quantitative methods, a threshold or cut-off value is suitably determined, and is optionally a predetermined value. In particular embodiments, the threshold value is predetermined in the sense that it is fixed, for example, based on previous experience with the assay and/or a population of subjects with a given outcome or outcomes, e.g., with a population of 50, 100, 150, 200, 250, 300, 350, 400, 450, 500, or more subjects with survival or non-survival outcomes at 30 days. Alternatively, the predetermined value can also indicate that the method of arriving at the threshold is predetermined or fixed even if the particular value vanes among assays or can even be determined for every assay run.

For the statistical analyses described herein, e.g., for the selection of biomarkers to be included in the calculation of a score or in the calculation of a probability or likelihood of a particular mortality risk in a patient, as well as for diagnostic or therapeutic assessments made in view of a given risk or biomarker score, other relevant information can also be considered, such as clinical data regarding one or more conditions suffered by each individual. This can include demographic information such as age, race, and sex; information regarding a presence, absence, degree, stage, severity or progression of a condition, clinical risk scores such as SOFA, qSOFA, or APACHE, phenotypic information, such as details of phenotypic traits, genetic or genetically regulated information, amino acid or nucleotide related genomics information, results of other tests including imaging, biochemical and hematological assays, other physiological scores, or the like.

As described above, the abundance values for the individual biomarker genes can be combined using a mathematical formula or a machine learning or other algorithm to produce a single diagnostic score, such as the mortality score that can predict the 30 day mortality risk of a subject. In these embodiments, the produced score carries more predictive power than any individual gene level alone (e.g., has a greater area under the receiver-operating-characteristic curve for discrimination of survival or non-survival at 30 days).

In some embodiments, types of algorithms for integrating multiple biomarkers into a single diagnostic score may include, but not limited to, a difference of geometric means, a difference of arithmetic means, a difference of sums, a simple sum, and the like. In some embodiments, a diagnostic score may be estimated based on the relative abundance values of multiple biomarkers using machine-learning models, such as a regression model, a tree-based machine-learning model, a support vector machine (SVM) model, an artificial neural network (ANN) model, or the like.

Biomarker data may also be analyzed by a variety of methods to determine the statistical significance of differences in observed levels of biomarkers between test and reference expression profiles in order to evaluate the mortality risk for a subject within 30 days. In certain embodiments, patient data is analyzed by one or more methods including, but not limited to, multivariate linear discriminant analysis (LDA), receiver operating characteristic (ROC) analysis, principal component analysis (PCA), ensemble data mining methods, significance analysis of microarrays (SAM), cell specific significance analysis of microarrays (csSAM), spanning-tree progression analysis of density-normalized events (SPADE), and multi-dimensional protein identification technology (MUDPIT) analysis. (See, e.g., Hilbe (2009) Logistic Regression Models, Chapman & Hall/CRC Press; McLachlan (2004) Discriminant Analysis and Statistical Pattem Recognition. Wiley Interscience; Zweig et al. (1993) Clin. Chem. 39:561-577; Pepe (2003) The statistical evaluation of medical tests for classification and prediction, New York, N.Y.: Oxford; Sing et al. (2005) Bioinformatics 21:3940-3941; Tusher et al. (2001) Proc. Natl. Acad. Sci. U.S.A. 98:5116-5121; Oza (2006) Ensemble data mining, NASA Ames Research Center, Moffett Field, Calif. USA; English et al. (2009) J. Biomed. Inform. 42(2):287-295: Zhang (2007) Bioinformatics 8: 230: Shen-Orr et al. (2010) Journal of Immunology 184:144-130; Qiu et al. (2011) Nat. Biotechnol. 29(10):886-891; Ru et al. (2006) J. Chromatogr. A 1111(2):166-174, Jolliffe Principal Component Analysis (Springer Series in Statistics. 2.sup.nd edition, Springer, N Y, 2002). Koren et al. (2004) IEEE Trans Vis Comput Graph 10:459-470; herein incorporated by reference in their entireties.)

It is not necessary that all of the biomarkers are elevated or depressed relative to control levels in a given subject to give rise to a determination of a 30-day mortality or probability. For example, for a given biomarker level there can be some overlap between individuals falling into different probability categories. However, collectively the combined levels for all of the biomarker genes included in the assay will give rise to a score that, if it surpasses a threshold, e.g., a threshold derived from at least 50, 100, 150, 200, 250, 300, 350, 400, 500 or more patients with a viral infection and a survivor outcome, and/or of 10, 20, 30, 40, 50, 100, 150, 200, 250, 300, 350, 400, 500 or more control individuals with a viral infection and a non-survivor outcome, that allows a determination concerning the 30-day mortality risk of the subject. For example, for a determination of a low risk of mortality at 30 days, the threshold could be such that at across a population of at least 100 individuals with a viral infection and a 30-day survivor outcome and 100 patients with a viral infection and a non-survivor outcome, at least 90% of the subjects alive at 30 days are above the threshold. It will be appreciated that in any given assay there can be more than one threshold, e.g., a threshold in one direction that indicates a high risk of mortality, and a threshold in the other direction that indicates a low risk of mortality.

As used herein, the terms “probability,” and “risk” with respect to a given outcome refer to conditional probability that subjects with a particular score actually have the condition (e.g., 30 day non-survival) based on a given mathematical model. An increased probability or risk for example can be relative or absolute and can be expressed qualitatively or quantitatively. For instance, an increased risk can be expressed as simply determining the subject's score and placing the test subject in an “increased risk” category, based upon previous population studies. Alternatively, a numerical expression of the test subject's increased risk can be determined based upon an analysis of the biomarker or risk score.

In some embodiments, likelihood is assessed by comparing the level of a biomarker or mortality score to one or more preselected or threshold levels. Threshold values can be selected that provide an acceptable ability to predict risk of 30 day mortality, or of one or more aspects of care such as hospital length of stay, need for ICU care, need for mechanical ventilation, rate of readmission, etc. In illustrative examples, receiver operating characteristic (ROC) curves are calculated by plotting the value of a biomarker or risk score in two populations in which a first population has a first condition (e.g., non-survival at 30 days) and a second population has a second condition (e.g., non-survival at 30 days).

For any particular biomarker, a distribution of biomarker levels for subjects with and without a disease will likely overlap, and some overlap will be present for biomarker or risk scores as well. Under such conditions, a test does not absolutely distinguish a first condition and a second condition with 100% accuracy, and the area of overlap indicates where the test cannot distinguish the first condition and the second condition. A threshold value is selected, above which (or below which, depending on how a biomarker or risk score changes with a specified condition or prognosis) the test is considered to be “positive” and below which the test is considered to be “negative.” The area under the ROC curve (AUC) provides the C-statistic, which is a measure of the probability that the perceived measurement will allow correct identification of a condition (see, e.g., Hanley et al., Radiology 143: 29-36 (1982)).

In some embodiments, a positive likelihood ratio, negative likelihood ratio, odds ratio, and/or AUC or receiver operating characteristic (ROC) values are used as a measure of a method's ability to predict the mortality risk. As used herein, the term “likelihood ratio” is the probability that a given test result would be observed in a subject with a condition or outcome of interest divided by the probability that that same result would be observed in a patient without the condition or outcome of interest. Thus, a positive likelihood ratio is the probability of a positive result observed in subjects with the specified condition or outcome divided by the probability of a positive results in subjects without the specified condition or outcome. A negative likelihood ratio is the probability of a negative result in subjects without the specified condition or outcome divided by the probability of a negative result in subjects with specified condition or outcome.

The term “odds ratio,” as used herein, refers to the ratio of the odds of an event occurring in one group (e.g., a survivor at 30 days group) to the odds of it occurring in another group (e.g., a non-survivor at 30 days group), or to a data-based estimate of that ratio. The term “area under the curve” or “AUC” refers to the area under the curve of a receiver operating characteristic (ROC) curve, both of which are well known in the art. AUC measures are useful for evaluating the accuracy of a classifier across the complete decision threshold range. Classifiers with a greater AUC have a greater capacity to classify unknowns correctly between two or more groups of interest (e.g., a low, intermediate, or high risk of mortality at 30 days). ROC curves are useful for plotting the performance of a particular feature (e.g., any of the biomarker expression levels or biomarker scores described herein and/or any item of additional biomedical information) in distinguishing or discriminating between two populations (e.g., survivors or non-survivors). Typically, the feature data across the entire population (e.g., the cases and controls) are sorted in ascending order based on the value of a single feature. Then, for each value for that feature, the true positive and false positive rates for the data are calculated. The sensitivity is determined by counting the number of cases above the value for that feature and then dividing by the total number of cases. The specificity is determined by counting the number of controls below the value for that feature and then dividing by the total number of controls.

Although this refers to scenarios in which a feature is elevated in cases compared to controls, it also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to produce a single value, and this single value can be plotted in a ROC curve. Additionally, any combination of multiple features, in which the combination derives a single output value, can be plotted in a ROC curve. These combinations of features can comprise a test. The ROC curve is the plot of the sensitivity of a test against I-specificity of the test, where sensitivity is traditionally presented on the vertical axis and 1-specificity is traditionally presented on the horizontal axis. Thus, “AUC ROC values” are equal to the probability that a classifier will rank a randomly chosen positive instance higher than a randomly chosen negative one.

In some embodiments, at least two (e.g., 2, 3, 4, 5, 6, 7, 8, 9, 10, or more) biomarker genes are selected to discriminate between subjects with a first condition or outcome and subjects with a second condition or outcome with at least about 70%, 75%, 80%, 85%, 90%. 95% accuracy or having a C-statistic of at least about 0.70, 0.75, 0.80, 0.85, 0.90, 0.95.

In the case of a positive likelihood ratio, a value of 1 indicates that a positive result is equally likely among subjects in both the “condition” and “control” groups (e.g., in non-survivors and survivors at 30 days): a value greater than 1 indicates that a positive result is more likely in the condition group (e.g., in non-survivors); and a value less than 1 indicates that a positive result is more likely in the control group (e.g., in survivors). In this context, “condition” is meant to refer to a group having one characteristic (e.g., non-survival at 30 days) and “control” group lacking the same characteristic (e.g., survival at 30 days). In the case of a negative likelihood ratio, a value of 1 indicates that a negative result is equally likely among subjects in both the “condition” and “control” groups; a value greater than 1 indicates that a negative result is more likely in the “condition” group; and a value less than 1 indicates that a negative result is more likely in the “control” group.

In certain embodiments, the biomarker or risk score is calculated, based on the measured levels of the biomarkers in subjects with a viral infection and a 30-day survivor outcome or a viral infection and a 30-day non-survivor outcome, such that the likelihood ratio corresponding to the high risk bin is 1.5, 2, 2.5, 3, 3.5, 4, or more, or that the likelihood ratio corresponding to the low risk bin is 0, 15, 0.10, 0.05, or lower, for mortality at 30 days or for need for ICU care.

In the case of an odds ratio, a value of 1 indicates that a positive result is equally likely among subjects in both the condition” and “control” groups: a value greater than 1 indicates that a positive result is more likely in the “condition” group; and a value less than 1 indicates that a positive result is more likely in the “control” group. In the case of an AUC ROC value, this is computed by numerical integration of the ROC curve. The range of this value can be 0.5 to 1.0. A value of 0.5 indicates that a classifier (e.g., a biomarker level) cannot discriminate between cases and controls (e.g., non-survivors and survivors), while 1.0 indicates perfect diagnostic accuracy. In certain embodiments, biomarker gene levels and/or biomarker scores are selected to exhibit a positive or negative likelihood ratio of at least about 1.5 or more or about 0.67 or less, at least about 2 or more or about 0.5 or less, at least about 5 or more or about 0.2 or less, at least about 10 or more or about 0.1 or less, or at least about 20 or more or about 0.05 or less.

In certain embodiments, the biomarker gene levels and/or biomarker scores are selected to exhibit an odds ratio of at least about 2 or more or about 0.5 or less, at least about 3 or more or about 0.33 or less, at least about 4 or more or about 0.25 or less, at least about 5 or more or about 0.2 or less, or at least about 10 or more or about 0.1 or less. In certain embodiments, biomarker gene levels and/or biomarker scores are selected to exhibit an AUC ROC value of greater than 0.5, preferably at least 0.6, more preferably 0.7, still more preferably at least 0.8, even more preferably at least 0.9, and most preferably at least 0.95.

In some cases, multiple thresholds can be determined in so-called “tertile.” “quartile,” or “quintile” analyses. In these methods, the “diseased” and “control groups” (or “high risk” and “low risk”) groups are considered together as a single population, and are divided into 3, 4, or 5 (or more) “bins” having equal numbers of individuals. The boundary between two of these “bins” can be considered “thresholds.” A risk (of a particular diagnosis or prognosis for example) can be assigned based on which “bin” a test subject falls into. In particular embodiments, subjects are assigned to one of three bins, i.e. “low”. “intermediate”, or “high”, referring to the risk of 30-day mortality or risk of need for ICU care based on the risk scores obtained using the present methods. For example, subjects can be classified according to the estimated probability of death at 30 days into 3 bins: low likelihood (bin 1), intermediate (bin 2), and high-likelihood (bin 3). The bins are defined, e.g., such that the likelihood ratios are <0.15 in bin 1, from 0.15 to 5 in bin 2, and >5 in bin 3.

The phrases “assessing the likelihood” and “determining the likelihood,” as used herein, refer to methods by which the skilled artisan can predict the presence or absence of a condition (e.g., of survival or non-survival at 30 days) in a patient. The skilled artisan will understand that this phrase includes within its scope an increased probability that a condition is present or absent in a patient; that is, that a condition is more likely to be present or absent in a subject. For example, the probability that an individual identified as having a specified condition actually has the condition can be expressed as a “positive predictive value” or “PPV.” Positive predictive value can be calculated as the number of true positives divided by the sum of the true positives and false positives. PPV is determined by the characteristics of the predictive methods described herein as well as the prevalence of the condition in the population analyzed. The statistical algorithms can be selected such that the positive predictive value in a population having a condition prevalence is in the range of 70% to 99% and can be, for example, at least 70%, 75%, 76%. 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In other examples, the probability that an individual identified as not having a specified condition or outcome actually does not have that condition can be expressed as a “negative predictive value” or “NPV.” Negative predictive value can be calculated as the number of true negatives divided by the sum of the true negatives and false negatives. Negative predictive value is determined by the characteristics of the diagnostic or prognostic method, system, or code as well as the prevalence of the disease in the population analyzed. The statistical methods and models can be selected such that the negative predictive value in a population having a condition prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, a subject is determined to have a significant probability of having or not having a specified condition or outcome. By “significant probability” is meant that the subject has a reasonable probability (0.6, 0.7, 0.8, 0.9 or more) of having, or not having, a specified condition or outcome.

In some embodiments, the biomarker score is combined with one or more clinical risk scores, such as SOFA, qSOFA, or APACHE. For example, a formula is used to combine (i) either the individual gene expression values or the output from a classifier that uses the gene expression values, with (ii) the clinical risk score, to generate (iii) a new score that is useful to the clinician.

### VI. TREATMENT DECISIONS

The methods described herein may be used to classify subjects with a viral infection according to the relative risk of 30-day mortality or need for ICU care. In particular embodiments, subjects are classified as having high, low, or intermediate risk. Subjects at high risk of 30-day mortality should receive immediate intensive care. For example, patients identified as having a high risk of mortality within 30 days by the methods described herein can be sent immediately to the ICU for treatment, whereas patients identified as having a low risk of mortality within 30 days may be discharged from the emergency room setting, e.g., released from the hospital for self-isolation and further monitoring and/or treated in a regular hospital ward. Both patients and clinicians can benefit from better estimates of mortality risk, which allows timely discussions of patients' preferences and their choices regarding life-saving measures. Better molecular phenotyping of patients also makes possible improvements in clinical trials, both in 1) patient selection for drugs and interventions and 2) assessment of observed-to-expected ratios of subject mortality. A summary of the three risk classes (“low”, “intermediate” or “indeterminate”, and “high”), and exemplary treatment or triage decisions for each class, is shown in FIG. 4. As used herein. “urgent care” comprises any action taken with respect to the treatment of the subject in an emergency room or urgent care context in order to alleviate, eliminate, slow the progression of, or in any way improve any aspect or symptom of the viral infection, including, but not limited to, administering a therapeutic drug, administering organ-supportive care, and admission to an ICU.

ICU treatment of a patient, identified as having a high risk of mortality within 30 days, may comprise constant monitoring of bodily functions and providing life support equipment and/or medications to restore normal bodily function. ICU treatment may include, for example, using mechanical ventilators to assist breathing, equipment for monitoring bodily functions (e.g., heart and pulse rate, air flow to the lungs, blood pressure and blood flow, central venous pressure, amount of oxygen in the blood, and body temperature), pacemakers, defibrillators, dialysis equipment, intravenous lines, feeding tubes, suction pumps, drains, and/or catheters, and/or administering various drugs for treating the life threatening condition (e.g., sepsis, severe trauma, or bum). ICU treatment may further comprise administration of one or more analgesics to reduce pain, and/or sedatives to induce sleep or relieve anxiety, and/or barbiturates (e.g., pentobarbital or thiopental) to medically induce coma.

In certain embodiments, a critically ill patient diagnosed with a viral infection is further administered a therapeutically effective dose of an antiviral agent, such as a broad-spectrum antiviral agent, an antiviral vaccine, a neuraminidase inhibitor (e.g., zanamivir (Relenza) and oseltamivir (Tamiflu)), a nucleoside analog (e.g., acyclovir, zidovudine (AZT), and lamivudine), an antisense antiviral agent (e.g., phosphorothioate antisense antiviral agents (e.g., Fomivirsen (Vitravene) for cytomegalovirus retinitis), morpholino antisense antiviral agents), an inhibitor of viral uncoating (e.g., Amantadine and rimantadine for influenza, Pleconaril for rhinoviruses), an inhibitor of viral entry (e.g., Fuzeon for HIV), an inhibitor of viral assembly (e.g., Rifampicin), or an antiviral agent that stimulates the immune system (e.g., interferons). Exemplary antiviral agents include Abacavir, Aciclovir, Acyclovir, Adefovir, Amantadine, Amprenavir, Ampligen, Arbidol, Atazanavir, Atripla (fixed dose drug), Balavir, Cidofovir, Combivir (fixed dose drug), Dolutegravir, Darunavir. Delavirdine. Didanosine, Docosanol, Edoxudine, Efavirenz, Emtricitabine, Enfuvirtide, Entecavir, Ecoliever, Famciclovir, Fixed dose combination (antiretroviral), Fomivirsen, Fosamprenavir, Foscarnet, Fosfonet, Fusion inhibitor, Ganciclovir, Ibacitabine, Imunovir, Idoxuridine, Imiquimod. Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nitazoxanide, Nucleoside analogues, Novir, Oseltamivir (Tamiflu), Peginterferon alfa-2a, Penciclovir, Peramivir, Pleconaril. Podophyllotoxin, Protease inhibitor, Raltegravir, Reverse transcriptase inhibitor, Ribavirin, Rimantadine, Ritonavir, Pyramidine, Saquinavir, Sofosbuvir, Stavudine, Synergistic enhancer (antiretroviral), Telaprevir, Tenofovir, Tenofovir disoproxil, Tipranavir, Trifluridine, Trizivir, Tromantadine. Truvada. Valaciclovir (Valtrex), Valganciclovir, Vicriviroc. Vidarabine, Viramidine, Zalcitabine, Zanamivir (Relenza), and Zidovudine. Other drugs that may be administered include chloroqume, hydroxvchloroquine, sarilumab, remdesivir, azithronmcin, and statins.

In some embodiments, a critically ill patient diagnosed with a viral infection is further administered a therapeutically effective dose of an innate or adaptive immunity modulator such as abatacept, Abetimus, Abrilumab, adalimumab, Afelimomab, Aflibercept, Alefacept, anakinra, Andecaliximab, Anifrolumab. Anrukinzumab, Anti-lymphocyte globulin, Anti-thymocyte globulin, antifolate, Apolizumab, Apremilast. Aselizumab, Atezolizumab, Atorolimumab, Avelumab, azathioprine, Basiliximab, Belatacept, Belimumab, Benralizumab, Bertilimumab, Besilesomab, Bleselumab, Blisibimod, Brazikumab, Briakinumab, Brodalumab, Canakinumab, Carlumab, Cedelizumab. Certolizumab pegol, chloroquine. Clazakizumab, Clenoliximab, corticosteroids, cyclosporine, Daclizumab, Dupilumab, Durvalumab, Eculizumab, Efalizumab, Eldelumab, Elsilimomab, Emapalumab, Enokizumab. Epratuzumab. Erlizumab, etanercept, Etrolizumab. Everolimus, Fanolesomab, Faralimomab, Fezakinumab, Fletikumab, Fontolizumab, Fresolimumab, Galiximab. Gavilimomab, Gevokizumab, Gilvetmab, golimumab, Gomiliximab, Guselkumab, Gusperimus, hydroxychloroquine. Ibalizumab, Immunoglobulin E, Inebilizumab, infliximab, Inolimomab, Integrin, Interferon, Ipilimumab, Itolizumab, Ixekizumab, Keliximab, Lampalizumab, Lanadelumab, Lebrikizumab, leflunomide, Lemalesomab, Lenalidomide, Lenzilumab, Lerdelimumab, Letolizumab, Ligelizumab, Lirilumab, Lulizumab pegol, Lumiliximab, Maslimomab. Mavrilimumab, Mepolizumab, Metelimumab, methotrexate, minocycline, Mogamulizumab. Morolimumab, Muromonab-CD3. Mycophenolic acid. Namilumab, Natalizumab, Nerelimomab, Nivolumab, Obinutuzumab, Ocrelizumab, Odulimomab, Oleclumab, Olokizumab, Omalizumab. Otelixizumab, Oxelumab, Ozoralizumab, Pamrevlumab. Pascolizumab, Pateclizumab, PDE4 inhibitor. Pegsunercept, Pembrolizumab, Perakizumab, Pexelizumab, Pidilizumab, Pimecrolimus, Placulumab, Plozalizumab, Pomalidomide, Priliximab, purine synthesis inhibitors, pyrimidine synthesis inhibitors, Quilizumab, Reslizumab. Ridaforolimus, Rilonacept, rituximab, Rontalizumab, Rovelizumab, Ruplizumab, Samalizumab, Sarilumab, Secukinumab, Sifalimumab. Siplizumab, Sirolimus, Sirukumab, Sulesomab, sulfasalazine, Tabalumab, Tacrolimus, Talizumab, Telimomab aritox, Temsirolimus, Teneliximab, Teplizumab, Teriflunomide, Tezepelumab, Tildrakizumab, tocilizumab, tofacitinib, Toralizumab, Tralokinumab, Tregalizumab, Tremelimumab. Ulocuplumab, Umirolimus, Urelumab, Ustekinumab, Vapaliximab, Varlilumab, Vatelizumab, Vedolizumab, Vepalimomab, Visilizumab, Vobarilizumab, Zanolimumab, Zolimomab aritox, Zotarolimus, or recombinant human cytokines, such as rh-interferon-gamma.

In some embodiments, a critically ill patient diagnosed with a viral infection is further administered a therapeutically effective dose of a blockade or signaling modification of PD1, PDL1, CTLA4, TIM-3, BTLA, TREM-1, LAG3, VISTA, or any of the human clusters of differentiation, including CD1, CD1a, CD1b, CD1c, CD1d, CD1e, CD2, CD3, CD3d. CD3e, CD3g, CD4, CD5, CD6, CD7, CD8. CD8a, CD8b, CD9, CD10, CD11a, CD11b. CD11c, CD11d, CD13, CD14, CD15, CD16, CD16a. CD16b, CD17, CD18, CD19, CD20, CD21. CD22, CD23, CD24. CD25, CD26, CD27. CD28, CD29, CD30, CD31, CD32A, CD32B. CD33, CD34, CD35, CD36, CD37, CD38, CD39, CD40, CD41, CD42, CD42a, CD42b, CD42c, CD42d, CD43, CD44, CD45, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD60b, CD60c, CD61, CD62E, CD62L, CD62P, CD63, CD64a, CD65, CD65s, CD66a, CD66b, CD66c. CD66d. CD66e, CD66f, CD68, CD69, CD70, CD71. CD72, CD73, CD74, CD75, CD75s, CD77, CD79A, CD79B, CD80, CD81, CD82, CD83, CD84, CD85A, CD85B, CD85C, CD85D, CD85F, CD85G, CD85H, CD851, CD85J, CD85K, CD85M, CD86. CD87, CD88, CD89, CD90, CD91, CD92. CD93, CD94, CD95, CD96, CD97, CD98, CD99, CD100, CD101, CD102, CD103, CD104, CD105, CD106, CD107, CD107a, CD107b, CD108, CD109, CD110, CD111, CD112, CD113, CD114, CD115, CD116, CD117, CD118, CD119, CD120, CD120a, CD120b, CD121a, CD121b, CD122, CD123, CD124, CD125, CD126, CD127, CD129, CD130, CD131, CD132, CD133. CD134, CD135, CD136, CD137, CD138, CD139, CD140A, CD140B, CD141, CD142, CD143, CD144, CDw145, CD146, CD147, CD148, CD150, CD151, CD152, CD153, CD154, CD155, CD156, CD156a, CD156b, CD156c, CD157, CD158, CD158A, CD158B1, CD158B2, CD158C, CD158D, CD158E1, CD158E2, CD158F1, CD158F2, CD158G, CD158H, CD158I, CD158J, CD158K, CD159a, CD159c, CD160, CD161, CD162, CD163, CD164, CD165, CD166, CD167a, CD167b, CD168, CD169, CD170, CD171, CD172a, CD172b, CD172g, CD173, CD174, CD175, CD175s, CD176, CD177, CD178, CD179a. CD179b, CD180, CD181, CD182, CD183, CD184, CD185, CD186, CD187, CD188, CD189, CD190, CD191, CD192, CD193, CD194, CD195, CD196, CD197, CDw198, CDw199, CD200, CD201, CD202b, CD203c, CD204, CD205, CD206, CD207, CD208, CD209, CD210, CDw210a. CDw210b, CD211, CD212, CD213al, CD213a2, CD214, CD215, CD216, CD217, CD218a, CD218b, CD219, CD220, CD221, CD222, CD223, CD224, CD225, CD226, CD227, CD228, CD229, CD230, CD231, CD232, CD233, CD234, CD235a, CD235b, CD236, CD237. CD238. CD239, CD240CE, CD240D, CD241, CD242, CD243. CD244, CD245, CD246, CD247, CD248, CD249, CD250, CD251, CD252, CD253, CD254, CD255, CD256, CD257, CD258, CD259, CD260, CD261, CD262, CD263, CD264, CD265, CD266, CD267, CD268, CD269, CD270, CD271, CD272, CD273, CD274, CD275, CD276, CD277, CD278, CD279, CD280, CD281, CD282, CD283, CD284, CD285, CD286, CD287, CD288, CD289, CD290, CD291, CD292, CDw293, CD294, CD295, CD296, CD297, CD298, CD299, CD300A, CD300C, CD301, CD302, CD303, CD304, CD305, CD306, CD307, CD307a, CD307b, CD307c, CD307d, CD307e. CD308. CD309. CD310. CD311. CD312, CD313, CD314, CD315, CD316, CD317, CD318, CD319, CD320, CD321, CD322, CD323, CD324, CD325, CD326, CD327, CD328, CD329, CD330, CD331, CD332, CD333, CD334, CD335, CD336, CD337, CD338, CD339, CD340, CD344, CD349, CD351, CD352, CD353, CD354, CD355, CD357, CD358, CD360, CD361, CD362, CD363, CD364, CD365, CD366, CD367, CD368, CD369, CD370, or CD371.

In some embodiments, a critically ill patient diagnosed with a viral infection is further administered a therapeutically effective dose of one or more drugs that modify the coagulation cascade or platelet activation, such as those targeting Albumin, Antihemophilic globulin, AHF A, C1-inhibitor, Ca++, CD63, Christmas factor, AHF B, Endothelial cell growth factor, Epidermal growth factor, Factors V, XI, XIII, Fibrin-stabilizing factor, Laki-Lorand factor, fibrinase, Fibrinogen, Fibronectin, GMP 33, Hageman factor, High-molecular-weight kininogen, IgA, IgG, IgM, Interleukin-IB, Multimerin, P-selectin, Plasma thromboplastin antecedent, AHF C, Plasminogen activator inhibitor 1, Platelet factor. Platelet-derived growth factor, Prekallikrein, Proaccelerin, Proconvertin, Protein C. Protein M, Protein S. Prothrombin, Stuart-Prower factor, TF, thromboplastin, Thrombospondin, Tissue factor pathway inhibitor, Transforming growth factor-β. Vascular endothelial growth factor, Vitronectin, von Willebrand factor, α2-Antiplasmin, α2-Macroglobulin. β-Thromboglobulin, or other members of the coagulation or platelet-activation cascades.

### VII. KITS AND SYSTEMS

A. Kits

In one aspect, kits are provided for prognosis of mortality in a subject, wherein the kits can be used to detect the biomarkers described herein. For example, the kits can be used to detect any one or more of the biomarkers described herein, which are differentially expressed in samples from 30-day survivors and non-survivors in subjects with viral infections. The kit may include one or more agents for detection of biomarkers, a container for holding a biological sample isolated from a human subject suspected of having a viral infection; and printed instructions for reacting agents with the biological sample or a portion of the biological sample to detect the presence or amount of at least one biomarker in the biological sample. The agents may be packaged in separate containers. The kit may further comprise one or more control reference samples and reagents for performing a PCR, isothermal amplification, immunoassay. NanoString, or microarray analysis, e.g., reference samples from subjects with a survivor or non-survivor outcome at 30 days. The kit may also comprise one or more devices or implements for carrying out any of the herein devices. e.g., 96-well plates, microfluidic cartridges, single-well multiplex assays, etc.

In certain embodiments, the kit comprises agents for measuring the levels of at least five or six biomarkers of interest. For example, the kit may include agents, e.g., primers and/or probes, for detecting biomarkers of a panel comprising a TGFBI polynucleotide, a DEFA4 polynucleotide, a LY86 polynucleotide, a BATF polynucleotide, and an HK3 polynucleotide. In some embodiments, the panel further comprises HLA-DPB1. In some embodiments, the panel comprises any one or more of the biomarkers listed in Table 1 or Table 5. In some embodiments, the panel comprises any one or more pairs of biomarkers listed in Table 3 or Table 6.

In certain embodiments, the kit comprises a microarray or other solid support for analysis of a plurality of biomarker polynucleotides. An exemplary microarray or other support included in the kit comprises an oligonucleotide that hybridizes to a TGFBI polynucleotide, an oligonucleotide that hybridizes to a DEFA4 polynucleotide, an oligonucleotide that hybridizes to a LY86 polynucleotide, an oligonucleotide that hybridizes to a BATF polynucleotide, and an oligonucleotide that hybridizes to an HK3 polynucleotide. In some embodiments, the kit further comprises an oligonucleotide that hybridizes to an HLA-DPB1 polynucleotide. In some embodiments, the microarray or other support comprises an oligonucleotide for each of the biomarkers detected using the herein-described methods, including biomarkers listed in Tables 1 and 5 or pairs of biomarkers listed in Tables 3 and 6.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of diagnosing or evaluating a viral infection.

B. Measurement Systems for Detecting and Recording Biomarker Expression

In one aspect, a measurement system is provided. Such systems allow, e.g., the detection of biomarker gene expression in a sample and the recording of the data resulting from the detection. The stored data can then be analyzed as described elsewhere herein to determine the virus infection status of a subject. Such systems can comprise assay systems (e.g., comprising an assay device and detector), which can transmit data to a logic system (such as a computer or other system or device for capturing, transforming, analyzing, or otherwise processing data from the detector). The logic system can have any one or more of multiple functions, including controlling elements of the overall system such as the assay system, sending data or other information to a storage device or external memory, and/or issuing commands to a treatment device.

An exemplary measurement system is shown in FIG. 16. The system as shown includes a sample 1605, such as cell-free DNA molecules within an assay device 1610, where an assay 1608 can be performed on sample 705. For example, sample 1605 can be contacted with reagents of assay 1608 to provide a signal of a physical characteristic 1615. An example of an assay device can be a flow cell that includes probes and/or primers of an assay or a tube through which a droplet moves (with the droplet including the assay). Physical characteristic 1615 (e.g., a fluorescence intensity, a voltage, or a current), from the sample is detected by detector 1620. Detector 1620 can take a measurement at intervals (e.g., periodic intervals) to obtain data points that make up a data signal. In one embodiment, an analog-to-digital converter converts an analog signal from the detector into digital form at a plurality of times. Assay device 1610 and detector 1620 can form an assay system, e.g., an amplification and detection system that measures biomarker gene expression according to embodiments described herein. A data signal 1625 is sent from detector 1620 to logic system 1630. As an example, data signal 1625 can be used to determine expression levels for selected biomarkers. Data signal 1625 can include various measurements made at a same time, e.g., different colors of fluorescent dyes or different electrical signals for different molecules of sample 1605, and thus data signal 1625 can correspond to multiple signals. Data signal 1625 may be stored in a local memory 1635, an external memory 1640, or a storage device 1645. System 1600 may also include a treatment device 1660, which can provide a treatment to the subject. Treatment device 1660 can determine a treatment and/or be used to perform a treatment. Examples of such treatment can include surgery, radiation therapy, chemotherapy, immunotherapy, targeted therapy, hormone therapy, and stem cell transplant. Logic system 1630 may be connected to treatment device 1660, e.g., to provide results of a method described herein. The treatment device may receive inputs from other devices, such as an imaging device and user inputs (e.g., to control the treatment, such as controls over a robotic system).

Certain aspects of the herein-described methods may be totally or partially performed with a computer system including one or more processors, which can be configured to perform the steps. Thus, embodiments are directed to computer systems configured to perform the steps of methods described herein, potentially with different components performing a respective step or a respective group of steps. The computer systems of the present disclosure can be part of a measuring system as described above, or can be independent of any measuring systems. In some embodiments, the present disclosure provides a computer system that calculates a viral score based on inputted biomarker expression (and optionally other) data, and determines the 30-day mortality risk of a subject.

An exemplary computer system is shown in FIG. 17. Any of the computer systems may utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices. The subsystems shown in FIG. 17 are interconnected via a system bus 175. Additional subsystems such as a printer 174, keyboard 178, storage device(s) 179, monitor 176 (e.g., a display screen, such as an LED), which is coupled to display adapter 182, and others are shown. Peripherals and input/output (I/O) devices, which couple to I/O controller 171, can be connected to the computer system by any number of means known in the art such as input/output (I/O) port 177 (e.g., USB, FireWire*). For example, I/O port 177 or external interface 181 (e.g. Ethernet, Wi-Fi, etc.) can be used to connect computer system 180 to a wide area network such as the Internet, a mouse input device, or a scanner. The interconnection via system bus 175 allows the central processor 173 to communicate with each subsystem and to control the execution of a plurality of instructions from system memory 172 or the storage device(s) 179 (e.g., a fixed disk, such as a hard drive, or optical disk), as well as the exchange of information between subsystems. The system memory 172 and/or the storage device(s) 179 may embody a computer readable medium. Another subsystem is a data collection device 185, such as a camera, microphone, accelerometer, and the like. Any of the data mentioned herein can be output from one component to another component and can be output to the user. A computer system can include a plurality of the same components or subsystems. e.g., connected together by external interface 181, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

In one aspect, the disclosure provides a computer implemented method for determining 30-day mortality risk of a patient having a viral infection. The computer performs steps comprising, e.g., receiving inputted patient data comprising values for the levels of one or more biomarkers in a biological sample from the patient; analyzing the levels of one or more biomarkers and optionally comparing them to respective reference values, e.g., to a housekeeping reference gene for normalization: calculating a 30-day mortality score for the patient based on the levels of the biomarkers and comparing the score to one or more threshold values to assign the patient to a risk category; and displaying information regarding the mortality risk of the patient. In certain embodiments, the inputted patient data comprises values for the levels of a plurality of biomarkers in a biological sample from the patient. In one embodiment, the inputted patient data comprises values for the levels of TGFBI, DEFA4, LY86, BATF and HK3 polynucleotides. In one embodiment, the inputted patient data comprises values for the levels of TGFBI, DEFA4, LY86, BATF, HK3, and HLA-DPB1.

In a further aspect, a diagnostic system is provided for performing the computer implemented method, as described. A diagnostic system may include a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions for determining the mortality risk of the subject. For example, the storage component includes instructions for calculating the mortality gene score for the subject based on biomarker expression levels, as described herein. In addition, the storage component may further comprise instructions for performing multivariate linear discriminant analysis (LDA), receiver operating characteristic (ROC) analysis, principal component analysis (PCA), ensemble data mining methods, cell specific significance analysis of microarrays (csSAM), or multi-dimensional protein identification technology (MUDPIT) analysis. The computer processor is coupled to the storage component and configured to execute the instructions stored in the storage component in order to receive patient data and analyze patient data according to one or more algorithms. The display component displays information regarding the diagnosis and/or prognosis (e.g., mortality risk) of the patient. The storage component may be of any type capable of storing information accessible by the processor, such as a hard-drive, memory card, ROM, RAM, DVD, CD-ROM, USB Flash drive, write-capable, and read-only memories.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the diagnostic system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data. In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may actually comprise a collection of processors which may or may not operate in parallel. In one aspect, computer is a server communicating with one or more client computers. Each client computer may be configured similarly to the server, with a processor, storage component and instructions. Although the client computers and may comprise a full-sized personal computer, many aspects of the system and method are particularly advantageous when used in connection with mobile devices capable of wirelessly exchanging data with a server over a network such as the Internet.

### VIII. EXAMPLES

The following examples are offered to illustrate, but not to limit, the claimed disclosure.

### A. Example 1. Genome-Wide Analysis of 27 Cohort Data

To assess the feasibility of signature gene identification for viral severity in host response, we looked at genome-wide gene expression data of 856 viral infected patients. 15 top genes were selected, and their 2-gene pairs were evaluated for differentiating non-survival cases from survival cases.

1. Data Sets

We used a collection of blood gene expression data of 5,217 patients from 42 studies including bacterial and viral infections and healthy controls (IMX11). This genome-wide mRNA profile included 13.902 genes and was co-normalized using the well-tested COCONUT method across multiple platforms. We selected all viral cases of 856 patients from 27 cohorts. Of these 856 patients, 691 are annotated as survival within 28 or 30 days, 4 as non-survival within 28 or 30 days, and 161 as unknown. This viral severity analysis was performed for two group comparison between 4 non-survival cases (positive) and 691 survival cases (negative).

2. Methods

Several metrics for contrasting two groups were applied to non-survival vs. survival cases to select genes of interest, including Pearson correlation, Kendall rank correlation, Spearman rank correlation, t-test, and other non-parametric measures. Given the extremely imbalanced cases between two groups (4 vs. 691), neither over-sampling of the non-survival group nor under-sampling of the survival group can be reliably applied. The significance we estimated for each test, either analytically with a multiplicity correction or by permutations, were mainly used for the purpose of ranking genes and suggesting cutoff values given the statistical power severely limited by the small number of non-survival cases.

3. Results

We examined the results of top genes from each metric guided by the rough significance estimate. We found that top genes from different metrics are highly overlapped, showing a degree of concordant results amongst various metrics used. Hence, we heuristically decided to select top 10 genes from only two methods: Pearson correlation representing numeric-based test category, and Kendall correlation, representing rank-based test category, resulting in a total of 15 genes.

To check the performance of these 15 genes in terms of predicting the viral severity, we used gene expression measurements from each of these 15 genes in all patients as predictor and calculated the AUROC values shown in Table 1 (0.898-0.994).

TABLE 1
AUROC for each of 15 selected genes.

| Gene | AUROC |
|---|---|
| TDRD1 | 0.920 |
| POLE | 0.990 |
| MYOM1 | 0.957 |
| PDZD4 | 0.899 |
| HHLA3 | 0.976 |
| PDE4B | 0.983 |
| HSPA14 | 0.990 |
| PRDM2 | 0.980 |
| TSPAN13 | 0.982 |
| GAB4 | 0.985 |
| RPL4 | 0.994 |
| EGLN1 | 0.991 |
| TRIM67 | 0.985 |
| AACS | 0.984 |
| ST8SIA3 | 0.981 |

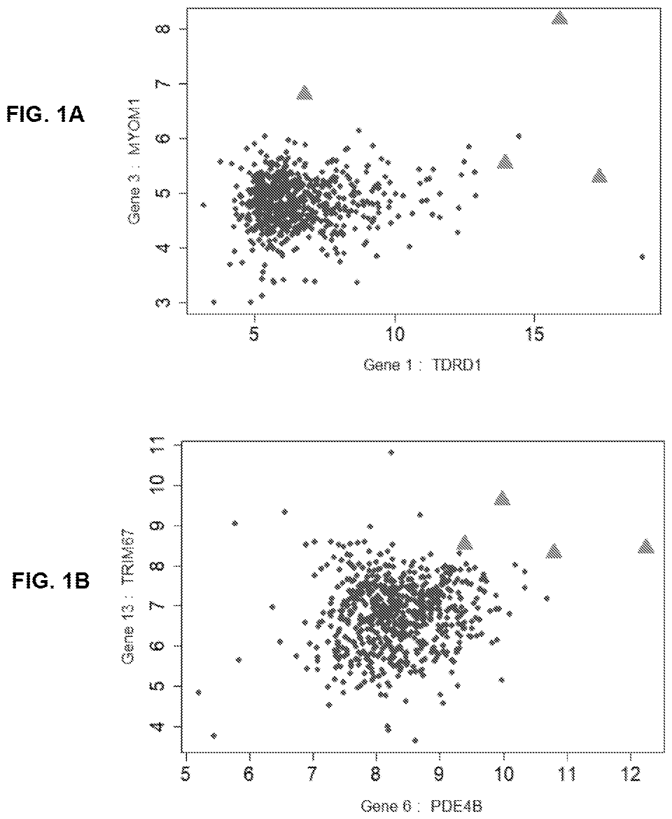

We then assessed each of 2-gene combinations out of these 15 genes by using their geometric mean of each pair as a prediction score and calculated their AUROCs (0.940-0.998). Two examples of such 105 gene pairs are illustrated in FIG. 1. The distribution of all AUROCs from all 105 pairs is shown in FIG. 2B. The AUROCs for each of the two-gene pairs is shown in Table 3.

We also calculated AUROCs using geometric mean as a prediction score for a series of models starting with one gene and recursively adding one up to 15 genes based on the ranked order in Table 1. The results are reported in Table 2 (0.920-0.997).

TABLE 2
AUROC for a model sequentially using 1, 2, and up to 15 genes.

| # Genes | AUROC |
|---|---|
| 1 | 0.920 |
| 2 | 0.993 |
| 3 | 0.997 |
| 4 | 0.996 |
| 5 | 0.995 |
| 6 | 0.996 |
| 7 | 0.997 |
| 8 | 0.996 |
| 9 | 0.996 |
| 10 | 0.996 |
| 11 | 0.996 |
| 12 | 0.996 |
| 13 | 0.997 |
| 14 | 0.996 |
| 15 | 0.996 |

TABLE 3

| 2-gene pair | AUROC |
|---|---|
| 2-gene pair 1: TDRD1 - POLE | 0.993 |
| 2-gene pair 2: TDRD1 - MYOM1 | 0.984 |
| 2-gene pair 3: TDRD1 - PDZD4 | 0.973 |
| 2-gene pair 4: TDRD1 - HHLA3 | 0.978 |
| 2-gene pair 5: TDRD1 - PDE4B | 0.968 |
| 2-gene pair 6: TDRD1 - HSPA14 | 0.979 |
| 2-gene pair 7: TDRD1 - PRDM2 | 0.987 |
| 2-gene pair 8: TDRD1 - TSPAN13 | 0.986 |
| 2-gene pair 9: TDRD1 - GAB4 | 0.977 |
| 2-gene pair 10: TDRD1 - RPL4 | 0.989 |
| 2-gene pair 11: TDRD1 - EGLN1 | 0.984 |
| 2-gene pair 12: TDRD1 - TRIM67 | 0.982 |
| 2-gene pair 13: TDRD1 -- AACS | 0.975 |
| 2-gene pair 14: TDRD1 - ST8SIA3 | 0.969 |
| 2-gene pair 15: POLE - MYOM1 | 0.993 |
| 2-gene pair 16: POLE - PDZD4 | 0.979 |
| 2-gene pair 17: POLE - HHLA3 | 0.988 |
| 2-gene pair 18: POLE - PDE4B | 0.995 |
| 2-gene pair 19: POLE - HSPA14 | 0.996 |
| 2-gene pair 20: POLE - PRDM2 | 0.986 |
| 2-gene pair 21: POLE - TSPAN13 | 0.990 |
| 2-gene pair 22: POLE - GAB4 | 0.994 |
| 2-gene pair 23: POLE - RPL4 | 0.994 |
| 2-gene pair 24: POLE - EGLN1 | 0.992 |
| 2-gene pair 25: POLE - TRIM67 | 0.994 |
| 2-gene pair 26: POLE -- AACS | 0.990 |
| 2-gene pair 27: POLE - ST8SIA3 | 0.990 |
| 2-gene pair 28: MYOM1 - PDZD4 | 0.940 |
| 2-gene pair 29: MYOM1 - HHLA3 | 0.987 |
| 2-gene pair 30: MYOM1 - PDE4B | 0.982 |
| 2-gene pair 31: MYOM1 - HSPA14 | 0.997 |
| 2-gene pair 32: MYOM1 - PRDM2 | 0.985 |
| 2-gene pair 33: MYOM1 - TSPAN13 | 0.993 |
| 2-gene pair 34: MYOM1 - GAB4 | 0.987 |
| 2-gene pair 35: MYOM1 - RPL4 | 0.995 |
| 2-gene pair 36: MYOM1 - EGLN1 | 0.993 |
| 2-gene pair 37: MYOM1 - TRIM67 | 0.996 |
| 2-gene pair 38: MYOM1 -- AACS | 0.991 |
| 2-gene pair 39: MYOM1 - ST8SIA3 | 0.989 |
| 2-gene pair 40: PDZD4 - HHLA3 | 0.961 |
| 2-gene pair 41: PDZD4 - PDE4B | 0.945 |
| 2-gene pair 42: PDZD4 - HSPA14 | 0.974 |
| 2-gene pair 43: PDZD4 - PRDM2 | 0.962 |
| 2-gene pair 44: PDZD4 - TSPAN13 | 0.975 |
| 2-gene pair 45: PDZD4 - GAB4 | 0.952 |
| 2-gene pair 46: PDZD4 - RPL4 | 0.983 |
| 2-gene pair 47: PDZD4 - EGLN1 | 0.970 |
| 2-gene pair 48: PDZD4 - TRIM67 | 0.965 |
| 2-gene pair 49: PDZD4 -- AACS | 0.977 |
| 2-gene pair 50: PDZD4 - ST8SIA3 | 0.951 |
| 2-gene pair 51: HHLA3 - PDE4B | 0.990 |
| 2-gene pair 52: HHLA3 - HSPA14 | 0.996 |
| 2-gene pair 53: HHLA3 - PRDM2 | 0.981 |
| 2-gene pair 54: HHLA3 - TSPAN13 | 0.987 |
| 2-gene pair 55: HHLA3 - GAB4 | 0.990 |
| 2-gene pair 56: HHLA3 - RPL4 | 0.993 |
| 2-gene pair 57: HHLA3 - EGLN1 | 0.991 |
| 2-gene pair 58: HHLA3 - TRIM67 | 0.993 |
| 2-gene pair 59: HHLA3 -- AACS | 0.986 |
| 2-gene pair 60: HHLA3 - ST8SIA3 | 0.986 |
| 2-gene pair 61: PDE4B - HSPA14 | 0.997 |
| 2-gene pair 62: PDE4B - PRDM2 | 0.988 |
| 2-gene pair 63: PDE4B - TSPAN13 | 0.991 |
| 2-gene pair 64: PDE4B - GAB4 | 0.991 |
| 2-gene pair 65: PDE4B - RPL4 | 0.996 |
| 2-gene pair 66: PDE4B - EGLN1 | 0.994 |
| 2-gene pair 67: PDE4B - TRIM67 | 0.999 |
| 2-gene pair 68: PDE4B -- AACS | 0.990 |
| 2-gene pair 69: PDE4B - ST8SIA3 | 0.991 |
| 2-gene pair 70: HSPA14 - PRDM2 | 0.992 |
| 2-gene pair 71: HSPA14 - TSPAN13 | 0.992 |
| 2-gene pair 72: HSPA14 - GAB4 | 0.994 |
| 2-gene pair 73: HSPA14 - RPL4 | 0.996 |
| 2-gene pair 74: HSPA14 - EGLN1 | 0.997 |
| 2-gene pair 75: HSPA14 - TRIM67 | 0.997 |
| 2-gene pair 76: HSPA14 -- AACS | 0.993 |
| 2-gene pair 77: HSPA14 - ST8SIA3 | 0.994 |
| 2-gene pair 78: PRDM2 - TSPAN13 | 0.986 |
| 2-gene pair 79: PRDM2 - GAB4 | 0.987 |
| 2-gene pair 80: PRDM2 - RPL4 | 0.992 |
| 2-gene pair 81: PRDM2 - EGLN1 | 0.987 |
| 2-gene pair 82: PRDM2 - TRIM67 | 0.990 |
| 2-gene pair 83: PRDM2 -- AACS | 0.984 |
| 2-gene pair 84: PRDM2 - ST8SIA3 | 0.983 |
| 2-gene pair 85: TSPAN13 - GAB4 | 0.989 |
| 2-gene pair 86: TSPAN13 - RPL4 | 0.992 |
| 2-gene pair 87: TSPAN13 - EGLN1 | 0.989 |
| 2-gene pair 88: TSPAN13 - TRIM67 | 0.988 |
| 2-gene pair 89: TSPAN13 -- AACS | 0.985 |
| 2-gene pair 90: TSPAN13 - ST8SIA3 | 0.984 |
| 2-gene pair 91: GAB4 - RPL4 | 0.994 |
| 2-gene pair 92: GAB4 - EGLN1 | 0.995 |
| 2-gene pair 93: GAB4 - TRIM67 | 0.993 |
| 2-gene pair 94: GAB4 - AACS | 0.989 |
| 2-gene pair 95: GAB4 - ST8SIA3 | 0.991 |
| 2-gene pair 96: RPL4 - EGLN1 | 0.993 |
| 2-gene pair 97: RPL4 - TRIM67 | 0.994 |
| 2-gene pair 98: RPL4 -- AACS | 0.993 |
| 2-gene pair 99: RPL4 - ST8SIA3 | 0.993 |
| 2-gene pair 100: EGLN1 - TRIM67 | 0.996 |
| 2-gene pair 101: EGLN1 -- AACS | 0.990 |
| 2-gene pair 102: EGLN1 - ST8SIA3 | 0.989 |
| 2-gene pair 103: TRIM67 -- AACS | 0.991 |
| 2-gene pair 104: TRIM67 - ST8SIA3 | 0.991 |
| 2-gene pair 105: AACS - ST8SIA3 | 0.984 |

To summarize, FIGS. 2A-2D display histograms of AUROCs for the three scenarios above (FIGS. 2A-2C) in comparison with a distribution where each of 13,902 genes in the data is used to calculate AUROC (FIG. 2D). The difference in AUROC distributions between the three scenarios involving the 15 selected genes and the full complement of 13,902 examined genes highlights the efficacy of methods using the 15 genes to predict viral severity, including when they are used in combination.

4. Discussion

The available gene expression data allowed us to identify top genes related to viral severity. Limited by the small number of mortality cases, it was not possible to use rigorous strategies such as using cross-validation and dividing data sets to training and validation set.

### B. Example 2. Identification of Viral Mortality Markers from Among 29 Genes Associated with Acute Infections

1. Data

We have previously compiled a multi-platform database of normalized gene expression data with adjudicated infection status and mortality information, from public sources and internal studies. The data contained gene expression of 29 genes found to be associated with acute infections in previous research (Mayhew et al., 2020 Nature Commun. 11, Art. 1177).

To develop a viral mortality predictor, we focused on adult patients diagnosed with viral infections and known (28 or 30)-day mortality status, where 28 or 30 were used interchangeably and are herein referred to as 30-day mortality. However, in the available data, the number of cases rate was too low for robust model development. To mitigate the situation, we applied an advanced variant of previously validated, high-performing bacterial/viral/noninfected classifier (Mayhew et al., 2020), and retained all samples with a probability of viral infection exceeding 0.5 in the three-class classifier. This increased the size of the viral dataset, and resulted in the training set of 705 29-dimensional samples, with mortality rate of 3.3% (23 samples). This data was used as input to the machine learning workflow.

2. Analysis

We applied an in-house machine learning workflow to the viral mortality training data. Due to data size, it was not possible to set aside a separate validation set; instead, the workflow used cross-validation. We found that the leave-one-study-out approach, whereas cross-validation folds comprise samples from a single study, produced the most robust results. We applied hyperparameter tuning over a search space of parameters previously found to be effective for model optimization in the infectious disease diagnosis domain. The search space size was fixed to 100, for rapid turnaround, and to limit overfitting. We only investigated linear classifiers, to limit overfitting: Support Vector Machine with linear kernel; logistic regression; and multi-layer perceptron with linear activation function.

To facilitate transfer to PCR platform, we applied feature (gene) selection, targeting 5 genes. The feature selection used univariate ranking with absolute value of Pearson correlation between gene expression and outcome as the ranking metric. The ranking was performed within the cross-validation loop to minimize bias. The final list of 5 genes was based on the average gene ranking among the cross-validation folds.

In the absence of a validation set, there is no practically viable way to produce a Receiver Operator Characteristic plot of the winning classifier on independent data. Instead, we generated two related plots based on cross-validation: 1) sensitivity and false positive rate for each model and decision threshold evaluated during the hyperparameter search; and 2) ROC-like plot based on pooled cross-validated probabilities for the best model.

Since age is a significant predictor of 30-day mortality, to assess whether our predictor of mortality is independent of age, we fit a multivariate generalized linear binomial model with our predictor and age as independent variables, and outcome as dependent variable.

3. Results

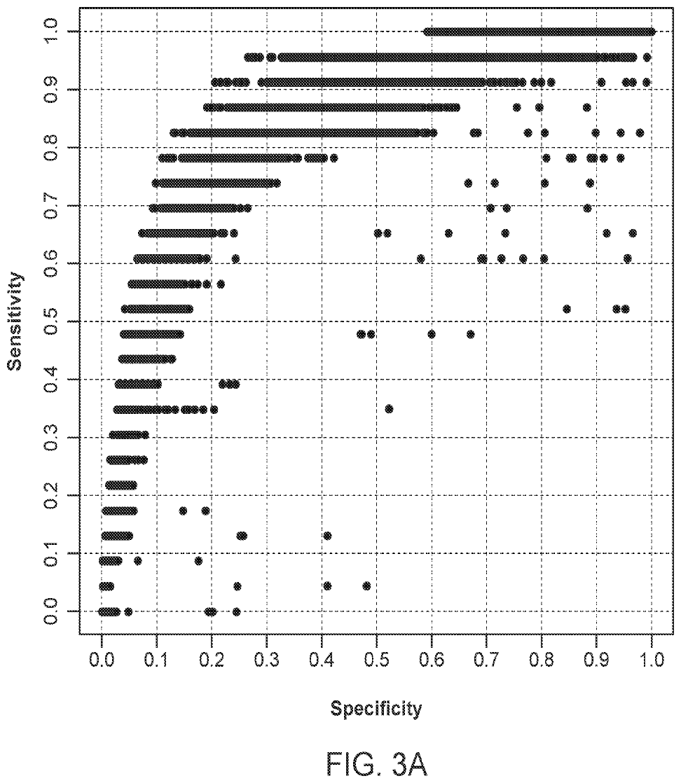

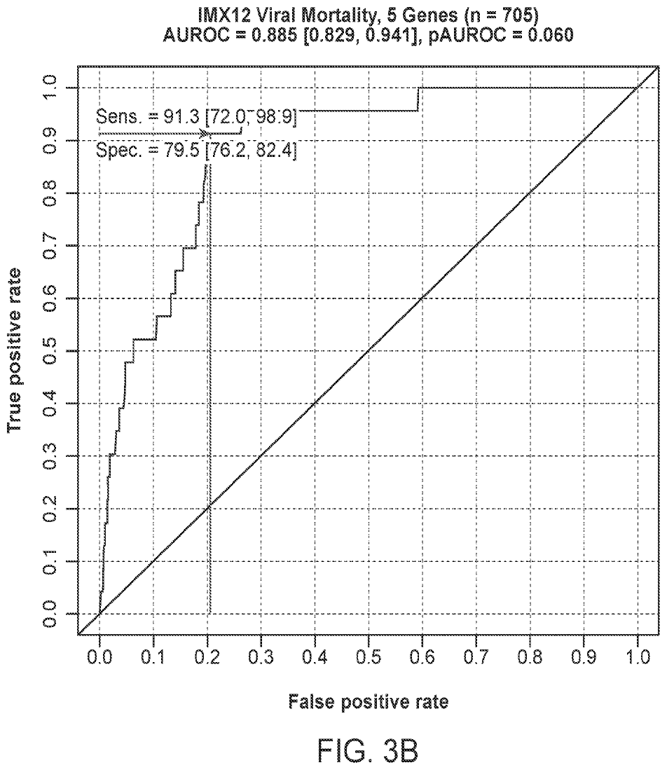

The best model (AUROC 0.89) used logistic regression and the following genes: TGFBI, DEFA4, LY86, BATF and HK3. The model selection dotplot is shown in FIG. 3A. We chose the hyperparameter configuration with the maximum AUC. The corresponding ROC is shown in FIG. 3B. Since age is a significant predictor of 30-day mortality, to assess whether our predictor of mortality is independent of age, we fit a multivariate generalized linear binomial model with our predictor and age as independent variables: the 5-gene score was significant (p<1e-6), but age was not (p=0.4).

To further characterize performance of the chosen model, we partitioned the estimated probability of death at 30 days in 3 bins: low likelihood (bin 1), intermediate (or indeterminate) (bin 2), and high-likelihood (bin 3). The bins are defined such that the likelihood ratios are <0.15 in bin 1 and >5 in bin 3. The lowest bin has an LR-0.1, sensitivity 91% (estimated NPV 99.7%); the highest bin has an LR+5, specificity 89%. The top and bottom bin thus have a DOR of ˜50, compared to procalcitonin OR 5 for COVID-19. HostDx-ViralSeverity could thus be used both to rule out hospitalization in roughly 77% of patients in the lowest-risk group, while identifying the 13% of patients at greatest need of hospitalization (FIG. 4). The cross-validation performance of the winning model, based on the split, are shown in Table 4.

Table 4 shows cross-validation performance estimates of the best model. LR=likelihood ratio. Fraction: percentage of samples assigned to the corresponding bin. Low risk bin specificity: percentage of positive samples assigned to low risk bin. High risk bin sensitivity: percentage of negative samples assigned to high risk bin. Sens@Spec90: sensitivity of best model with specificity >90%. Spec@Sens90: specificity of best model with sensitivity >90%.

TABLE 4

| Metric | Estimate |
|---|---|
| AUC | 0.885 |
| Low risk bin LR | 0.11 |
| Low risk bin fraction | 77.2% |
| Low risk bin sensitivity | 91.3% |
| High risk bin LR | 5.01 |
| High risk bin fraction | 12.8% |
| High risk bin specificity | 88.7% |
| Sens@Spec90 | 70% |
| Spec@Sens90 | 79% |

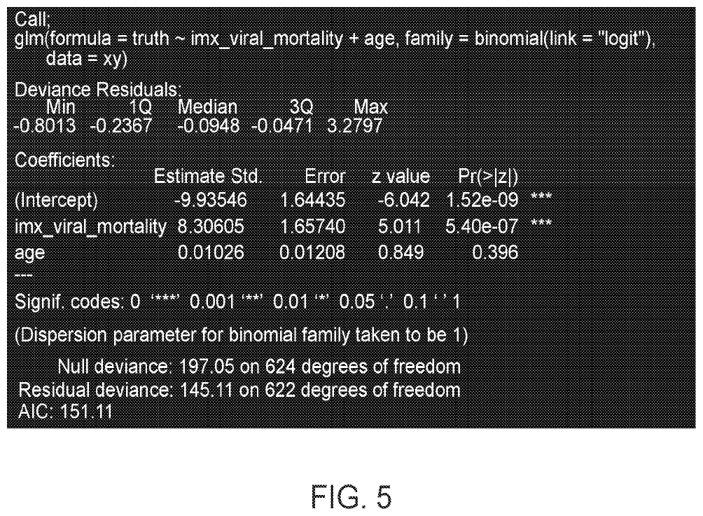

FIG. 5 contains results of adjusting the viral mortality predictor for age. The results show that the predictor contains strong prognostic information independent of age.

### C Example 3. Validation of the 5-mRNA Score

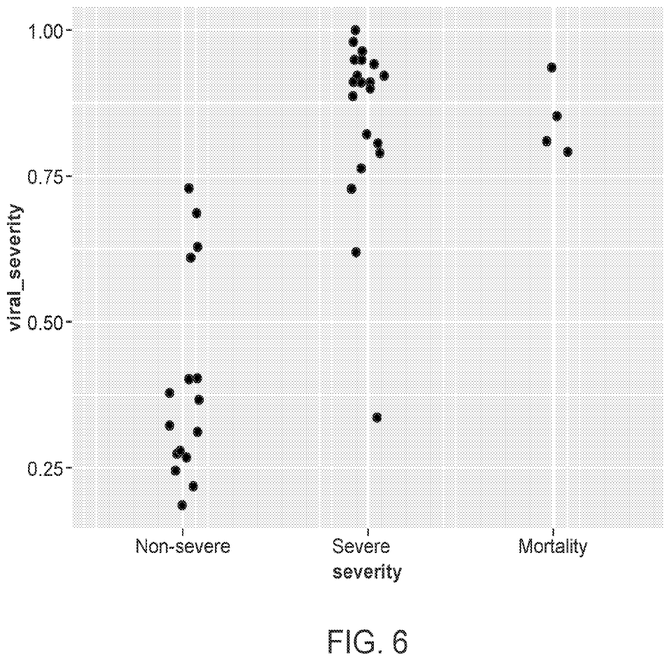

A prospective validation of the 5-mRNA score was accomplished at a single hospital in Athens. Greece. Patients were enrolled if they were SARS-COV-2 positive by PCR in the emergency department, or were transferred into the hospital with a SARS-COV-2 diagnosis and intubated. Clinical data were recorded at 30 days, including need for ICU care and/or mechanical ventilation; mortality; and other standard outcomes. Blood was taken at enrollment in PAXgene RNA tubes and shipped frozen to Inflammatix. RNA was extracted and run on the NanoString nCounter device using a custom codeset. The 5-gene score was calculated after normalization and compared to 30-day outcomes (FIG. 6).

### D. Example 4. Identification of Biomarkers Associated with Severe Response to SARS-CoV-2 Infection in Whole Blood of COVID-19 Patients for Risk Stratification

1. Summary

In response to the pandemic caused by SARS-CoV-2, we used genome-wide gene expression to study host response in blood from 62 COVID-19 patients that comprised of 39 non-severe and 24 severe cases. We identified 35 severity-associated genes and characterized their performance in predicting severity. The set of genes can be utilized as biomarkers in a prognostic test for risk stratification of COVID-19 patients in a clinical setting.

2. Data Sets

We used whole blood gene expression data collected from RNA-Seq of 62 COVID-19 patients enrolled prospectively with community-acquired lower respiratory tract infection by SARS-Cov-2 within the first 24 hours of hospital admission. The cohort contained non-severe (n=39) and severe disease groups (n=23, of which 6 died).

3. Methods

Data was processed with the Inflammatix internal pipeline using well established open source tools (FASTQC, STAR). We then used statistical package DESeq2 to both normalize the data and rank differentially expressed genes. DESeq2 is one of the most commonly used software packages specifically designed for identifying differentially expressed genes from RNA sequencing data. Briefly, it performs data normalization to account for sequencing and RNA composition biases, then estimates dispersion for each gene in each comparison group and uses this to fit negative binomial distribution. The significance of differences in gene expression is assessed using a Wald test statistic. We also used standardized effect size (Hedge's g), as criteria to further limit the number of genes. Hedges' g is a robust estimate of effect sizes as it accounts for variance, resulting in robust estimation of effect in even moderately sized cohorts.

4. Results

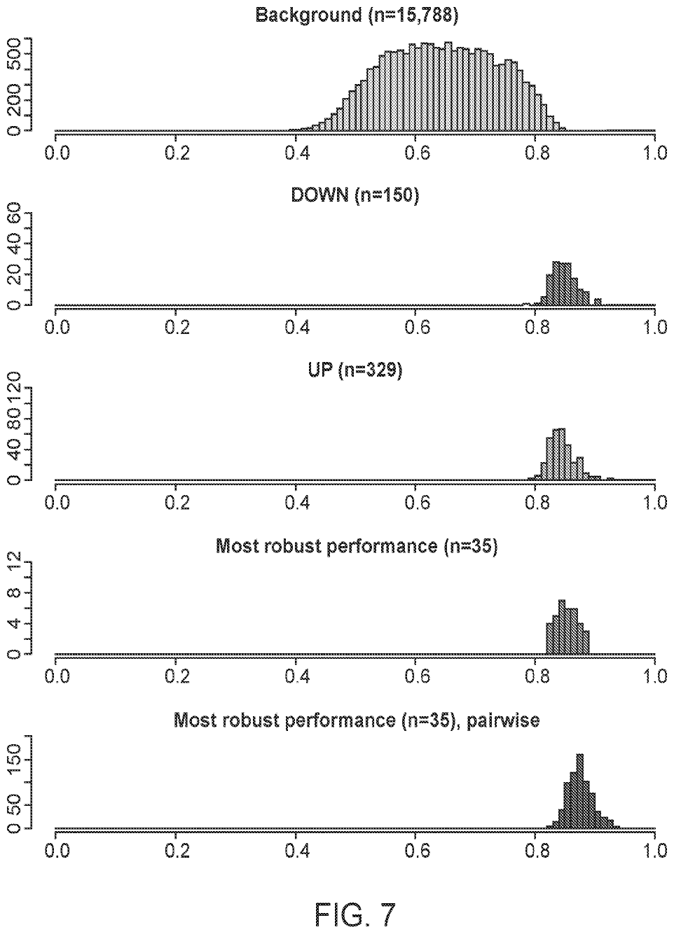

Differential expression was assessed at multiple threshold choices of fold change (FC), effect size (ES), and Benjamini-Hochberg corrected p-value (P-adjusted). At FC>1.5 and P-adjusted <0.05, a threshold that corresponds 80% power for even high heterogenicity, we identified 1,865 differentially expressed genes. This number is impractical for application development; therefore, to focus our effort on most applicable signal, we chose to use a more stringent cutoff at P-adjusted <0.005 and |ES|>1.3 (which is equivalent to FC of 2). At these thresholds, we identified 479 genes: 329 up- and 150 down-regulated in severe vs non-severe patients. To establish a background performance level, we first estimated gene-wise area under curve (AUC) of receiving operating curve (ROC) for all measured genes (FIG. 7A, AUC ranged from 0.36 to 0.87 with median of 0.64). AUC for the selected 479 genes ranged from 0.78-0.93, with the median of 0.84 (FIGS. 7B, 7C).

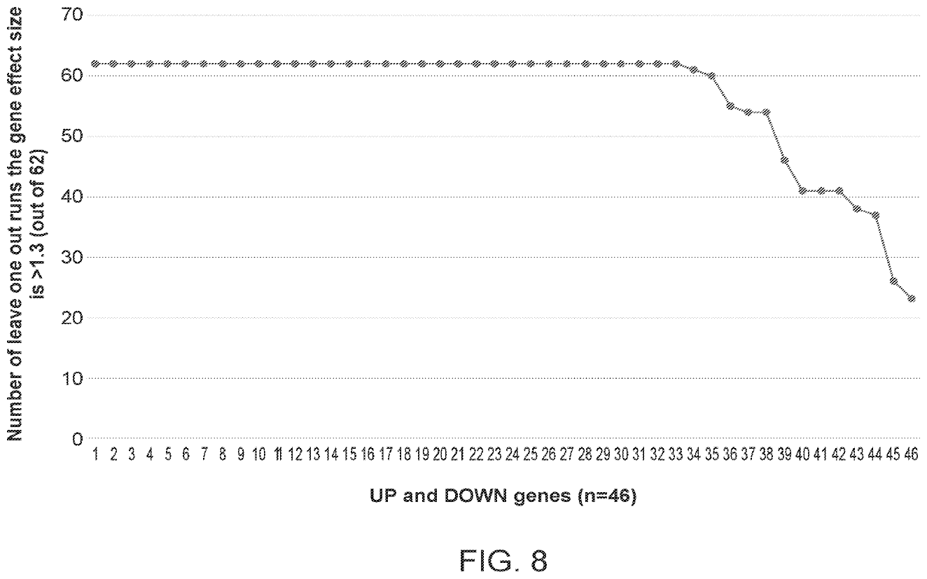

We then selected top 10% most highly expressed genes in the 329 up- and 150 down-regulated genes separately, resulting in 32 up- and 15 down-regulated genes, a total of 47 genes, as genes with higher expression often perform more robustly in our assay. We further narrowed down the list to 35 by keeping only genes present in 60 times or more out of 62 leave-one-out (LOO) gene selections (FIG. 8). Notably these genes represent the most robust selection in our data, 33 out of 35 genes are present in all possible 62 leave-one-out selections.

Individual AUCs for these 35 genes shown in FIG. 7D range from 0.82 to 0.89, with a median of 0.84 (see also Table 5). We also evaluated the performance of all 595 combinations of 2 genes out of the 35 genes and their AUCs are shown in FIG. 7E and Table 6. The difference-of-geometric-means score (over-expressed minus under-expressed) of 35 identified biomarker genes had the highest AUC (0.91, FIG. 8).

5. Discussion

COVID-19 is a rapidly evolving pandemic. To the best of our knowledge we are the first group to report RNA-seq gene expression of whole blood from a significant number of patients with diverse COVID-19 severity. These 62 samples allowed us to identify core set of genes that can potentially be used to predict COVID-19 severity, allowing for faster and more accurate triage of patients in a timely manner.

TABLE 5
Thirty-five genes with robust effect size in severe vs non-severe COVID-19 patients.
We used multiple filtering steps to narrow down our gene list to 35 most robustly
performing: a) Absolute effect size >1.3 and P-adjusted <0.005, 2) Top 10% of mean expression
and c) Robustness in leave one out analysis (Nes_1p3_loo).

| Ensmbl Gene ID | Gene Symbol | Mean expression | Effect Size | genelist1 | auc |
|---|---|---|---|---|---|
| ENSG00000168329 | CXC3R1 | 1826.780434 | −1.6910938 | DOWN | 0.88628763 |
| ENSG00000197629 | MPEG1 | 5269.490619 | −1.6350264 | DOWN | 0.88071349 |
| ENSG00000112062 | MAPK14 | 7268.52371 | 1.64525744 | UP | 0.87402453 |
| ENSG00000257335 | MGAM | 10683.16994 | 1.55698313 | UP | 0.86845039 |
| ENSG00000136040 | PLXNC1 | 11897.5858 | 1.56991196 | UP | 0.87513935 |
| ENSG00000113916 | BCL6 | 13833.59022 | 1.55803228 | UP | 0.87736901 |
| ENSG00000106780 | MEGF9 | 11246.30043 | 1.53273306 | UP | 0.85953177 |
| ENSG00000101265 | RASSF2 | 12346.41541 | 1.48688372 | UP | 0.87402453 |
| ENSG00000140199 | SLC12A6 | 6701.406003 | 1.52549454 | UP | 0.88071349 |
| ENSG00000100731 | PCNX1 | 8551.536171 | 1.53667248 | UP | 0.8606466 |
| ENSG00000162777 | DENND2D | 2025.899598 | −1.456647 | DOWN | 0.8483835 |
| ENSG00000188042 | CR1 | 7224.035539 | 1.4746745 | UP | 0.84503902 |
| ENSG00000134954 | ETS1 | 4105.330272 | −1.4879428 | DOWN | 0.85730212 |
| ENSG00000003402 | CFLAR | 19086.07732 | 1.45450612 | UP | 0.86510591 |
| ENSG00000163162 | RNF149 | 10690.52226 | 1.47251923 | UP | 0.8606466 |
| ENSG00000163947 | ARHGEF3 | 1685.838189 | −1.4055957 | DOWN | 0.86287625 |
| ENSG00000143226 | LRP10 | 8467.654298 | 1.39092562 | UP | 0.84726867 |
| ENSG00000151726 | GCA | 8040.910279 | 1.41533402 | UP | 0.83389075 |
| ENSG00000071054 | MAP4K4 | 8297.160023 | 1.40490525 | UP | 0.85172798 |
| ENSG00000203710 | EVL | 2264.423259 | −1.4355774 | DOWN | 0.84392419 |
| ENSG00000123066 | MED13L | 8510.802862 | 1.36471261 | UP | 0.85953177 |
| ENSG00000093072 | BASP1 | 7561.561554 | 1.3621833 | UP | 0.84169454 |
| ENSG00000186407 | CD300E | 3053.408879 | −1.4208448 | DOWN | 0.86399108 |
| ENSG00000010810 | FYN | 2652.221965 | −1.4203203 | DOWN | 0.85061315 |
| ENSG00000176788 | SOD2 | 13047.3128 | 1.38793635 | UP | 0.8361204 |
| ENSG00000168685 | MCTP2 | 8605.960049 | 1.38661521 | UP | 0.82720178 |
| ENSG00000196405 | ACSL1 | 21558.56451 | 1.36061687 | UP | 0.84057971 |
| ENSG00000112096 | VNN2 | 9259.50726 | 1.35486138 | UP | 0.8238573 |
| ENSG00000245164 | LINC00861 | 2246.040458 | −1.4142383 | DOWN | 0.85730212 |
| ENSG00000180644 | SLC2A3 | 8628.796852 | 1.36341638 | UP | 0.82608696 |
| ENSG00000122862 | TRAC | 1737.258134 | −1.3750032 | DOWN | 0.82943144 |
| ENSG00000197324 | ARL4C | 1674.913726 | −1.3975753 | DOWN | 0.84615385 |
| ENSG00000170006 | IPRF1 | 2312.14155 | −1.3792383 | DOWN | 0.83835006 |
| ENSG00000103569 | IL7R | 5596.262319 | −1.3524564 | DOWN | 0.83835006 |
| ENSG00000135905 | SRGN | 14449.19906 | 1.35268161 | UP | 0.83946488 |

TABLE 6
All two-gene combinations of the 35 gene set, and their performance characteristics
across the COVID dataset. All AUCs above 0.85 are potentially clinically useful.

| Symbol_gene_1 | Symbol_gene_2 | AUC | Symbol_gene_1 | Symbol_gene_2 | AUC | Symbol_gene_1 | Symbol_gene_2 | AUC |
|---|---|---|---|---|---|---|---|---|
| RASSF2 | FYN | 0.883 | RASSF2 | MED13L | 0.866 | RASSF2 | DENND2D | 0.884 |
| SOD2 | FYN | 0.854 | MAP4K4 | ETS1 | 0.88 | MED13L | DENND2D | 0.873 |
| RNF149 | FYN | 0.889 | RASSF2 | ETS1 | 0.884 | SLC12A6 | DENND2D | 0.89 |
| MGAM | FYN | 0.872 | SOD2 | ETS1 | 0.867 | ARL4C | DENND2D | 0.891 |
| MAP4K4 | FYN | 0.88 | PCNX1 | ETS1 | 0.88 | ETS1 | DENND2D | 0.885 |
| ADA2 | FYN | 0.856 | ADA2 | ETS1 | 0.856 | BCL6 | DENND2D | 0.886 |
| PCNX1 | FYN | 0.866 | AQP9 | ETS1 | 0.857 | GCA | DENND2D | 0.889 |
| TRAC | FYN | 0.863 | MGAM | ETS1 | 0.88 | VNN2 | RNF149 | 0.851 |
| MAPK14 | FYN | 0.881 | EIF4G2 | ETS1 | 0.855 | RASSF2 | RNF149 | 0.886 |
| MEGF9 | FYN | 0.894 | PLXNC1 | ETS1 | 0.883 | AQP9 | RNF149 | 0.839 |
| AQP9 | FYN | 0.849 | FYN | ETS1 | 0.874 | SLC12A6 | RNF149 | 0.878 |
| SLC12A6 | FYN | 0.884 | MAPK14 | ETS1 | 0.883 | MED13L | RNF149 | 0.873 |
| CFLAR | FYN | 0.866 | GCA | ETS1 | 0.864 | EIF4G2 | RNF149 | 0.852 |
| PLXNC1 | FYN | 0.878 | CAP1 | ETS1 | 0.866 | PCNX1 | RNF149 | 0.878 |
| EIF4G2 | FYN | 0.864 | CFLAR | ETS1 | 0.876 | BCL6 | RNF149 | 0.885 |
| ARL4C | FYN | 0.886 | MEGF9 | ETS1 | 0.875 | CAP1 | RNF149 | 0.855 |
| GCA | FYN | 0.857 | EVL | ETS1 | 0.872 | MEGF9 | RNF149 | 0.878 |
| VNN2 | FYN | 0.88 | TRAC | ETS1 | 0.865 | CFLAR | RNF149 | 0.871 |
| CAP1 | FYN | 0.864 | RNF149 | ETS1 | 0.878 | TRAC | RNF149 | 0.856 |
| EVL | FYN | 0.872 | MED13L | ETS1 | 0.875 | ADA2 | RNF149 | 0.849 |
| MED13L | FYN | 0.876 | SLC12A6 | ETS1 | 0.878 | PLXNC1 | RNF149 | 0.881 |
| BCL6 | FYN | 0.88 | BCL6 | ETS1 | 0.884 | GCA | RNF149 | 0.853 |
| CFLAR | AQP9 | 0.843 | ARL4C | ETS1 | 0.89 | MAPK14 | RNF149 | 0.874 |
| CFLAR | MAP4K4 | 0.868 | VNN2 | ETS1 | 0.878 | MAP4K4 | RNF149 | 0.872 |
| AQP9 | MAP4K4 | 0.845 | TRAC | PLXNC1 | 0.875 | CFLAR | ARHGEF3 | 0.89 |
| AQP9 | PCNX1 | 0.841 | AQP9 | PLXNC1 | 0.847 | DENND2D | ARHGEF3 | 0.87 |
| MAP4K4 | PCNX1 | 0.872 | EIF4G2 | PLXNC1 | 0.867 | EVL | ARHGEF3 | 0.873 |
| CFLAR | PCNX1 | 0.864 | BCL6 | PLXNC1 | 0.875 | SOD2 | ARHGEF3 | 0.865 |
| AQP9 | RASSF2 | 0.863 | MEGF9 | PLXNC1 | 0.89 | ARL4C | ARHGEF3 | 0.899 |
| CFLAR | RASSF2 | 0.884 | MAPK14 | PLXNC1 | 0.875 | TRAC | ARHGEF3 | 0.87 |
| PCNX1 | RASSF2 | 0.882 | MAP4K4 | PLXNC1 | 0.873 | EIF4G2 | ARHGEF3 | 0.875 |
| MAP4K4 | RASSF2 | 0.87 | MED13L | PLXNC1 | 0.883 | ADA2 | ARHGEF3 | 0.883 |
| CFLAR | MEGF9 | 0.886 | CFLAR | PLXNC1 | 0.88 | VNN2 | ARHGEF3 | 0.881 |
| MAP4K4 | MEGF9 | 0.871 | VNN2 | PLXNC1 | 0.872 | MAP4K4 | ARHGEF3 | 0.882 |
| AQP9 | MEGF9 | 0.855 | CAP1 | PLXNC1 | 0.872 | MGAM | ARHGEF3 | 0.88 |
| PCNX1 | MEGF9 | 0.886 | PCNX1 | PLXNC1 | 0.878 | PLXNC1 | ARHGEF3 | 0.902 |
| RASSF2 | MEGF9 | 0.868 | RASSF2 | PLXNC1 | 0.89 | SLC12A6 | ARHGEF3 | 0.902 |
| MAP4K4 | MAPK14 | 0.872 | MEGF9 | SLC12A6 | 0.877 | FYN | ARHGEF3 | 0.871 |
| AQP9 | MAPK14 | 0.856 | MAPK14 | SLC12A6 | 0.884 | PCNX1 | ARHGEF3 | 0.9 |
| PCNX1 | MAPK14 | 0.871 | CFLAR | SLC12A6 | 0.876 | GCA | ARHGEF3 | 0.877 |
| CFLAR | MAPK14 | 0.873 | AQP9 | SLC12A6 | 0.856 | CAP1 | ARHGEF3 | 0.882 |
| MEGF9 | MAPK14 | 0.896 | CAP1 | SLC12A6 | 0.864 | MEGF9 | ARHGEF3 | 0.891 |
| RASSF2 | MAPK14 | 0.88 | BCL6 | SLC12A6 | 0.88 | MAPK14 | ARHGEF3 | 0.895 |
| MEGF9 | VNN2 | 0.874 | VNN2 | SLC12A6 | 0.866 | AQP9 | ARHGEF3 | 0.876 |
| MAP4K4 | VNN2 | 0.855 | MAP4K4 | SLC12A6 | 0.876 | BCL6 | ARHGEF3 | 0.886 |
| RASSF2 | VNN2 | 0.874 | TRAC | SLC12A6 | 0.876 | MED13L | ARHGEF3 | 0.892 |
| MAPK14 | VNN2 | 0.88 | MED13L | SLC12A6 | 0.878 | RNF149 | ARHGEF3 | 0.901 |
| AQP9 | VNN2 | 0.843 | PLXNC1 | SLC12A6 | 0.889 | RASSF2 | ARHGEF3 | 0.882 |
| CFLAR | VNN2 | 0.867 | PCNX1 | SLC12A6 | 0.881 | ETS1 | ARHGEF3 | 0.896 |
| PCNX1 | VNN2 | 0.88 | RASSF2 | SLC12A6 | 0.887 | ADA2 | CXC3R1 | 0.9 |
| RASSF2 | CAP1 | 0.875 | EIF4G2 | SLC12A6 | 0.861 | MGAM | CXC3R1 | 0.9 |
| AQP9 | CAP1 | 0.845 | MEGF9 | ADA2 | 0.86 | AQP9 | CXC3R1 | 0.885 |
| VNN2 | CAP1 | 0.856 | AQP9 | ADA2 | 0.827 | SOD2 | CXC3R1 | 0.889 |
| MAP4K4 | CAP1 | 0.871 | MAPK14 | ADA2 | 0.857 | PCNX1 | CXC3R1 | 0.907 |
| MAPK14 | CAP1 | 0.87 | CAP1 | ADA2 | 0.835 | SLC12A6 | CXC3R1 | 0.913 |
| PCNX1 | CAP1 | 0.86 | 3BCL6 | ADA2 | 0.87 | DENND2D | CXC3R1 | 0.9 |
| MEGF9 | CAP1 | 0.862 | VNN2 | ADA2 | 0.85 | 6MAPK14 | CXC3R1 | 0.902 |
| CFLAR | CAP1 | 0.857 | TRAC | ADA2 | 0.838 | MAP4K4 | CXC3R1 | 0.901 |
| MAP4K4 | BCL6 | 0.873 | MAP4K4 | ADA2 | 0.855 | EVL | CXC3R1 | 0.896 |
| RASSF2 | BCL6 | 0.883 | MED13L | ADA2 | 0.848 | ARHGEF3 | CXC3R1 | 0.896 |
| PCNX1 | BCL6 | 0.881 | PLXNC1 | ADA2 | 0.857 | FYN | CXC3R1 | 0.883 |
| MEGF9 | BCL6 | 0.892 | PCNX1 | ADA2 | 0.843 | PLXNC1 | CXC3R1 | 0.913 |
| VNN2 | BCL6 | 0.886 | CFLAR | ADA2 | 0.845 | BCL6 | CXC3R1 | 0.901 |
| CAP1 | BCL6 | 0.889 | RASSF2 | ADA2 | 0.87 | ETS1 | CXC3R1 | 0.914 |
| MAPK14 | BCL6 | 0.876 | EIF4G2 | ADA2 | 0.837 | RNF149 | CXC3R1 | 0.916 |
| AQP9 | BCL6 | 0.86 | SLC12A6 | ADA2 | 0.855 | ARL4C | CXC3R1 | 0.92 |
| CFLAR | BCL6 | 0.883 | MEGF9 | GCA | 0.865 | TRAC | CXC3R1 | 0.889 |
| PCNX1 | EIF4G2 | 0.857 | MED13L | GCA | 0.848 | MEGF9 | CXC3R1 | 0.919 |
| CAP1 | EIF4G2 | 0.843 | RASSF2 | GCA | 0.873 | RASSF2 | CXC3R1 | 0.960 |
| MAPK14 | EIF4G2 | 0.864 | VNN2 | GCA | 0.861 | EIF4G2 | CXC3R1 | 0.895 |
| CFLAR | EIF4G2 | 0.856 | SLC12A6 | GCA | 0.862 | MED13L | CXC3R1 | 0.909 |
| VNN2 | EIF4G2 | 0.852 | PLXNC1 | GCA | 0.86 | CAP1 | CXC3R1 | 0.89 |
| RASSF2 | EIF4G2 | 0.873 | MAP4K4 | GCA | 0.852 | GCA | CXC3R1 | 0.894 |
| BCL6 | EIF4G2 | 0.882 | BCL6 | GCA | 0.865 | VNN2 | CXC3R1 | 0.901 |
| AQP9 | EIF4G2 | 0.848 | PCNX1 | GCA | 0.853 | CFLAR | CXC3R1 | 0.909 |
| MAP4K4 | EIF4G2 | 0.873 | CFLAR | GCA | 0.851 | MGAM | MCTP2 | 0.875 |
| MEGF9 | EIF4G2 | 0.864 | TRAC | GCA | 0.853 | SLC12A6 | MCTP2 | 0.871 |
| PCNX1 | TRAC | 0.873 | CAP1 | GCA | 0.842 | MAP4K4 | MCTP2 | 0.868 |
| VNN2 | TRAC | 0.853 | MAPK14 | GCA | 0.858 | AQP9 | MCTP2 | 0.868 |
| RASSF2 | TRAC | 0.877 | ADA2 | GCA | 0.835 | DENND2D | MCTP2 | 0.861 |
| CFLAR | TRAC | 0.865 | EIF4G2 | GCA | 0.841 | CXC3R1 | MCTP2 | 0.895 |
| AQP9 | TRAC | 0.848 | AQP9 | GCA | 0.839 | ARHGEF3 | MCTP2 | 0.885 |
| BCL6 | TRAC | 0.887 | SOD2 | DENND2D | 0.862 | ADA2 | MCTP2 | 0.873 |
| MAPK14 | TRAC | 0.875 | PCNX1 | DENND2D | 0.9 | FYN | MCTP2 | 0.861 |
| MAP4K4 | TRAC | 0.876 | MAP4K4 | DENND2D | 0.884 | TRAC | MCTP2 | 0.874 |
| EIF4G2 | TRAC | 0.838 | MGAM | DENND2D | 0.891 | CAP1 | MCTP2 | 0.882 |
| MEGF9 | TRAC | 0.864 | TRAC | DENND2D | 0.874 | RASSF2 | MCTP2 | 0.868 |
| CAP1 | TRAC | 0.841 | MEGF9 | DENND2D | 0.893 | RNF149 | MCTP2 | 0.88 |
| AQP9 | MED13L | 0.836 | FYN | DENND2D | 0.878 | EVL | MCTP2 | 0.872 |
| CFLAR | MED13L | 0.868 | ADA2 | DENND2D | 0.885 | ETS1 | MCTP2 | 0.863 |
| MAP4K4 | MED13L | 0.856 | VNN2 | DENND2D | 0.864 | BCL6 | MCTP2 | 0.88 |
| TRAC | MED13L | 0.866 | EIF4G2 | DENND2D | 0.876 | MEGF9 | MCTP2 | 0.885 |
| EIF4G2 | MED13L | 0.861 | CAP1 | DENND2D | 0.88 | VNN2 | MCTP2 | 0.877 |
| PCNX1 | MED13L | 0.877 | PLXNC1 | DENND2D | 0.894 | MAPK14 | MCTP2 | 0.881 |
| MEGF9 | MED13L | 0.872 | RNF149 | DENND2D | 0.889 | PLXNC1 | MCTP2 | 0.88 |
| BCL6 | MED13L | 0.874 | MAPK14 | DENND2D | 0.897 | CFLAR | MCTP2 | 0.876 |
| VNN2 | MED13L | 0.852 | EVL | DENND2D | 0.88 | EIF4G2 | MCTP2 | 0.89 |
| MAPK14 | MED13L | 0.88 | AQP9 | DENND2D | 0.877 | SOD2 | MCTP2 | 0.858 |
| CAP1 | MED13L | 0.866 | CFLAR | DENND2D | 0.893 | GCA | MCTP2 | 0.877 |
| MED13L | MCTP2 | 0.853 | EVL | CR1 | 0.871 | TRAC | EVL | 0.864 |
| ARL4C | MCTP2 | 0.87 | FYN | CR1 | 0.854 | EIF4G2 | EVL | 0.856 |
| PCNX1 | MCTP2 | 0.872 | MAPK14 | CR1 | 0.88 | SOD2 | EVL | 0.857 |
| EIF4G2 | SOD2 | 0.871 | GCA | CR1 | 0.873 | CAP1 | EVL | 0.867 |
| SLC12A6 | SOD2 | 0.863 | EIF4G2 | CR1 | 0.868 | VNN2 | EVL | 0.87 |
| CFLAR | SOD2 | 0.855 | VNN2 | CR1 | 0.872 | ARL4C | EVL | 0.866 |
| PCNX1 | SOD2 | 0.856 | ARL4C | CR1 | 0.883 | BCL6 | EVL | 0.856 |
| VNN2 | SOD2 | 0.845 | MAP4K4 | CR1 | 0.871 | PCNX1 | EVL | 0.86 |
| CAP1 | SOD2 | 0.873 | AQP9 | CR1 | 0.856 | SLC12A6 | EVL | 0.872 |
| MEGF9 | SOD2 | 0.858 | SLC12A6 | ACSL1 | 0.873 | MEGF9 | EVL | 0.882 |
| AQP9 | SOD2 | 0.841 | CXC3R1 | ACSL1 | 0.9 | MAPK14 | EVL | 0.868 |
| BCL6 | SOD2 | 0.865 | CFLAR | ACSL1 | 0.874 | MED13L | EVL | 0.857 |
| PLXNC1 | SOD2 | 0.855 | CAP1 | ACSL1 | 0.855 | MCTP2 | LINC00861 | 0.855 |
| MAP4K4 | SOD2 | 0.849 | RASSF2 | ACSL1 | 0.873 | CXC3R1 | LINC00861 | 0.895 |
| RNF149 | SOD2 | 0.857 | TRAC | ACSL1 | 0.849 | CAP1 | LINC00861 | 0.894 |
| GCA | SOD2 | 0.853 | DENND2D | ACSL1 | 0.864 | PLXNC1 | LINC00861 | 0.876 |
| RASSF2 | SOD2 | 0.856 | FYN | ACSL1 | 0.862 | FYN | LINC00861 | 0.87 |
| MED13L | SOD2 | 0.846 | CD300E | ACSL1 | 0.906 | GCA | LINC00861 | 0.873 |
| TRAC | SOD2 | 0.865 | MAPK14 | ACSL1 | 0.878 | EIF4G2 | LINC00861 | 0.889 |
| ADA2 | SOD2 | 0.845 | BCL6 | ACSL1 | 0.9 | VNN2 | LINC00861 | 0.882 |
| MAPK14 | SOD2 | 0.861 | SOD2 | ACSL1 | 0.873 | SLC2A3 | LINC00861 | 0.903 |
| ARHGEF3 | SLC2A3 | 0.866 | CR1 | ACSL1 | 0.871 | ACSL1 | LINC00861 | 0.88 |
| AQP9 | SLC2A3 | 0.893 | AQP9 | ACSL1 | 0.854 | ARL4C | LINC00861 | 0.868 |
| MGAM | SLC2A3 | 0.9 | VNN2 | ACSL1 | 0.866 | MGAM | LINC00861 | 0.876 |
| SLC12A6 | SLC2A3 | 0.893 | ETS1 | ACSL1 | 0.853 | EVL | LINC00861 | 0.874 |
| RASSF2 | SLC2A3 | 0.891 | MED13L | ACSL1 | 0.865 | CD300E | LINC00861 | 0.919 |
| CXC3R1 | SLC2A3 | 0.876 | PLXNC1 | ACSL1 | 0.873 | ADA2 | LINC00861 | 0.874 |
| MAP4K4 | SLC2A3 | 0.893 | ADA2 | ACSL1 | 0.857 | RNF149 | LINC00861 | 0.894 |
| ETS1 | SLC2A3 | 0.885 | EVL | ACSL1 | 0.866 | SOD2 | LINC00861 | 0.855 |
| DENND2D | SLC2A3 | 0.858 | MCTP2 | ACSL1 | 0.871 | BCL6 | LINC00861 | 0.877 |
| MAPK14 | SLC2A3 | 0.9 | GCA | ACSL1 | 0.861 | SLC12A6 | LINC00861 | 0.877 |
| PLXNC1 | SLC2A3 | 0.909 | RNF149 | ACSL1 | 0.867 | MEGF9 | LINC00861 | 0.883 |
| ADA2 | SLC2A3 | 0.899 | EIF4G2 | ACSL1 | 0.855 | AQP9 | LINC00861 | 0.862 |
| EIF4G2 | SLC2A3 | 0.876 | MEGF9 | ACSL1 | 0.867 | MED13L | LINC00861 | 0.862 |
| FYN | SLC2A3 | 0.861 | ARL4C | ACSL1 | 0.897 | CR1 | LINC00861 | 0.858 |
| GCA | SLC2A3 | 0.899 | SLC2A3 | ACSL1 | 0.874 | TRAC | LINC00861 | 0.889 |
| BCL6 | SLC2A3 | 0.9 | ARHGEF3 | ACSL1 | 0.883 | ARHGEF3 | LINC00861 | 0.891 |
| MEGF9 | SLC2A3 | 0.906 | MGAM | ACSL1 | 0.892 | MAP4K4 | LINC00861 | 0.864 |
| RNF149 | SLC2A3 | 0.913 | PCNX1 | ACSL1 | 0.875 | MPEG1 | LINC00861 | 0.921 |
| ARL4C | SLC2A3 | 0.912 | MAP4K4 | ACSL1 | 0.867 | ETS1 | LINC00861 | 0.877 |
| TRAC | SLC2A3 | 0.876 | BCL6 | ARL4C | 0.885 | CFLAR | LINC00861 | 0.873 |
| CAP1 | SLC2A3 | 0.868 | EIF4G2 | ARL4C | 0.884 | PCNX1 | LINC00861 | 0.875 |
| CFLAR | SLC2A3 | 0.901 | AQP9 | ARL4C | 0.856 | RASSF2 | LINC00861 | 0.877 |
| VNN2 | SLC2A3 | 0.896 | CFLAR | ARL4C | 0.88 | DENND2D | LINC00861 | 0.878 |
| EVL | SLC2A3 | 0.9 | RNF149 | ARL4C | 0.867 | MAPK14 | LINC00861 | 0.878 |
| MCTP2 | SLC2A3 | 0.896 | MAP4K4 | ARL4C | 0.861 | CFLAR | MGAM | 0.872 |
| SOD2 | SLC2A3 | 0.894 | MEGF9 | ARL4C | 0.886 | GCA | MGAM | 0.87 |
| PCNX1 | SLC2A3 | 0.903 | VNN2 | ARL4C | 0.862 | PLXNC1 | MGAM | 0.876 |
| MED13L | SLC2A3 | 0.886 | GCA | ARL4C | 0.865 | RNF149 | MGAM | 0.89 |
| DENND2D | CD300E | 0.882 | PLXNC1 | ARL4C | 0.88 | RASSF2 | MGAM | 0.884 |
| SLC12A6 | CD300E | 0.912 | SLC12A6 | ARL4C | 0.886 | TRAC | MGAM | 0.896 |
| MAPK14 | CD300E | 0.915 | PCNX1 | ARL4C | 0.889 | EVL | MGAM | 0.865 |
| MAP4K4 | CD300E | 0.896 | RASSF2 | ARL4C | 0.877 | MAP4K4 | MGAM | 0.861 |
| RNF149 | CD300E | 0.912 | SOD2 | ARL4C | 0.852 | EIF4G2 | MGAM | 0.894 |
| CXC3R1 | CD300E | 0.903 | TRAC | ARL4C | 0.89 | SOD2 | MGAM | 0.858 |
| CFLAR | CD300E | 0.91 | ADA2 | ARL4C | 0.881 | ADA2 | MGAM | 0.865 |
| ARHGEF3 | CD300E | 0.895 | CAP1 | ARL4C | 0.884 | MAPK14 | MGAM | 0.875 |
| MGAM | CD300E | 0.9 | MED13L | ARL4C | 0.858 | MEGF9 | MGAM | 0.892 |
| TRAC | CD300E | 0.894 | MAPK14 | ARL4C | 0.887 | AQP9 | MGAM | 0.858 |
| ADA2 | CD300E | 0.894 | CD300E | MPEG1 | 0.881 | PCNX1 | MGAM | 0.876 |
| SOD2 | CD300E | 0.895 | CR1 | MPEG1 | 0.907 | ARL4C | MGAM | 0.884 |
| EIF4G2 | CD300E | 0.903 | CFLAR | MPEG1 | 0.91 | MED13L | MGAM | 0.88 |
| GCA | CD300E | 0.895 | SLC2A3 | MPEG1 | 0.887 | CAP1 | MGAM | 0.894 |
| RASSF2 | CD300E | 0.909 | SLC12A6 | MPEG1 | 0.928 | BCL6 | MGAM | 0.873 |
| BCL6 | CD300E | 0.905 | FYN | MPEG1 | 0.906 | VNN2 | MGAM | 0.884 |
| ETS1 | CD300E | 0.925 | PLXNC1 | MPEG1 | 0.922 | SLC12A6 | MGAM | 0.88 |
| MED13L | CD300E | 0.899 | MCTP2 | MPEG1 | 0.921 | VNN2 | FCGR2A | 0.854 |
| VNN2 | CD300E | 0.906 | BCL6 | MPEG1 | 0.897 | MCTP2 | FCGR2A | 0.854 |
| CAP1 | CD300E | 0.897 | GCA | MPEG1 | 0.882 | DENND2D | FCGR2A | 0.866 |
| AQP9 | CD300E | 0.903 | CXC3R1 | MPEG1 | 0.912 | MEGF9 | FCGR2A | 0.861 |
| FYN | CD300E | 0.889 | CAP1 | MPEG1 | 0.897 | CXC3R1 | FCGR2A | 0.897 |
| PCNX1 | CD300E | 0.906 | MED13L | MPEG1 | 0.924 | CAP1 | FCGR2A | 0.858 |
| PLXNC1 | CD300E | 0.913 | ETS1 | MPEG1 | 0.931 | ADA2 | FCGR2A | 0.844 |
| MCTP2 | CD300E | 0.924 | DENND2D | MPEG1 | 0.929 | PLXNC1 | FCGR2A | 0.87 |
| MEGF9 | CD300E | 0.924 | MAP4K4 | MPEG1 | 0.922 | FYN | FCGR2A | 0.844 |
| ARL4C | CD300E | 0.914 | MGAM | MPEG1 | 0.905 | ARHGEF3 | FCGR2A | 0.882 |
| SLC2A3 | CD300E | 0.868 | EVL | MPEG1 | 0.896 | SOD2 | FCGR2A | 0.854 |
| EVL | CD300E | 0.903 | PCNX1 | MPEG1 | 0.92 | EIF4G2 | FCGR2A | 0.857 |
| DENND2D | CR1 | 0.86 | ACSL1 | MPEG1 | 0.92 | BCL6 | FCGR2A | 0.87 |
| TRAC | CR1 | 0.855 | EIF4G2 | MPEG1 | 0.893 | RNF149 | FCGR2A | 0.863 |
| RASSF2 | CR1 | 0.865 | AQP9 | MPEG1 | 0.882 | AQP9 | FCGR2A | 0.846 |
| PCNX1 | CR1 | 0.867 | ARL4C | MPEG1 | 0.921 | MED13L | FCGR2A | 0.838 |
| SOD2 | CR1 | 0.845 | VNN2 | MPEG1 | 0.914 | ARL4C | FCGR2A | 0.873 |
| MED13L | CR1 | 0.863 | ARHGEF3 | MPEG1 | 0.921 | RASSF2 | FCGR2A | 0.856 |
| ADA2 | CR1 | 0.863 | MAPK14 | MPEG1 | 0.902 | TRAC | FCGR2A | 0.86 |
| CXC3R1 | CR1 | 0.876 | ADA2 | MPEG1 | 0.887 | EVL | FCGR2A | 0.857 |
| SLC12A6 | CR1 | 0.874 | RNF149 | MPEG1 | 0.924 | LINC00861 | FCGR2A | 0.846 |
| MCTP2 | CR1 | 0.857 | SOD2 | MPEG1 | 0.892 | CD300E | FCGR2A | 0.913 |
| CFLAR | CR1 | 0.868 | MEGF9 | MPEG1 | 0.936 | CFLAR | FCGR2A | 0.849 |
| CD300E | CR1 | 0.895 | RASSF2 | MPEG1 | 0.936 | PCNX1 | FCGR2A | 0.861 |
| CAP1 | CR1 | 0.871 | TRAC | MPEG1 | 0.895 | GCA | FCGR2A | 0.853 |
| BCL6 | CR1 | 0.874 | CFLAR | EVL | 0.869 | CR1 | FCGR2A | 0.854 |
| ETS1 | CR1 | 0.864 | AQP9 | EVL | 0.837 | ETS1 | FCGR2A | 0.851 |
| ARHGEF3 | CR1 | 0.871 | GCA | EVL | 0.851 | SLC12A6 | FCGR2A | 0.856 |
| RNF149 | CR1 | 0.887 | PLXNC1 | EVL | 0.861 | ACSL1 | FCGR2A | 0.849 |
| SLC2A3 | CR1 | 0.864 | MAP4K4 | EVL | 0.855 | SLC2A3 | FCGR2A | 0.884 |
| MEGF9 | CR1 | 0.876 | ADA2 | EVL | 0.846 | MAPK14 | FCGR2A | 0.874 |
| PLXNC1 | CR1 | 0.881 | RNF149 | EVL | 0.865 | MPEG1 | FCGR2A | 0.906 |
| MGAM | CR1 | 0.871 | RASSF2 | EVL | 0.875 | MAP4K4 | FCGR2A | 0.863 |

### E. Example 5. A 6-mRNA Host Response Whole-Blood Classifier Trained Using Patients with Non-COVID-19 Viral Infections Accurately Predicts Severity of COVID-19

1. Introduction

Based on previous results that there is a shared blood host-immune response-based mRNA prognostic signature among patients with acute viral infections, we hypothesized that a parsimonious, clinically translatable gene signature for predicting outcome in patients with viral infection can be identified. We tested this hypothesis by integrating 21 independent data sets with 705 peripheral blood transcriptome profiles from patients with acute viral infections and identified a 6-mRNA host-response-based signature for mortality prediction across these multiple viral datasets. Next, we validated the locked model in 21 independent retrospective cohorts of 1,417 blood transcriptome profiles of patients with a variety of viral infections (non-COVID). Next, we validated our 6-mRNA model in an independent prospectively collected cohort of patients with COVID-19, showing an ability to predict outcomes despite having been entirely trained using non-COVID data. Our results suggest there is a conserved host response associated with outcomes in acute viral infections. Finally, we showed validity of a rapid isothermal version of the 6-mRNA host-response-signature which is being further developed into a rapid molecular test (CoVerity™) to assist in improving management of patients with COVID-19 and other acute viral infections.

2. Materials and Methods

### Data Collection, Curation, And Sample Labeling

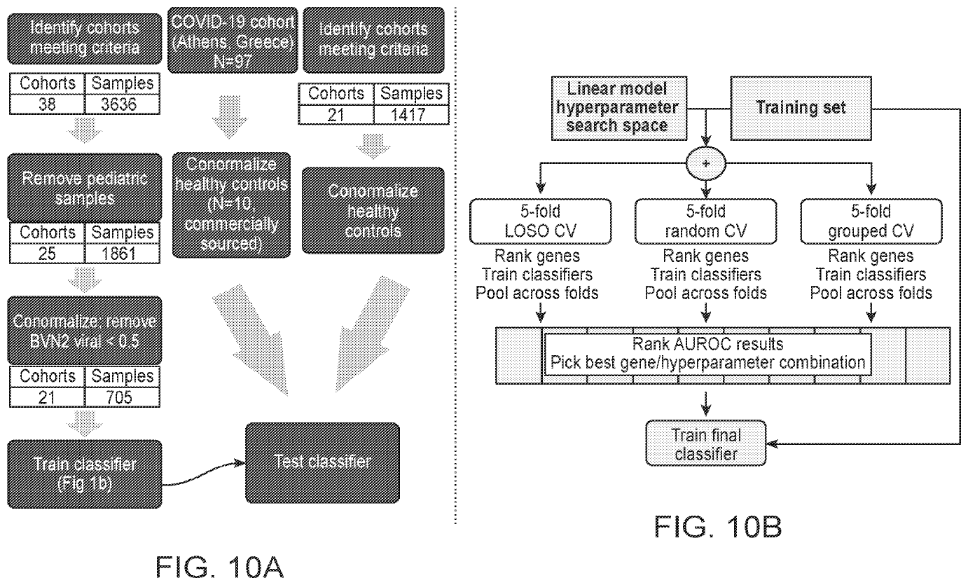

We searched public repositories (NCBI GEO and EBI ArrayExpress) for studies of typical acute infection with mortality data present. After removal of pediatric and entirely non-viral datasets, we identified 17 microarray or RNAseq peripheral blood acute infection studies composed of samples from 1,861 adult patients with either 28-day or 30-day mortality information (FIG. 10 and Table 7). We processed and co-normalized these datasets as previously described (19).

The number of cases with clinically adjudicated viral infection and known mortality outcome among the public samples was too low for robust modeling. Thus, to increase the number of training samples, we assigned viral infection status using a previously developed gene-expression-based bacterial/viral classifier, whose accuracy approaches that of clinical adjudication. Specifically, we utilized an updated version of our previously described neural network-based classifier for diagnosis of bacterial vs. viral infections called ‘Inflammatix Bacterial-Viral Noninfected version 2’ (IMX-BVN-2), (18). The idea is that this method would increase the number of mortality samples with viral infection, without introducing many false positives. For all samples, we applied IMX-BVN-2 to assign a probability of bacterial or viral infection and retained samples for which viral probability according to IMX-BVN-2 was ≥0.5. We refer to this assessment of viral infection as computer-aided adjudication. Out of 1,861 samples, we found 311 samples which had IMX-BVN-2 probability of viral infection ≥0.5, of which 9 patients died within 30-day period.

In addition to this public microarray/RNAseq data, we included 394 samples across 4 independent cohorts (19) that were profiled using NanoString nCounter, of which 14 patients died (Table 7). Thus, overall we included 705 blood samples across 21 independent studies from patients with computer aided-adjudication of viral infection and short-term mortality outcome. Importantly, none of these patients had SARS-CoV-2 infection as they were all enrolled prior to November 2019.

### Selection of Variables for Classifier Development

We preselected 29 mRNAs from which to develop the classifier for several biological and practical reasons. Biologically, the 29 mRNAs are composed of an 11-gene set for predicting 30-day mortality in critically ill patients and a repeatedly validated 18-gene set that can identify viral vs bacterial or noninfectious inflammation (17-19). Thus, we hypothesized that if a generalizable viral severity signature were possible, we likely had appropriate (and pre-vetted) variables here. By limiting our input variables, we also lowered our risk of overfitting to the training data. From a practical perspective, first, we are developing a point-of-care diagnostic platform for measuring these 29 genes in less than 30 minutes. A classifier developed using a subset of these 29 genes would allow us to develop a rapid point-of-care test on our existing platform. Second, 4 of the 21 cohorts included in the training were Inflammatix studies that profiled these 29 genes using NanoString nCounter and therefore for those studies this was the only mRNA expression data available.

### Development of a Classifier Using Machine Learning

We analyzed the 705 viral samples using cross-validation (CV) for ranking and selecting machine learning classifiers. We explored three variants of cross-validation: (1) 5-fold random CV. (2) 5-fold grouped CV, where each fold comprises multiple studies, and each study is assigned to exactly one CV fold, and (3) leave-one-study-out (LOSO), where each study forms a CV fold. We included non-random CV variants because we recently demonstrated that the leave-one-study-out cross-validation may reduce overfitting during training and produce more robust classifiers, for certain datasets (19). The hyperparameter search space was based on machine learning best practices and our previous results in model optimization in infectious disease diagnostics (21). For rapid turnaround and to reduce overfitting, we only investigated linear classifiers (support vector machine with linear kernel, logistic regression, and multi-layer perceptron with linear activation function) and limited the number of hyperparameter configurations we searched to 1000 per classifier. Finally, to ensure a parsimonious signature for translation to a rapid molecular assay, we limited the number of genes in the final model to six. To select the six genes, we applied forward selection and univariate feature ranking. We followed best practices to avoid overfitting in the gene selection process (22, 23).

We performed cross-validations for each of the hyperparameter configurations. Within each fold, we sorted the absolute value of the genes' Pearson correlation with class label (survived/died). We then trained a classifier using the six top-ranked genes and applied it to the left-out fold. The predicted probabilities from the folds were pooled, and the Area Under a Receiver Operating Characteristic (AUROC) curve over the pooled cross-validation probabilities was used as a metric to rank classification models. The final ranking of genes was determined using average ranking across the CV folds. Once the best-ranking model hyperparameters were selected and the final list of six genes was established, the final model was trained using the entire training set and the ‘locked’ hyperparameters. The corresponding model weights were locked and the final classifier was then tested in an independent prospective cohort of patients with COVID-19, and in independent retrospective cohort of patients with viral infections without COVID-19.

### Retrospective Non-COVID-19 Patient Cohort

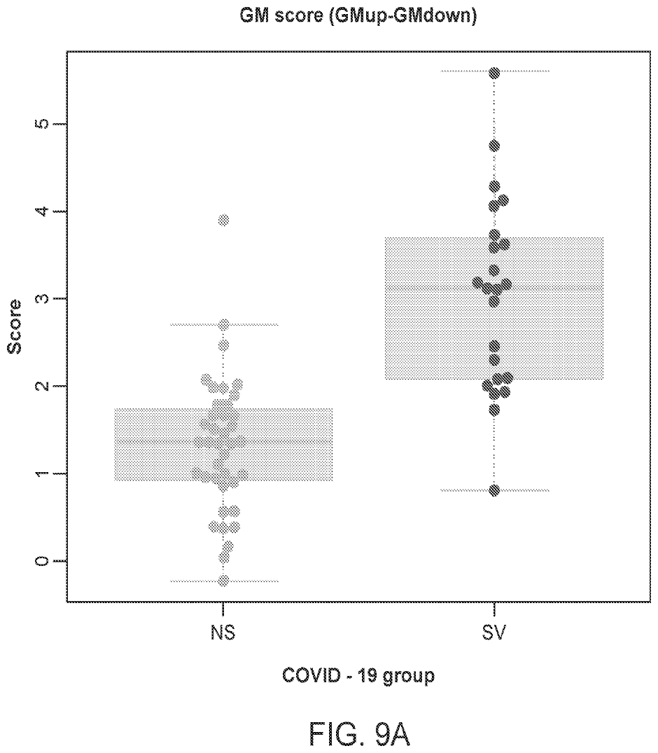

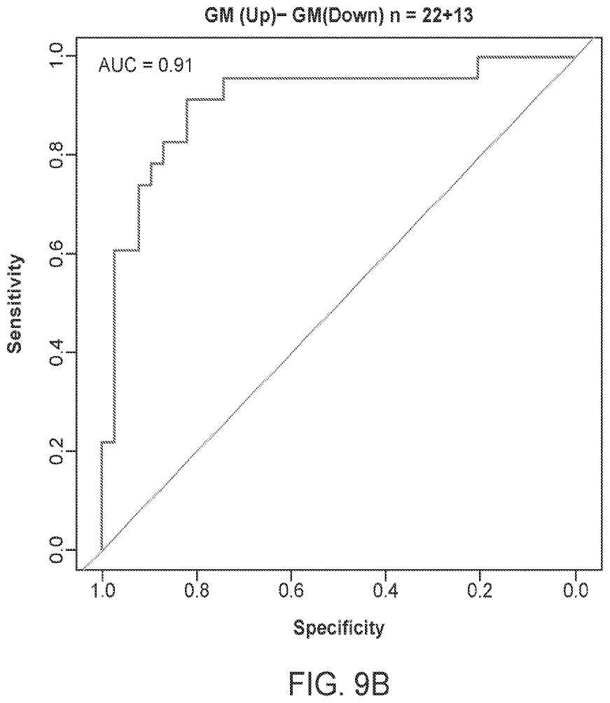

We selected a subset of samples from our previously described database of 34 independent cohorts derived from whole blood or peripheral blood mononuclear cells (PBMCs) (20). From this database we removed all samples that were used in our analysis for identifying the 6-gene signature, leaving 1,417 samples across 21 independent cohorts (Table 11). The samples in these datasets represented the biological and clinical heterogeneity observed in the real-world patient population, including healthy controls and patients infected with 16 different viruses with severity ranging from asymptomatic to fatal viral infection over a broad age range (<12 months to 73 years) (FIG. 9A and Table 11). Notably, the samples were from patients enrolled across 10 different countries representing diverse genetic backgrounds of patients and viruses. Finally, we included technical heterogeneity in our analysis as these datasets were profiled using microarray from different manufacturers.

We renormalized all microarray datasets using standard methods when raw data were available from the GEO database. We applied GC robust multiarray average (gcRMA) to arrays with mismatch probes for Affymetrix arrays. We used normal-exponential background correction followed by quantile normalization for Illumina, Agilent, GE, and other commercial arrays. We did not renormalize custom arrays and used preprocessed data as made publicly available by the study authors. We mapped microarray probes in each dataset to Entrez Gene identifiers (IDs) to facilitate integrated analysis. If a probe matched more than one gene, we expanded the expression data for that probe to add one record for each gene. When multiple probes mapped to the same gene within a dataset, we applied a fixed-effect model. Within a dataset, cohorts assayed with different microarray types were treated as independent.

### Standardized Severity Assignment for Retrospective Non-COVID-19 Patient Samples

We used standardized severity for each of the 1,417 samples as described before (20). Briefly, for each dataset, we used the sample phenotypes as defined in the original publication. We manually assigned a severity category to each sample based on the cohort description for each dataset in the original publication as follows: (1) healthy controls—asymptomatic, uninfected healthy individuals, (2) asymptomatic or convalescents—afebrile asymptomatic individuals who tested positive for a virus or those fully recovered from a viral infection with completely resolved symptoms, (3) mild—symptomatic individuals with viral infection that were either managed as outpatient or discharged from the emergency department (ED), (4) moderate—symptomatic individuals with viral infection who were admitted to the general wards and did not require supplemental oxygen. (5) serious—symptomatic individuals with viral infection who were described as ‘severe’ by original authors, admitted to general wards with supplemental oxygen, or admitted to the intensive care unit (ICU) without requiring mechanical ventilation or inotropic support, (6) critical—symptomatic individuals with viral infection who were on mechanical ventilation in the ICU or were diagnosed with acute respiratory distress syndrome (ARDS), septic shock, or multiorgan dysfunction syndrome (MODS), and (7) fatal—patients with viral infection who died in the ICU.

For datasets that did not provide sample-level severity data (GSE101702, GSE38900, GSE103842, GSE66099, GSE77087), we assigned severity categories as follows. We categorized all samples in a dataset as “moderate” when either (1)>70% of patients were admitted to the general wards as opposed to discharged from the ED, (2)<20% of patients admitted to the general wards required supplemental oxygen, or (3) patients were admitted to the general wards and categorized as ‘mild’ or ‘moderate’ by the original authors. We categorized all samples in a dataset as “severe” when >20% of patients had either (1) been admitted to the general wards and categorized as ‘severe’ by original authors, (2) required supplemental oxygen, or (3) required ICU admission without mechanical ventilation.

### Prospective COVID-19 Patient Cohort

This study was conducted from March-April 2020 at ATTIKON University General Hospital in Athens, Greece (Feb. 26, 2019 approval of the Ethics Committee). Participants were adults with written informed consent provided by themselves or by first-degree relatives in the case of patients unable to consent, with molecular detection of SARS-CoV-2 in respiratory secretions and radiological evidence of lower respiratory tract involvement. PAXgene® Blood RNA tubes were drawn within the first 24 hours from admission along with other standard laboratory parameters. Data collection included demographic information, clinical scores (SOFA, APACHE 11), laboratory results, length of stay and clinical outcomes. Patients were followed up daily for 30 days; severe disease was defined as respiratory failure (PaO2/FiO2 ratio less than 150 requiring mechanical ventilation) or death. PAXgene Blood RNA samples were shipped to Inflammatix, where RNA was extracted and processed using NanoString nCounter®, as previously described (19). The 6-mRNA scores were calculated after locking the classifier weights.

### Healthy Controls

We acquired five whole blood samples from healthy controls through a commercial vendor (BioIVT). The individuals were non-febrile and verbally screened to confirm no signs or symptoms of infection were present within 3 days prior to sample collection. They were also verbally screened to confirm that they were not currently undergoing antibiotic treatment and had not taken antibiotics within 3 days prior to sample collection. Further, all samples were shown to be negative for HIV, West Nile, Hepatitis B, and Hepatitis C by molecular or antibody-based testing. Samples were collected in PAXgene Blood RNA tubes and treated per the manufacturer's protocol. Samples were stored and transported at −80 C.

### Rapid Isothermal Assay

Our goal was to create a rapid assay, and isothermal reactions run much faster than traditional qPCR. Thus, LAMP assays were designed to span exon junctions, and at least three core (FIP/BIP/F3/B3) solutions meeting these design criteria were identified for each marker and evaluated for successful amplification of cDNA and exclusion of gDNA. Where available, loop primers (LF/LB) were subsequently identified for best core solutions to generate a complete primer set. Solutions were down-selected based on efficient amplification of cDNA and RNA, selectivity against gDNA, and the presence of single, homogenous melt peaks. The final primer sets are attached as Table 12.

We designed an analytical validation panel of 61 blood samples from patients in multiple infection classes, including healthy, bacterial or viral. A subset of samples from patients with bacterial or viral infection came from patients with an infection that had progressed to sepsis. Whole blood samples were collected in PAXgene Blood RNA stabilization vacutainers, which preserve the integrity of the host mRNA expression profile at the time of draw. Total RNA was extracted from a 1.5 mL aliquot of each stabilized blood sample using a modified version of the Agencourt RNAdvance Blood kit and protocol. RNA was heat treated at 55° C. for 5 min then snap-cooled prior to quantitation. Total RNA material was distributed evenly across LAMP reactions measuring the five markers in triplicate. LAMP assays were carried out using a modified version of the protocol recommended by Optigene Ltd, and performed on a QuantStudio 6 Real-Time PCR System.

### Statistical Analyses

Analyses were performed in R version 3 and Python version 3.6. The area under the receiver operating characteristic curve (AUROC) was chosen as the primary metric for model evaluation since it provides a general measure of diagnostic test quality without depending on prevalence or having to choose a specific cutoff point.

All validation dataset analyses use the locked 6-mRNA logistic regression output, i.e. predicted probabilities. AUROCs for additional markers (Table 9) are calculated from the available data for each marker. For the logistic regression model that includes the 6-mRNA predicted probabilities along with other markers as predictor variables, conditional multiple imputation was used for values to ensure model convergence. Since AUROC may fail to detect poor calibration on validation data (since subject rankings may still hold), we also demonstrated that a cutoff chosen from training data maintains good sensitivity and specificity in validation data even before recalibration. Due to the relatively small sample size, we made inter-group comparisons without assumptions of normality where possible (Kruskal-Wallis rank sum or Mann-Whitney U test). Medians and interquartile ranges are given for continuous variables.

3. Results

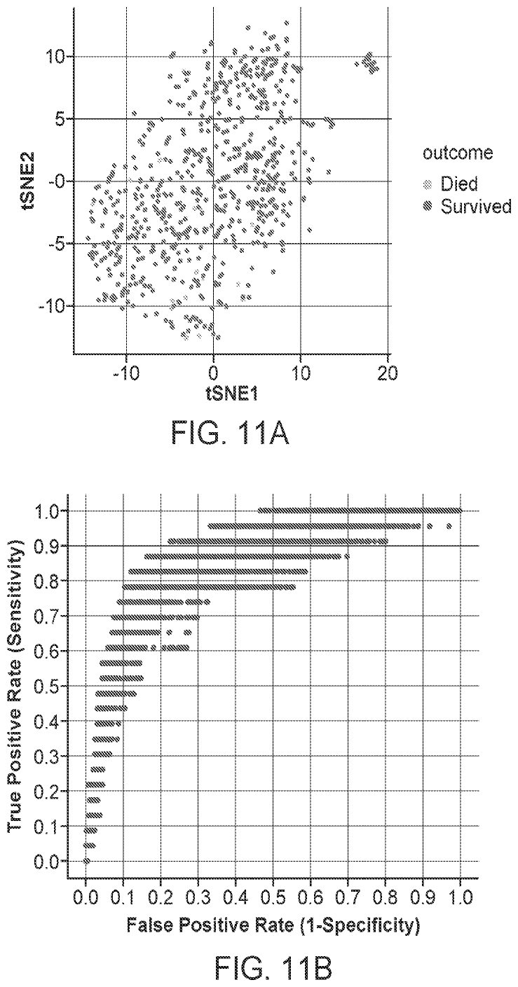

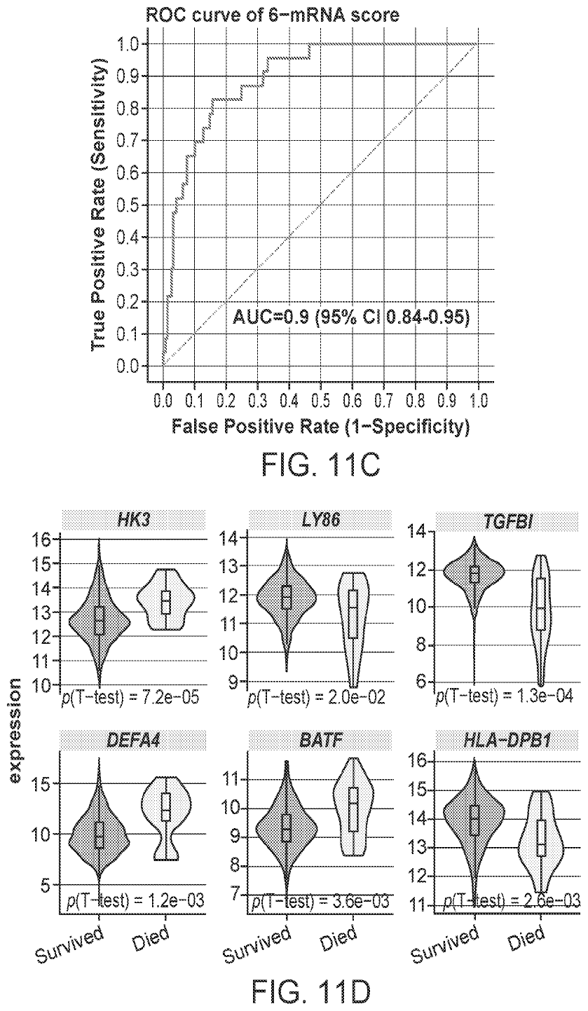

We first identified 21 studies (24-39) with 705 patients with viral infections (none SARS-CoV-2) based on computer-aided adjudication and available outcomes data (see Methods: FIG. 10 and Table 7). These studies included a broad spectrum of clinical, biological, and technical heterogeneity as they profiled blood samples from viral infections from 14 countries using mRNA profiling platforms from four manufacturers (Affymetrix, Agilent, Illumina, Nanostring). Within each dataset, the number of patients who died were very low (two or less for all but one study), meaning traditional approaches for biomarker discovery that rely on a single cohort with sufficient sample size would not have been effective. However, there were sufficient cases (23 deaths within 30 days of sample collection) across these 705 patients. Our previously described approaches for integrating independent datasets and leveraging heterogeneity allowed us to learn across the whole pooled dataset (19, 40, 41). Visualization of the 705 conormalized samples using all genes present across the studies using t-stochastic neighbor embedding (t-SNE), showed that there was no clear separation between the samples from patients who died and those who survived (FIG. 11A).

6-mRNA Logistic Regression-Based Model Accurately Predicts Viral Patient Mortality Across Multiple Retrospective Studies

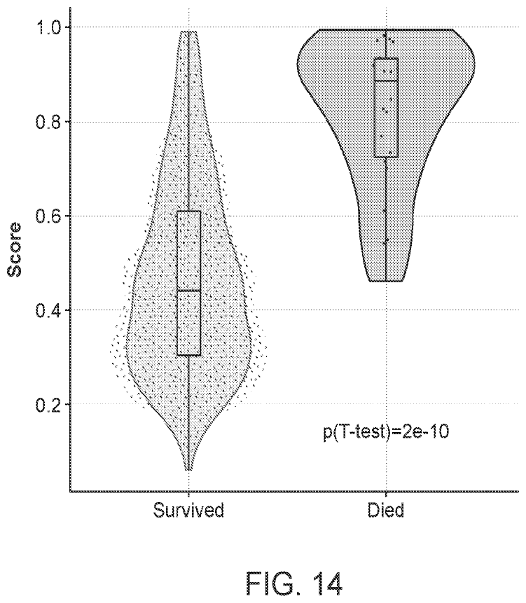

Across the linear machine learning algorithms employed in our analyses, models using logistic regression had the highest mean AUROC for identifying patients with viral infection who died. Further, within logistic regression models, those trained using random cross-validation were more accurate than those trained using other variants of cross-validation. Finally, within the different 6-mRNA logistic regression-based models trained using CV, the model with highest AUROC used the following 6 genes: TGFBI. DEFA4. LY86. BATF. HK3 and HLA-DPB1. It had an AUROC of 0.896 (95% CI: 0.844-0.949) (FIGS. 11B, 11C, and 14). Each of the 6 genes were significantly differentially expressed between patients with viral infections who survived and those who did not, of which 3 genes (DEFA4, BATF. HK3) were higher and 3 genes (TGFBI. LY86, HLA-DPB1) were lower in those who died (FIG. 11D). Based on the cross-validation, the 6-mRNA logistic regression model had a 91% sensitivity and 68% specificity for distinguishing patients with viral infection who died from those who survived. We used this model, referred to as the 6-mRNA classifier, as-is for validation in multiple independent retropective cohorts and a prospective cohort.

6-mRNA Classifier is an Age-Independent Predictor of Mortality in Patients with Viral Infections

Age is a known significant predictor of 30-day mortality in patients with respiratory viral infections. To assess the added value of the new prognostic information of the 6-mRNA classifier with regards to age in the training data, we fit a binary logistic regression model with age and pooled cross-validation 6-mRNA classifier probabilities as independent variables. The 6-mRNA score was significantly associated with increased risk of 30-day mortality (P<0.001), but age was not (P=0.06).

Validation of the 6-mRNA Classifier in Multiple Independent Retrospective Cohorts

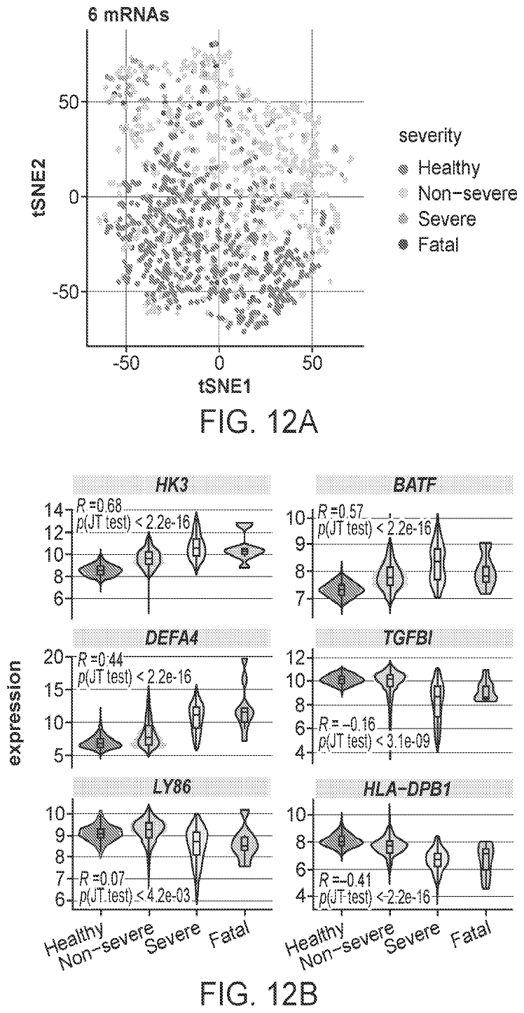

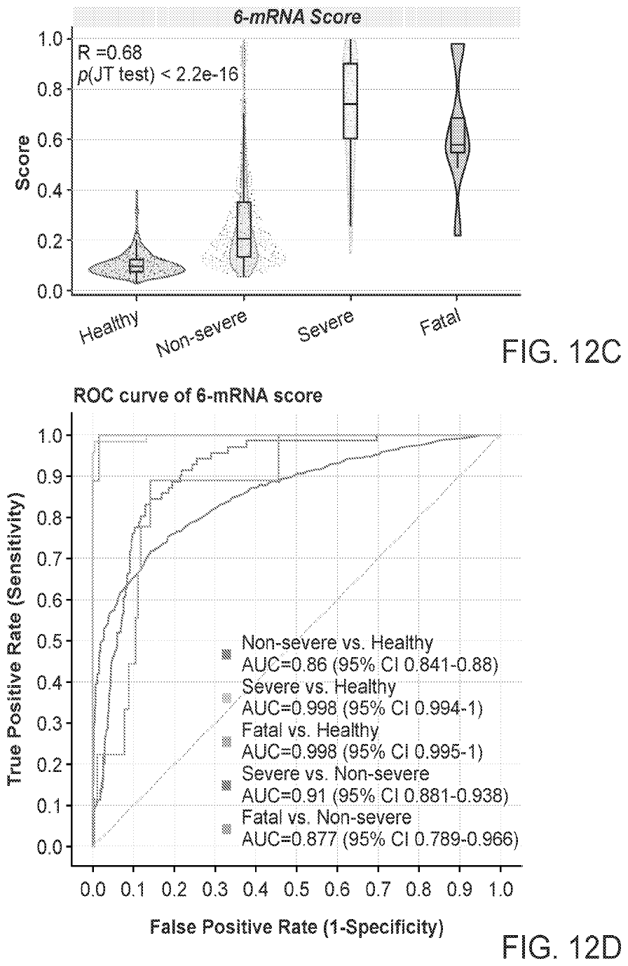

We applied the locked 6-mRNA classifier to 1,417 transcriptome profiles of blood samples across 21 independent cohorts from patients with viral infections (663 healthy controls, 674 non-severe, 71 severe, 7 fatal) in 10 countries (Table 11). Visualization of the 1,417 samples using expression of the 6 genes showed patients with severe outcome clustered closer (FIG. 12A). Among the 6 genes, over-expressed genes (HK3, DEFA4, BATF) were positively correlated with severity of viral infection, and under-expressed gene (HLA-DPB1, LY86, 7UFB1) were negatively correlated with severity (FIG. 12B). Importantly, the 6-mRNA classifier score was positively correlated with severity and was significantly higher in patients with severe or fatal viral infection than those with non-severe viral infections or healthy controls (FIG. 12C). Finally, the 6-mRNA classifier score distinguished patients with severe viral infection from those with non-severe viral infection (AUROC=0.91, 95% CI: 0.881-0.938) and healthy controls (AUROC=0.998, 95% CI: 0.994-1) (FIG. 12D).

We plotted ROC curves to assess the discriminative ability of the 6-mRNA classifier among the following subgroups of clinical interest: healthy controls, non-severe cases, severe, and fatal outcomes (FIG. 12D). Healthy controls are presented (though not mixed with non-severe viral infections in comparison) since some viral infections such as COVID-19 can be asymptomatic. All pairwise comparisons showed robust performance of the classifier on the independent data, achieving AUROC point-estimates between 0.86 (non-severe vs. healthy) and 1 (severe vs. healthy).

Prospective Validation of the 6-mRNA Logistic Regression Model in an Independent Cohort

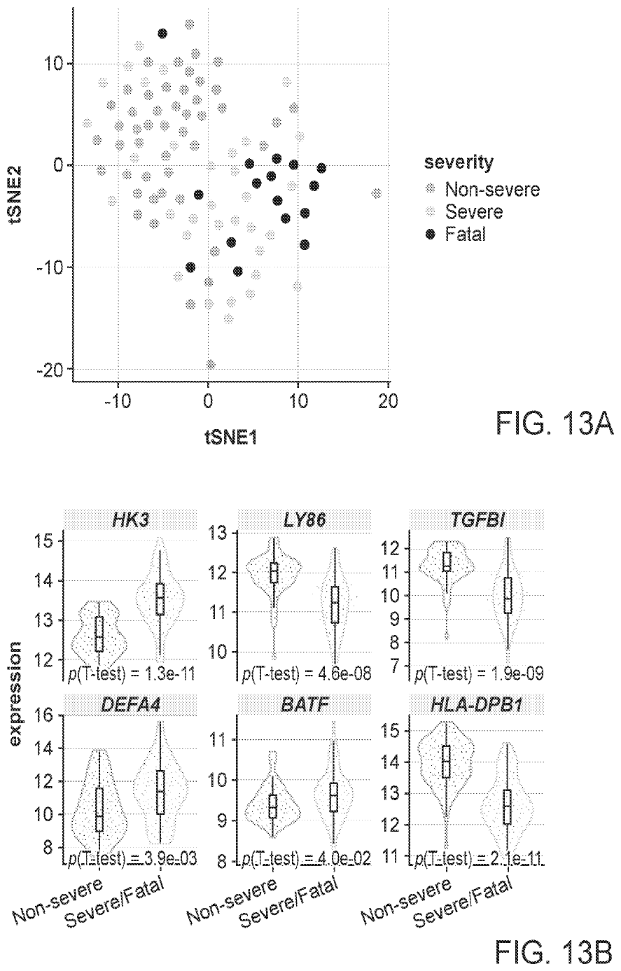

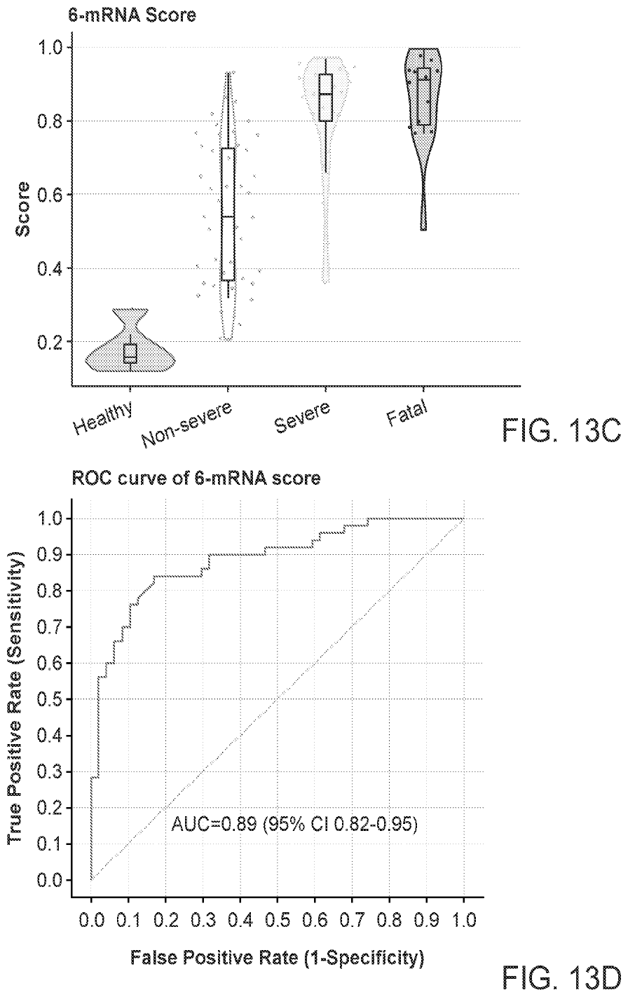

We prospectively enrolled 97 adult patients with pneumonia by SARS-CoV-2 in Athens, Greece. There were 47 patients with non-severe COVID-19 disease, whereas 50 had severe COVID-19, of which 16 died (Table 8). Interestingly, visualization of these samples in low dimension using expression of the 6 mRNAs (without the classifier) did not distinguish patients with severe COVID-19 disease from those with non-severe disease (FIG. 13A). When comparing expression of the 6 mRNAs in patients with non-severe COVID-19 disease to those with severe disease, expression of each changed statistically significant in the same direction as the training data (P<0.05) (FIG. 13B).

We applied the locked 6-mRNA classifier to the 97 COVID-19 patients and the 5 healthy controls. Strikingly, the classifier distinguished among healthy controls, patients with non-severe COVID-19, and patients with severe COVID-19 and mortality (FIG. 13C). In particular, the model distinguished patients with severe respiratory failure from non-severe patients with an AUROC of 0.89 (95% CI: 0.82-0.95; FIG. 13D).

We also assessed whether the 6-mRNA score is an independent predictor of severity in patients with COVID-19 by including other predictors of seventy (age, SOFA score, CRP, PCT, lactate, and gender) in a logistic regression model. As expected, due to small sample size, and correlations between markers, no markers except SOFA were statistically significant predictors of severe respiratory failure (Table 13).

For clinical applications, AUROC is a more relevant indicator of marker performance. To that end, we compared the 6-mRNA score to other clinical parameters of severity using AUROC (Table 9). The 6-mRNA score was the most accurate predictor of severe respiratory failure and death except SOFA. The AUROC confidence intervals were overlapping because the study was not powered to detect statistically significant differences. As a proxy for assessing how the 6-mRNA score might add to a clinician's bedside severity assessment, we evaluated whether a combination of our classifier with the SOFA score improves over SOFA alone for the prediction of severe respiratory failure. The two scores together had an AUROC of 0.95; the continuous net reclassification improvement (cNRI) was 0.43 [95% CI: 0.04-0.81, P=0.03]. Together, these results suggest a potential improvement in clinical risk prediction when adding the 6-mRNA score to standard risk predictors, but definitive conclusion requires validation in additional independent data.

### Translation to a Clinical Report

To improve utility and adoption, a risk prediction score should be presented to clinicians in an intuitive and actionable test report. To that end, we discretized the 6-mRNA score in three bands: low-risk, intermediate-risk, and high-risk of severe outcome. The performance characteristics of each band are shown in Table 10. The table shows performance of the test on retrospective data (excluding healthy controls) using two versions of decision thresholds: thresholds optimized on the training data (Table 10A), and thresholds optimized using the retrospective test set (Table 10B). The outcome was severe infection. Tables 10C, 10D show corresponding results on the COVID-19 data, using severe respiratory failure as outcome.

### Translation to a Rapid Assay

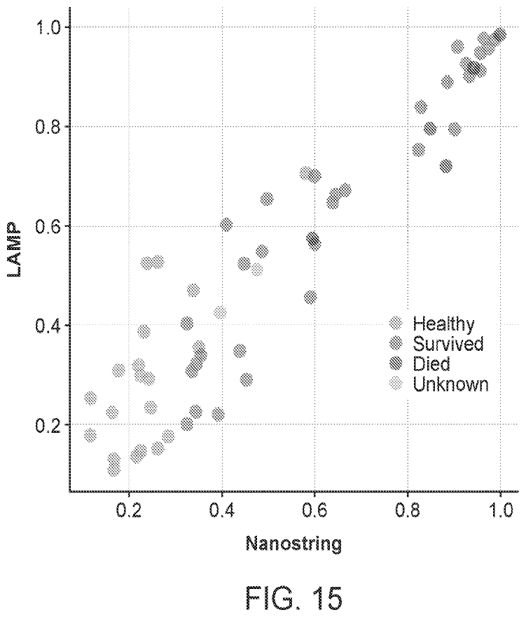

Any risk prediction score should be rapid enough to fit into clinical workflows. We thus developed a LAMP assay as a proof of concept for a rapid 6-mRNA test. We further showed that across 61 clinical samples from healthy controls and acute infections of varying severities that the LAMP 6-mRNA score and the reference NanoString 6-mRNA score had very high correlation (r=0.95; FIG. 15). These results demonstrate that with further optimization the 6-mRNA model could be translated into a clinical assay to run in less than 30 minutes.

4. Discussion

The severe economic and societal cost of the ongoing COVID-19 pandemic, the fourth viral pandemic since 2009, has underscored the urgent need for a prognostic test that can help stratify patients as to who can safely convalesce at home in isolation and who needs to be monitored closely. Here we integrated 705 peripheral blood transcriptome profiles across 21 heterogeneous studies from patients with viral infections, none of whom were infected with SARS-CoV-2. Despite the substantial biological, clinical, and technical heterogeneity across these studies, we identified a 6-mRNA host-response signature that distinguished patients with severe viral infections from those without. We demonstrated generalizability of this 6-mRNA model first in a set of 21 independent heterogeneous cohorts of 1,417 retrospectively profiled samples, and then in an independent prospectively collected cohort of patients with SARS-CoV-2 infection in Greece. In each validation analysis, the 6-mRNA classifier accurately distinguished patients with severe outcome from those with non-severe outcomes, irrespective of the infecting virus, including SAR-CoV-2. Importantly, across each analysis, the 6-mRNA classifier had similar accuracy, measured by AUROC, demonstrating its generalizability and robustness to biological, clinical, and technical heterogeneity. Although this study was focused on development of a clinical tool, not a description of transcriptome-wide changes, the applicability of the signature across viral infections further demonstrates that host factors associated with severe outcomes are conserved across viral infections, which is in line with our recent large-scale analysis (20).

While many risk-stratification scores and biomarkers exist, few are focused specifically on viral infections. Of the recent models specifically designed for COVID-19, most are trained and validated in the same homogenous cohorts, and their generalizability to other viruses is unknown because they have not been tested across other viral infections (14). Consequently, when a new virus, such as SARS-CoV-2, emerges, their utility is substantially limited. However, we have repeatedly demonstrated that the host response to viral infections is conserved and distinct from the host response to other acute conditions (15-20).

Here, building upon our prior results, we developed a 6-mRNA classifier specifically trained in patients with viral infection to risk stratify better than other existing biomarkers. Further, the only assay authorized for clinical use in risk-stratifying COVID-19 (IL-6 measured in blood), substantially underperformed our proposed 6-mRNA model here. That said, the nominal improvement over existing biomarkers (Table 9) for prediction of severe respiratory failure requires larger cohorts to confirm statistical significance. The 6-mRNA score is nominally worse than SOFA, but SOFA requires 24 hours to calculate, while the 6-mRNA score could be run in 30 minutes, demonstrating its utility as a triage test. The synergy (positive NRI) in combination with SOFA also suggests that the 6-mRNA score could improve practice in combination with clinical gestalt. The 6-mRNA score has been reduced to practice as a rapid isothermal quantitative RT-LAMP assay, suggesting that it may be practical to implement in the clinic with further development.

Our goal in this study was not to investigate underlying biological mechanisms, but to address the urgent need for a prognostic test in SARS-CoV-2 pandemic, and to improve our preparedness for future pandemics. However, using immunoStates database (metasignature.khatrilab.stanford.edu) (42), we found 5 out of the 6 genes (HK3. DEFA4, TGFBI. LY86. HLA-DPB1) are highly expressed in myeloid cells, including monocytes, myeloid dendritic cells, and granulocytes. This is in line with our recent results demonstrating that myeloid cells are the primary source of conserved host response to viral infection (20). Further, we have previously found that DEFA4 is over-expressed in patients with dengue virus infection who progress to severe infection (43), and in those with higher risk of mortality in patients with sepsis (18). HLA-DPB1 belongs to the HLA class 11 beta chain paralogues, and plays a central role in the immune system by presenting peptides derived from extracellular proteins. Class II molecules are expressed in antigen presenting cells (B lymphocytes, dendritic cells, macrophages). Reduced expression of HLA-DPB1 in patients with severe outcome suggests dysfunctional antigen presentation that should be further investigated. Similarly. BATF is significantly over-expressed, and TGFBI is significantly under-expressed in patients with sepsis compared to those with systemic inflammatory response syndrome (SIRS) (15). Finally, lower expression of TGFBI and LY86 in peripheral blood is associated with increased risk of mortality in patients with sepsis (18). These results further suggest that there may be a common underlying host immune response associated with severe outcome in infections, irrespective of bacterial or viral infection. Consistent differential expression of these genes in patients with a severe infectious disease across heterogeneous datasets lend further support to our hypothesis that dysregulation in host response can be leveraged to stratify patients in high- and low-risk groups.

Our study has several limitations. First, our study uses retrospective data with large amount of heterogeneity for discovery of the 6-mRNA signature: such heterogeneity could hide unknown confounders in classifier development. However, our successful representation of biological, clinical, and technical heterogeneity also increased the a priori odds of identifying a parsimonious set of generalizable prognostic biomarkers suitable for clinical translation as a point-of-care. Second, owing to practical considerations for urgent need, we focused on a preselected panel of mRNAs. It is possible that similar analysis using the whole transcriptome data would find additional signatures, though with less clinical data. Third, we only considered linear models. It is possible that more complex models that account for non-linear relationships may be more accurate, but also may be overfit. Fourth, a common limitation in all these types of pandemic observational studies is a lack of understanding of the effect of time from symptoms onset. Finally, additional larger prospective cohorts are needed to further confirm the accuracy of the 6-mRNA model in distinguishing patients at high risk of progressing to severe outcomes from those who do not.

Overall, our results show that once translated into a rapid assay and validated in larger prospective cohorts, this 6-mRNA prognostic score could be used as a clinical tool to help triage patients after diagnosis with SARS-CoV-2 or other viral infections such as influenza. Improved triage could reduce morbidity and mortality while allocating resources more effectively. By identifying patients at high risk to develop severe viral infection, i.e., the group of patients with viral infection who will benefit the most from close observation and antiviral therapy, our 6-mRNA signature can also guide patient selection and possibly endpoint measurements in clinical trials aimed at evaluating emerging anti-viral therapies. This is particularly important in the setting of current COVID-19 pandemic, but also useful in future pandemics or even seasonal influenza.

TABLE 7
Characteristics of viral infection studies used for training.

| | | | | N | | | | |
|---|---|---|---|---|---|---|---|---|
| | First | | Timing of | (survivors/ | Age | | | |
| Study | author | | sample | non- | (Median, | Male | | |
| identifier | or PI | Study description | collection | survivors) | IQR) | (n (%)) | Country | Platform |
| E-MEXP- | Almansa | Patients hospitalized | Hospital/ | 5 (5/0) | Unk. | 5(100) | Spain | Agilent |
| 3589 | | with COPD* | ICU | | | | | |
| | | exacerbation | admission | | | | | |
| E-MTAB- | Almansa | Surgical patients with | Average | 3 (3/0) | 78.0 (71.5- | 3(100) | Spain | Agilent |
| 1548 | | sepsis (EXPRESS) | post- | | 79.5) | | | |
| | | | operation | | | | | |
| | | | day 4 | | | | | |
| E-MEXP- | Van de | Uncomplicated dengue | Within 48 h | 21 (21/0) | Unk. | Unk. | Indonesia | Affymetrix |
| 3162 | Weg | | of onset | | | | | |
| GSE 13015 | Pankla | Sepsis, many cases | Within 48 h | 3 (2/1) | 54.0 (46.0- | 1(33) | Thailand | Illumina |
| (GPL6102) | | from burkholderia | of diagnosis | 2 (2/0) | 55.5) | | | |
| GSE 13015 | | | | | 64.5 (56.2- | 1(50) | | Illumina |
| (GPL6947) | | | | | 72.8) | | | |
| GSE21802 | Bermejo- | Pandemic H1N1 in | Within 48 h | 6 (5/1) | Unk. | Unk. | Canada | Illumina |
| | Martin | ICU** | of ICU | | | | | |
| | | | admission | | | | | |
| GSE22098 | Berry | Patients with active | At | 39 (39/0) | 31.0 (19.0- | 6(15) | UK. | Illumina |
| | | TB*** and other | admission | | 47.0) | | South | |
| | | inflammatory and | | | | | Africa | |
| | | infectious diseases | | | | | | |
| GSE27131 | Berdal | Severe H1N1 influenza | Admission | 3 (2/1) | 38.0 (31.5- | 3(100) | Norway | Affymetrix |
| | | | to ICU | | 46.0 | | | |
| GSE28991 | Naim | Acute dengue fever | Within 72h | 11 (11/0) | Unk. | Unk. | Singapore | Illumina |
| | | | of onset | | | | | |
| GSE32707 | Dolinay | Critically ill patients in | Admission | 7 (5/2) | 45.0 (39.0- | 4(57) | USA | Illumina |
| | | ICU (Sepsis, SIRS | to ICU | | 50.5) | | | |
| | | and/or ARDS) | | | | | | |
| GSE40012 | Parnell | Bacterial or influenza A | Admission | 11 (11/0) | Unk. | 4(36) | Australia | Illumina |
| | | pneumonia or SIRS | to ICU | | | | | |
| GSE54514 | Parnell | Sepsis patients in ICU | Admission | 2 (2/0) | 62.5 (60.2- | 1(50) | Australia | Illumina |
| | | | to ICU | | 64.8) | | | |
| GSE51808 | Kwissa | Acute dengue fever | 1-8 days | 28 (28/0) | | Unk. | Thailand | Affymetrix |
| | | | after onset | | | | | |
| GSE60244 | Suarez | Lower respiratory tract | Within 24 h | 62 (62/0) | 59.0 (50.0- | 24(39) | USA | Illumina |
| | | infections | of | | 74.5) | | | |
| | | | admission | | | | | |
| GSE65682 | Scicluna | Suspected but negative | Within 24 h | 9 (7/2) | 67.0 (63.0- | 7(78) | Netherlands | Affymetrix |
| | | for CAP**** | of ICU | | 73.0) | | | |
| | | | admission | | | | | |
| GSE68310 | Zhai | Outpatients with acute | Within 48 h | 75 (75/0) | 21.0 (20.4- | 34(45) | USA | Illumina |
| | | respiratory viral | of onset | | 22.3) | | | |
| | | infections | | | | | | |
| GSE82050 | Tang | Moderate and severe | Within 24 h | 17 (17/0) | 55.0 (45.0- | Unk. | Germany | Agilent |
| | | influenza infection | of | | 72.0) | | | |
| | | | admission | | | | | |
| GSE95233 | Venet | Septic shock patients in | Admission | 7 (5/2) | 47.0 (42.0- | 5(71) | France | Affymetrix |
| | | ICU | to ICU | | 65.0) | | | |
| Australia/ | Tang | Community or hospital | At | 332 | 48.0 (32.0- | 129(39) | Australia | Nanostring |
| WIMR | | clinics with influenza- | presentation | (321/11) | 63.5) | | | |
| | | like illness | | | | | | |
| Stanford ICU | Rogers | Suspected sepsis with | Admission | 8 (6/2) | 62.0 (55.5- | 4(50) | USA | Nanostring |
| databank | | ARDS risk factors | to ICU | | 67.2) | | | |
| PROMPT | Giamarel | Suspected infection | Admission | 1 (1/0) | 78.0 | 0(0) | Greece | Nanostring |
| | los- | with 2+ SIRS | to ED | | | | | |
| | Bourboulis | | | | | | | |
| PREVISE | Herrero | Outpatient urgent care | At | 53 (52/1) | 78.0 (66.0- | 33(62) | Spain | Nanostring |
| | | with suspected CAP | presentation | | 87.0) | | | |

*COPD, chronic pulmonary obstruction disorder;
**ICU, intensive care unit;
***TB, tuberculosis;
****CAP, community-acquired penumonia

TABLE 8
Demographics, severity scores, and severity markers for the prospective COVID-19 cohort, overall and split by mortality.
P-values correspond to Mann-Whitney tests for difference of means and chi-square tests for difference of proportions
between the survival and mortality groups. Unless indicated otherwise, numbers shown are median [IQR].

| Variable | Overall | | Death | | Survival | | P value |
|---|---|---|---|---|---|---|---|
| N | 97 | | 16 | | 81 | | |
| Age years | 62 | [52, 72.25] | 68.50 | [62.75, 84.25] | 60.00 | [50.75, 70.25] | 0.003 |
| Gender = Male (%) | 68 | (70.1) | 12 | (75.0) | 56 | (69.1) | 0.865 |
| White blood cells/mm3 | 6770 | [5145, 10227.50] | 8540.00 | [5542.50, 12510.00] | 6480.00 | [5145.00, 9622.50] | 0.275 |
| Neutrophils (%) | 78.10 | [68.35, 86.60] | 88.95 | [86.40, 93.03] | 77.09 | [65.22, 83.75] | <0.001 |
| Lymphocytes (%) | 12.70 | [7.20, 21.15] | 6.70 | [3.65, 9.65] | 14.03 | [9.00, 22.42] | <0.001 |
| Platelets/mm3 | 215000 | [172900, 266000] | 249050 | [180750, 298000] | 214000 | [172600, 260800] | 0.176 |
| D-dimer ng/ml | 977.90 | [476.25, 2560.00] | 4480.00 | [2440.00, 13161.50] | 850.00 | [437.50, 1947.50] | <0.001 |
| CRP mg/l | 107.00 | [31.60, 222.50] | 224.75 | [142.89, 260.75] | 79.10 | [28.80, 202.00] | 0.002 |
| SOFA score | 3.00 | [1, 00, 6, 00] | 5.50 | [4.00, 6, 25] | 2 | [1, 6] | 0.006 |
| APACHE II | 7.00 | [5.00, 11.00] | 11.00 | [8.00, 13.50] | 7 | [4, 9] | 0.001 |
| Length of hospital stay | 13.00 | [11.00, 20.00] | 13 | [8.75, 17.25] | 13 | [11, 20] | 0.410 |
| Severe respiratory failure (%) | 50 | (51.5) | 16 | (100.0) | 34 | (42.0) | <0.001 |

Table 9. Prognostic power of the 6-mRNA signature classifier and comparator scores and markers in the independent COVID-19 cohort. Shown are AUROCs for non-missing data, plus 95% Cf. The final column is a ‘fair’ assessment of the 6-mRNA signature classifier, i.e. the performance on the subset of patients that was available to the comparator.

TABLE 9A
Prognostic power for predicting severe respiratory failure.
Bold font indicates predictor with higher AUROC, which
in nearly all cases is the 6-mRNA classifier.

| Comparator | Number | Comparator | 6-mRNA classifier |
|---|---|---|---|
| Marker | Available | AUROC | AUROC |
| 6-mRNA classifier | 97 | | 0.89 (0.82-0.95) |
| SOFA | 96 | 0.93 (0.87-0.98) | 0.89 (0.82-0.95) |
| APACHE II | 93 | 0.83 (0.75-0.91) | 0.89 (0.83-0.96) |
| Age | 96 | 0.78 (0.69-0.87) | 0.89 (0.82-0.95) |
| PCT | 76 | 0.80 (0.70-0.90) | 0.89 (0.81-0.96) |
| CRP | 97 | 0.86 (0.79-0.94) | 0.89 (0.82-0.95) |
| Lactate | 45 | 0.75 (0.61-0.90) | 0.82 (0.69-0.94) |
| IL-6 | 97 | 0.73 (0.63-0.83) | 0.89 (0.82-0.95) |
| suPAR | 97 | 0.79 (0.70-0.88) | 0.89 (0.82-0.95) |

TABLE 9B
Prognostic power for predicting mortality. Bold
font indicates predictor with the higher AUROC.

| Comparator | Number | Comparator | 6-mRNA classifier |
|---|---|---|---|
| Marker | Available | AUROC | AUROC |
| 6-mRNA classifier | 97 | | 0.78 (0.64-0.92) |
| SOFA | 96 | 0.72 (0.57-0.87) | 0.78 (0.64-0.92) |
| APACHE II | 93 | 0.76 (0.61-0.90) | 0.77 (0.63-0.91) |
| Age | 96 | 0.74 (0.59-0.89) | 0.78 (0.64-0.92) |
| PCT | 76 | 0.73 (0.56-0.89) | 0.77 (0.61-0.93) |
| CRP | 97 | 0.74 (0.59-0.89) | 0.78 (0.64-0.92) |
| Lactate | 45 | 0.78 (0.60-0.95) | 0.80 (0.63-0.97) |
| IL-6 | 97 | 0.57 (0.41-0.73) | 0.78 (0.64-0.92) |
| suPAR | 97 | 0.74 (0.60-0.89) | 0.78 (0.64-0.92) |

Table 10. Test characteristics of the 6-mRNA score in non-COVID-19 and COVID-19 patients using the three-band test report. “Severe in band” is the number of patients with severe viral infection assigned to the corresponding band. “Non-severe in band” is the number of patients with non-severe viral infection assigned to the corresponding band. The “Percent severe in band” is the percentage of patients in the band who had severe outcome. The “In-band” column is the percentage of patients assigned by the classifier to the corresponding band in the retrospective study.

TABLE 10A
non-COVID-19 results. The band thresholds were set using training data and
locked.

| | | | Percent | | | | |
|---|---|---|---|---|---|---|---|
| | Severe in | Non-severe | severe | | | Likelihood | |
| Band | band | in band | in band | Sensitivity | Specificity | ratio | In-band |
| Low risk | 2 | 419 | 0.5% | 98% | 62% | 0.04 | 56% |
| Intermediate risk | 68 | 247 | 22% | 85% | 63% | 2.3 | 42% |
| High risk | 10 | 8 | 56% | 12% | 99% | 11 | 2.4% |

TABLE 10B
non-COVID-19 results. The band thresholds were set using the retrospective
data.

| | | | Percent | | | | |
|---|---|---|---|---|---|---|---|
| | Severe in | Non-severe | severe | | | Likelihood | |
| Band | band | in band | in band | Sensitivity | Specificity | ratio | In-band |
| Low risk | 9 | 540 | 1.6% | 89% | 80% | 0.14 | 73% |
| Intermediate risk | 2 | 19 | 9.5% | 2.5% | 97% | 0.89 | 2.8% |
| High risk | 69 | 115 | 38% | 86% | 83% | 5.1 | 24% |

TABLE 10C
COVID-19 results. The band thresholds were set using training data and locked.

| | | | Percent | | | | |
|---|---|---|---|---|---|---|---|
| | Severe in | Non-severe | severe | | | Likelihood | |
| Band | band | in band | in band | Sensitivity | Specificity | ratio | In-band |
| Low risk | 4 | 25 | 14% | 92% | 53% | 0.15 | 30% |
| Intermediate risk | 3 | 7 | 30% | 6% | 85% | 0.4 | 10% |
| High risk | 43 | 15 | 74% | 86% | 68% | 2.7 | 60% |

TABLE 10D
COVID-19 results. The band thresholds were set using the prospective data.

| | | | Percent | | | | |
|---|---|---|---|---|---|---|---|
| | Severe in | Non-severe | severe | | | Likelihood | |
| Band | band | in band | in band | Sensitivity | Specificity | ratio | In-band |
| Low risk | 5 | 32 | 14% | 90% | 68% | 0.15 | 38% |
| Intermediate risk | 5 | 8 | 38% | 10% | 83% | 0.59 | 13% |
| High risk | 40 | 7 | 85% | 80% | 85% | 5.4 | 48% |

TABLE 11
Characteristics of retrospective viral infection (non-COVID-19) studies used for
independent validation.

| | | | | N | | | | |
|---|---|---|---|---|---|---|---|---|
| | First | | Timing of | (total/healthy/ | | | | |
| | author or | | sample | non-severe/ | | Male | | |
| Study identifier | PI | Study description | collection | severe/fatal) | Age | (n (%)) | Country | Platform |
| GSE103842 | Rodriguez- | RSV infected | Within 24 hours | 74, 12, 62, 0, 0 | Child | 48(65) | USA | Illumina |
| | Fernandez | infants | of | | (0-2 | | | |
| | | | hospitalization | | years) | | | |
| GSE111368 | Dunning | Patients with | Samples were | 239, 130, 81, | Adult | 111(46) | UK | Illumina |
| | | severe influenza | obtained at three | 28, 0 | (18-71 | | | |
| | | with or without | time points: T1 | | years) | | | |
| | | bacterial co- | (recruitment), T2 | | | | | |
| | | infection | (approximately | | | | | |
| | | | 48 h after T1) | | | | | |
| | | | and T3 (at least 4 | | | | | |
| | | | weeks after T1) | | | | | |
| GSE20346 | Parnell | Adults with CAP | Hospital | 22, 18, 0, 4, 0 | Adult | 7(32) | Australia | Illumina |
| | | | admission | | (21-75 | | | |
| | | | | | years) | | | |
| GSE27131 | Berdal | Patients with | Admission to | 13, 7, 0, 3, 3 | Adult | 9(69) | Norway | Affymetrix |
| | | documented | ICU | | (25-59 | | | |
| | | influenza, bilateral | | | years) | | | |
| | | chest infiltrates, | | | | | | |
| | | and in need of | | | | | | |
| | | ventilation | | | | | | |
| | | support, without | | | | | | |
| | | significant co- | | | | | | |
| | | morbidity | | | | | | |
| GSE77087 | de | Outpatient and | Either at the | 104, 23, 81, 0, 0 | Child | 67(64) | USA | Illumina |
| | Steenhuijsen | inpatient RSV | outpatient clinics | | (0-2 | | | |
| | | patients | or within a | | years) | | | |
| | | | median of 24 | | | | | |
| | | | hours of | | | | | |
| | | | admission in the | | | | | |
| | | | pediatric ward or | | | | | |
| | | | the pediatric ICU | | | | | |
| GSE67059 | Heinonen | Asymptomatic | ED (outpatients) | 137, 37, 100, 0, 0 | Child | 87(64) | USA, | Illumina |
| | | and symptomatic | or within 48 | | (0-2 | | Finland, | |
| | | rhinovirus in | hours of | | years) | | Spain | |
| | | children | hospitalization | | | | | |
| | | | (inpatients) | | | | | |
| GSE21802 | Bermejo- | Patients attending | Admission to | 20, 4, 12, 2, 2 | Adult | 12(60) | Spain | Illumina |
| | Martin | to the participants | ICU | | (18-65 | | | |
| | | ICUs with | | | years) | | | |
| | | primary viral | | | | | | |
| | | pneumonia during | | | | | | |
| | | the acute phase of | | | | | | |
| | | influenza virus | | | | | | |
| | | illness with acute | | | | | | |
| | | respiratory | | | | | | |
| | | distress and | | | | | | |
| | | unequivocal | | | | | | |
| | | alveolar | | | | | | |
| | | opacification | | | | | | |
| | | involving two or | | | | | | |
| | | more lobes with | | | | | | |
| | | negative | | | | | | |
| | | respiratory and | | | | | | |
| | | blood bacterial | | | | | | |
| | | cultures at | | | | | | |
| | | admission | | | | | | |
| GSE66099 | Sweeney, | Septic children in | Admission to | 58, 47, 0, 9, 2 | Child | 32(55) | USA | Affymetrix |
| | Alder | PICU | ICU | | (0-10 | | | |
| | | | | | years) | | | |
| GSE101702 | Tang. | Influenza patients | Within 24 hours | 159, 52, 107, 0, 0 | Adult | 63(40) | Australia, | Agilent |
| | Zerbib | with varying | of their | | (17-90 | | Canada, | |
| | | severity of | presentation to | | years) | | Germany | |
| | | infection | the hospital | | | | | |
| GSE17156_FLU | Zaas | Influenza | Multiple time | 25, 17, 8, 0, 0 | Adult | 12(48) | USA, | Affymetrix |
| | | challenge study | points | | (>18 | | UK | |
| | | | | | years) | | | |
| GSE17156_RSV | Zaas | RSV challenge | Multiple time | 29, 20, 9, 0, 0 | Adult | 16(55) | USA, | Affymetrix |
| | | study | points | | (>18 | | UK | |
| | | | | | years) | | | |
| GSE17156_RHINO | Zaas | Rhinovirus | Multiple time | 29, 19, 10, 0, 0 | Adult | 16(55) | USA, | Affymetrix |
| | | challenge study | points | | (>18 | | UK | |
| | | | | | years) | | | |
| GSE40012 | Parnell | Adults with CAP | Within 24 hours | 38, 36, 0, 2, 0 | Adult | 13(34) | Australia, | Illumina |
| | | | of admission to | | (22-75 | | Hong | |
| | | | ICU | | years) | | Kong | |
| GSE68004 | Jaggi | Kawasaki disease | Hospital | 56, 37, 19, 0, 0 | Child | 25(45) | USA | Illumina |
| | | compared to other | admission | | (0-16 | | | |
| | | febrile patients | | | years) | | | |
| EMTAB5195 | Jong | Respiratory | Within 24 hours | 43, 4, 21, 18, 0 | Child | 27(63) | Netherlands | Affymetrix |
| | | syncytial virus | of presentation to | | (0-2 | | | |
| | | infected infants | the hospital | | years) | | | |
| GSE6269 | Ramilo | Sepsis patients | | 24, 6, 18, 0, 0 | Child | 15(62) | USA | Affymetrix, |
| | | with influenza or | | | (0-18 | | | Illumina |
| | | bacterial infection | | | years) | | | |
| GSE68310 | Zhai | Influenza and | Multiple time | 157, 128, 29, 0, 0 | Adult | 77(49) | USA | Illumina |
| | | other acute | points | | (18-49) | | | |
| | | respiratory viral | | | | | | |
| | | infections | | | | | | |
| GSE117827 | Yu | Children with | Hospital | 19, 6, 13, 0, 0 | Child | 14(74) | USA | Affymetrix |
| | | acute viral | admission | | (0-11 | | | |
| | | infection | | | years) | | | |
| GSE25504 | Smith, | Septic neonates | Hospital | 9, 6, 3, 0, 0 | Child | 9(100) | UK | Affymetrix |
| | Dickinson | | admission, at the | | (0-1 | | | |
| | | | time of first | | year) | | | |
| | | | clinical signs of | | | | | |
| | | | suspected sepsis | | | | | |
| GSE4607 | Wong, | Septic children in | Within 24 hours | 22, 15, 0, 5, 2 | Child | 14(64) | USA | Affymetrix |
| | Cvijanovich | PICU | of admission to | | (0-10 | | | |
| | | | ICU | | years) | | | |
| GSE38900 | Mejias | Children with | Hospital | 140, 39, 101, 0, 0 | Child | 76(54) | USA, | Illumina |
| | | acute LRTI | admission | | (0-2 | | Finland | |
| | | | | | years) | | | |

TABLE 12
Oligonucleotide sequences for detection of 6 informative viral severity markers.

| Oligo ID | Sequence |
|---|---|
| PD HK3v4 F3 | ACCTGAGGAGAGTGACTAGCTTCT |
| PD HK3v4 B3 | GCCTGCTCCATGGAACCCAAGA |
| PD HK3v4 FIP | TCAGAGCAACTCAGGGTTTCTTCCCCACTGTGGAAGCTCATGGAC |
| PD HK3v4 BIP | TCAGAGCTGGTGCAGGAGTGCGCTGGCTTGGATCTGCTGTAGC |
| PD HK3v4 FL | CCGCAACCCTGAAGACCCA |
| PD HK3v4 BL | GCAGTTCAAGGTGACAAGGGCAC |
| PD BATFv3 F3 | CTGAGTGTGAGAGCCCGGAAGATTT |
| PD BATFv3 B3 | TGTTCAGCACCGACGTGAAGTACTT |
| PD BATFv3 FIP | TACGATTTTTCTCCCTCCTCTGAACTCTTCAGCAGTGACTCCAGCTTCAGC |
| PD BATFv3 BIP | GAAGAGCCGACAGAGGCAGTGCTTGATCTCCTTGCGTAGAGCC |
| PD BATFv3 LF | CATCAGATGAGTCCTGTTTGCCAGG |
| PD BATFv3 LB | GCACCTGGAGAGCGAAGACCT |
| PE DEFA4 i2v4-12 F3 | AGGTGATGAGGCTCCAGG |
| PE DEFA4 i2v4-12 B3 | TGAAACTCACACCACCAATGA |
| PE DEFA4 i2v4-12 FIP | ACCTGAAGAGCAGAGCTTTTATCCCAGCGTGGGCCAGAAGAC |
| PE DEFA4 i2v4-12 BIP | TCAGGCTCAACAAGGGGCATGGCAGTTCCCAACACGAAGTT |
| PE DEFA4 i2v4-12 FL | GCTCTTGCAGATTAGTATTCTGCCGG |
| PE DEFA4 i2v4-12 BL | GTCCTGTATAGATAAAGGAAACGTA |
| PD LY86v9 F3 | CTTGACCTAGCTCTCATGTCTCAA |
| PD LY86v9 B3 | CACATGATAGTAGCATTGGCACA |
| PD LY86v9 FIP | GCATAGTAAATCTGCTCTCCTTTCCGGCTCATCTGTTTTGAATTTCTCCTA |
| PD LY86v9 BIP | GGCCTGTCAATAATCCTGAATTTACTGGTGGACCGTTTTTCAGTGTAC |
| PD LY86v9 FL | CCACAGAAAGAAAACTTGGGCA |
| PD LY86v9 BL | CCTCAGGGAGAATACCAGGTTT |
| PD TGFBlv4 F3 | GGTGATGAAATCCTGGTTAGCGGA |
| PD TGFBlv4 B3 | CGCTGATGCTTGTTTGAAGATCTC |
| PD TGFBlv4 FIP | AGGCTCCTTGTTGACACTCACCACGCCCTGGTGCGGCTAAAGTCT |
| PD TGFBlv4 BIP | TGACATCATGGCCACAAATGGCGTCAGAGTCTGCAAGTTCATCCCCT |
| PD TGFBlv4 LF | GCTGACTTCCAGCTTGTCACCT |
| PD TGFBlv4 LB | CTCCAGCCAACAGACCTCAGGAA |
| PE HLA-DPB1v1 F3 | CTGCGGAGTACTGGAACAG |
| PE HLA-DPB1v1 B3 | CGTCACGTGGCAGACAAG |
| PE HLA-DPB1v1 FIP | GCCCAGCTCGTAGTTGTGTCTGGAAGGACATCCTGGAGGAGA |
| PE HLA-DPB1v1 BIP | CCGAGTCCAGCCTAGGGTGAGGTTGTGGTGCTGCAAGG |
| PE HLA-DPB1v1-1 FL | ATCCTGTCCGGCACTGC |
| PE HLA-DPB1v1-1 BL | ATGTTTCCCCCTCCAAGAAGG |

TABLE 13
Multiple regression model in the COVID-19 cohort with
severe respiratory failure as the dependent variable.

| | Estimate | Std. Error | Statistic | P-value |
|---|---|---|---|---|
| (Intercept) | −13.5 | 4.36 | −3.10 | 0.00197 |
| 6-mRNA score | 5.42 | 4.04 | 1.34 | 0.181 |
| Age (years) | 0.104 | 0.0460 | 2.26 | 0.0239 |
| CRP (mg/l) | 0.0132 | 0.00782 | 1.70 | 0.090 |
| PCT (ng/ml) | −0.185 | 0.210 | −0.882 | 0.378 |
| Gender (Male) | −1.37 | 1.297 | −1.06 | 0.290 |
| SOFA | 0.73 | 0.301 | 2.42 | 0.016 |

### IX. REFERENCES

1. coronavirusjhu.edu/map.html. (Johns Hopkins University. 2020).
2. F. Zhou et al., Clinical course and risk factors for mortality of adult inpatients with COVID-19 in Wuhan, China: a retrospective cohort study. Lancet 395, 1054-1062 (2020).
3. D. Wang et al., Clinical Characteristics of 138 Hospitalized Patients With 2019 Novel Coronavirus-Infected Pneumonia in Wuhan, China. Jama. (2020).
4. M. Cevik, C. Bamford, A. Ho, COVID-19 pandemic—A focused review for clinicians. Clin Microbiol Infect, (2020).
5. C. i. C. f. D. C. a. P. Epidemiology Working Group for NCIP Epidemic Response, [The epidemiological characteristics of an outbreak of 2019 novel coronavirus diseases (COVID-19) in China]. Zhonghua Liu Xing Bing Xue Za Zhi 41, 145-151 (2020).
6. W. J. Guan et al., Clinical Characteristics of Coronavirus Disease 2019 in China. N Engl J Med 382, 1708-1720 (2020).
7. D. A. Berlin, R. M. Gulick, F. J. Martinez, Severe Covid-19. N Engl J Med, (2020).
8. W. Liang et al., Development and Validation of a Clinical Risk Score to Predict the Occurrence of Critical Illness in Hospitalized Patients With COVID-19. JAMA Intern Med, (2020).
9. P. Mehta et al., COVID-19: consider cytokine storm syndromes and immunosuppression. Lancet 395, 1033-1034 (2020).
10. G. Monteleone, P. C. Sarzi-Puttini, S. Ardizzone, Preventing COVID-19-induced pneumonia with anticytokine therapy. Lancet Rheumatol 2, e255-e256 (2020).
11. X. Xu et al., Effective treatment of severe COVID-19 patients with tocilizumab. Proc Natl Acad Sci USA, (2020).
12. F. Wang et al., The laboratory tests and host immunity of COVID-19 patients with different severity of illness. JCI Insight, (2020).
13. X. Zhang et al., Viral and host factors related to the clinical outcome of COVID-19. Nature, (2020).
14. L. Wynants et al., Prediction models for diagnosis and prognosis of covid-19 infection: systematic review and critical appraisal. BMJ 369, m1328 (2020).
15. T. E. Sweeney, A. Shidham, H. R. Wong, P. Khatri, A comprehensive time-course-based multicohort analysis of sepsis and sterile inflammation reveals a robust diagnostic gene set. Sci Transl Med 7, 287ra271 (2015).
16. M. Andres-Terre et al., Integrated, Multi-cohort Analysis Identifies Conserved Transcriptional Signatures across Multiple Respiratory Viruses. Immunity 43, 1199-1211 (2015).
17. T. E. Sweeney, H. R. Wong, P. Khatri, Robust classification of bacterial and viral infections via integrated host gene expression diagnostics. Sci Transl Med 8, 346ra391 (2016).
18. T. E. Sweeney et al., A community approach to mortality prediction in sepsis via gene expression analysis. Nat Commun 9, 694 (2018).
19. M. B. Mayhew et al., A generalizable 29-mRNA neural-network classifier for acute bacterial and viral infections. Nat Commun 11, 1177 (2020).

0 20. H. Zheng et al., Multi-cohort analysis of host immune response identifies conserved protective and detrimental modules associated with severity irrespective of virus, medRxiv, 2020.
21. M. B. Mayhew et al., Optimization of genomic classifiers for clinical deployment: evaluation of Bayesian optimization for identification of predictive models of acute infection and in-hospital mortality. ArXiv, 2003.12310 (2020).
22. D. Krstajic, L. J. Buturovic, D. E. Leahy, S. Thomas, Cross-validation pitfalls when selecting and assessing regression and classification models. J Cheminform 6, 10 (2014).
23. C. Ambroise, G. J. McLachlan, Selection bias in gene extraction on the basis of microarray gene-expression data Proc Natl Acad Sci USA 99, 6562-6566 (2002).
24. R. Almansa et al., Critical COPD respiratory illness is linked to increased transcriptomic activity of neutrophil proteases genes. BMC Res Notes 5, 401 (2012).
25. R. Almansa et al., Transcriptomic correlates of organ failure extent in sepsis. J Infect 70, 445-456 (2015).
26. C. A, van de Weg et al., Time since onset of disease and individual clinical markers associate with transcriptional changes in uncomplicated dengue. PLoS Negl Trop Dis 9, e0003522 (2015).
27. R Pankla et al., Genomic transcriptional profiling identifies a candidate blood biomarker signature for the diagnosis of septicemic melioidosis. Genome Biol 10. R127 (2009).
28. J. F. Bermejo-Martin et al., Host adaptive immunity deficiency in severe pandemic influenza. Crit Care 14, R167 (2010).
29. M. P. Berry et al., An interferon-inducible neutrophil-driven blood transcriptional signature in human tuberculosis. Nature 466, 973-977 (2010).
30. J. E. Berdal et al., Excessive innate immune response and mutant D222G/N in severe A (H1N1) pandemic influenza. J Infect 63, 308-316 (2011).
31. T. Dolinay et al., Inflammasome-regulated cytokines are critical mediators of acute lung injury. Am J Respir Crit Care Med 185, 1225-1234 (2012).
32. G. P. Parnell et al., A distinct influenza infection signature in the blood transcriptome of patients with severe community-acquired pneumonia. Crit Care 16, R157 (2012).
33. G. P. Pamell et al., Identifying key regulatory genes in the whole blood of septic patients to monitor underlying immune dysfunctions. Shock 40, 166-174 (2013).
34. M. Kwissa et al., Dengue virus infection induces expansion of a CD14(+)CD16(+) monocyte population that stimulates plasmablast differentiation. Cell Host Microbe 16, 115-127 (2014).
35. N. M. Suarez et al., Superiority of transcriptional profiling over procalcitonin for distinguishing bacterial from viral lower respiratory tract infections in hospitalized adults. J Infect Dis 212, 213-222 (2015).
36. B. P. Scicluna et al., A molecular biomarker to diagnose community-acquired pneumonia on intensive care unit admission. Am J Respir Crit Care Med 192, 826-835 (2015).
37. Y. Zhai et al., Host Transcriptional Response to Influenza and Other Acute Respiratory Viral Infections—A Prospective Cohort Study. PLoS Pathog 11, e1004869 (2015).
38. B. M. Tang et al., A novel immune biomarker. Eur Respir J 49, (2017).
39. F. Venet et al., Modulation of LILRB2 protein and mRNA expressions in septic shock patients and after ex vivo lipopolysaccharide stimulation. Hum Immunol 78, 441-450 (2017).
40. T. E. Sweeney, W. A. Haynes, F. Vallania, J. P. Ioannidis, P. Khatri, Methods to increase reproducibility in differential gene expression via meta-analysis. Nucleic Acids Res (2016).
41. W. A. Haynes et al., Empowering Multi-Cohort Gene Expression Analysis to Increase Reproducibility. Pac Symp Biocomput 22, 144-153 (2017).
42. F. Vallania et al., Leveraging heterogeneity across multiple datasets increases cell-mixture deconvolution accuracy and reduces biological and technical biases. Nat Commun 9, 1-8 (2018).
43. M. Robinson et al., A 20-Gene Set Predictive of Progression to Severe Dengue. Cell Rep 26, 1104-11 l.e 1104 (2019).
44. L. Fagerberg et al., Analysis of the human tissue-specific expression by genome-wide integration of transcriptomics and antibody-based proteomics. Mol Cell Proteomics 13, 397-406 (2014).

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”. “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

All patents, patent applications, publications, and descriptions mentioned herein are incorporated by reference in their entirety for all purposes. None is admitted to be prior art. Where a conflict exists between the instant application and a reference provided herein, the instant application shall dominate.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list: for example “1, 2 and/or 3” is equivalent to “‘1’ or ‘2’ or ‘3’ or ‘1 and 2’ or ‘1 and 3’ or ‘2 and 3’ or ‘1, 2 and 3’”. Whenever a range is given in the specification, for example, a temperature range, a time range, or a composition range, all intermediate ranges and subranges, as well as all individual values included in the ranges given are intended to be included in the disclosure.

1. A method of informing urgent care decisions for a subject in an emergency room or other clinical facility, the subject having a diagnosis of a viral infection, the method comprising:
(i) receiving a biological sample that was obtained from the subject;
(ii) detecting expression levels of TGFBI, DEFA4, LY86, BATF and HK3 biomarkers in the biological sample; and
(iii) determining a risk score based on the biomarker expression levels detected in step (ii), the score corresponding to a risk of mortality or of a need for ICU care of the subject over a specified length of time.

2. (canceled)

3. The method of claim 1, wherein the specified length of time is 30 days.

4. The method of claim 1, further comprising detecting the level of expression of an HLA-DPB1 biomarker in the biological sample in step (ii).

5. The method of claim 1, comprising comparing the score to one or more thresholds corresponding to one or more discrete levels of risk of need for ICU care or mortality over 30 days.

6. The method of claim 5, wherein the score is compared to two thresholds that define a (i) low, (ii) intermediate, and (iii) high risk of need for ICU care or mortality over 30 days, allowing the subject to be classified into one of three risk categories corresponding to each level (i-iii) of risk.

7. The method of claim 1, wherein the risk score is also based on one or more clinical parameters determined for the subject.

8. The method of claim 7, wherein the one or more clinical parameters comprises age or a clinical risk score.

9. The method of claim 8, wherein the clinical risk score is a sequential organ failure assessment (SOFA) score.

10. The method of claim 1, wherein the expression of the biomarkers is detected using qRT-PCR or isothermal amplification.

11. The method of claim 10, wherein the isothermal amplification is qRT-LAMP.

12. (canceled)

13. The method of claim 1, wherein the biological sample is a blood sample.

14. The method of claim 1, wherein the diagnosis is based on a detection of viral antigen or viral nucleic acid in a biological sample taken from the subject.

15. The method of claim 1, wherein the diagnosis is based on a detection of the expression levels of host biomarkers associated with viral infection in a biological sample taken from the subject.

16. The method of claim 1, wherein the expression levels of the biomarkers are detected within 24 hours of the diagnosis of viral infection.

17. The method of claim 6, wherein the threshold for a determination of a low risk of mortality or a need for ICU care over 30 days corresponds to a likelihood ratio of less than 0.15.

18. The method of claim 6, wherein the threshold for a determination of an intermediate risk of need for ICU care or mortality over 30 days corresponds to a likelihood ratio of from 0.15 to 5.

19. (canceled)

20. (canceled)

21. The method of claim 1, wherein the urgent care associated with said urgent care decisions comprises administering organ-supportive therapy, administering a therapeutic drug, admitting the subject to an ICU, or administering a blood product.

22. The method of claim 21, wherein the subject has been classified as having an intermediate (ii) or high (iii) risk of need for ICU care or mortality over 30 days.

23. The method of claim 22, wherein the subject has been classified as having a high (iii) risk of 30-day mortality.

24. (canceled)

25. (canceled)

26. The method of claim 1, wherein the viral infection is an influenza or SARS-CoV-2 infection.

27. (canceled)

28. A test kit for detecting the expression levels of five or more biomarkers in a subject with a viral infection, wherein the kit comprises reagents for specifically detecting the expression levels of the five or more biomarkers, and wherein the biomarkers comprise TGFBI, DEFA4, LY86, BATF and HK3.

29-39. (canceled)


SEQUENCE LISTING (36 sequences)

SEQ ID NO: 1 - DNA, 24 bp, Artificial Sequence
  Description of Artificial Sequence Synthetic primer
acctgaggagagtgactagcttct

SEQ ID NO: 2 - DNA, 22 bp, Artificial Sequence
  Description of Artificial Sequence Synthetic primer
gcctgctccatggaacccaaga

SEQ ID NO: 3 - DNA, 45 bp, Artificial Sequence
  Description of Artificial Sequence Synthetic primer
tcagagcaactcagggtttcttccccactgtggaagctcatggac

SEQ ID NO: 4 - DNA, 43 bp, Artificial Sequence
  Description of Artificial Sequence Synthetic primer
tcagagctggtgcaggagtgcgctggcttggatctgctgtagc

SEQ ID NO: 5 - DNA, 19 bp, Artificial Sequence
  Description of Artificial Sequence Synthetic primer
ccgcaaccctgaagaccca

SEQ ID NO: 6 - DNA, 23 bp, Artificial Sequence
  Description of Artificial Sequence Synthetic primer
gcagttcaaggtgacaagggcac

SEQ ID NO: 7 - DNA, 25 bp, Artificial Sequence
  Description of Artificial Sequence Synthetic primer
ctgagtgtgagagcccggaagattt

SEQ ID NO: 8 - DNA, 25 bp, Artificial Sequence
  Description of Artificial Sequence Synthetic primer
tgttcagcaccgacgtgaagtactt

SEQ ID NO: 9 - DNA, 51 bp, Artificial Sequence
  Description of Artificial Sequence Synthetic primer
tacgatttttctccctcctctgaactcttcagcagtgactccagcttcagc

SEQ ID NO: 10 - DNA, 43 bp, Artificial Sequence
  Description of Artificial Sequence Synthetic primer
gaagagccgacagaggcagtgcttgatctccttgcgtagagcc

SEQ ID NO: 11 - DNA, 25 bp, Artificial Sequence
  Description of Artificial Sequence Synthetic primer
catcagatgagtcctgtttgccagg

SEQ ID NO: 12 - DNA, 21 bp, Artificial Sequence
  Description of Artificial Sequence Synthetic primer
gcacctggagagcgaagacct

SEQ ID NO: 13 - DNA, 18 bp, Artificial Sequence
  Description of Artificial Sequence Synthetic primer
aggtgatgaggctccagg

SEQ ID NO: 14 - DNA, 21 bp, Artificial Sequence
  Description of Artificial Sequence Synthetic primer
tgaaactcacaccaccaatga

SEQ ID NO: 15 - DNA, 42 bp, Artificial Sequence
  Description of Artificial Sequence Synthetic primer
acctgaagagcagagcttttatcccagcgtgggccagaagac

SEQ ID NO: 16 - DNA, 41 bp, Artificial Sequence
  Description of Artificial Sequence Synthetic primer
tcaggctcaacaaggggcatggcagttcccaacacgaagtt

SEQ ID NO: 17 - DNA, 26 bp, Artificial Sequence
  Description of Artificial Sequence Synthetic primer
gctcttgcagattagtattctgccgg

SEQ ID NO: 18 - DNA, 25 bp, Artificial Sequence
  Description of Artificial Sequence Synthetic primer
gtcctgtatagataaaggaaacgta

SEQ ID NO: 19 - DNA, 24 bp, Artificial Sequence
  Description of Artificial Sequence Synthetic primer
cttgacctagctctcatgtctcaa

SEQ ID NO: 20 - DNA, 23 bp, Artificial Sequence
  Description of Artificial Sequence Synthetic primer
cacatgatagtagcattggcaca

SEQ ID NO: 21 - DNA, 51 bp, Artificial Sequence
  Description of Artificial Sequence Synthetic primer
gcatagtaaatctgctctcctttccggctcatctgttttgaatttctccta

SEQ ID NO: 22 - DNA, 48 bp, Artificial Sequence
  Description of Artificial Sequence Synthetic primer
ggcctgtcaataatcctgaatttactggtggaccgtttttcagtgtac

SEQ ID NO: 23 - DNA, 22 bp, Artificial Sequence
  Description of Artificial Sequence Synthetic primer
ccacagaaagaaaacttgggca

SEQ ID NO: 24 - DNA, 22 bp, Artificial Sequence
  Description of Artificial Sequence Synthetic primer
cctcagggagaataccaggttt

SEQ ID NO: 25 - DNA, 24 bp, Artificial Sequence
  Description of Artificial Sequence Synthetic primer
ggtgatgaaatcctggttagcgga

SEQ ID NO: 26 - DNA, 24 bp, Artificial Sequence
  Description of Artificial Sequence Synthetic primer
cgctgatgcttgtttgaagatctc

SEQ ID NO: 27 - DNA, 45 bp, Artificial Sequence
  Description of Artificial Sequence Synthetic primer
aggctccttgttgacactcaccacgccctggtgcggctaaagtct

SEQ ID NO: 28 - DNA, 47 bp, Artificial Sequence
  Description of Artificial Sequence Synthetic primer
tgacatcatggccacaaatggcgtcagagtctgcaagttcatcccct

SEQ ID NO: 29 - DNA, 22 bp, Artificial Sequence
  Description of Artificial Sequence Synthetic primer
gctgacttccagcttgtcacct

SEQ ID NO: 30 - DNA, 23 bp, Artificial Sequence
  Description of Artificial Sequence Synthetic primer
ctccagccaacagacctcaggaa

SEQ ID NO: 31 - DNA, 19 bp, Artificial Sequence
  Description of Artificial Sequence Synthetic primer
ctgcggagtactggaacag

SEQ ID NO: 32 - DNA, 18 bp, Artificial Sequence
  Description of Artificial Sequence Synthetic primer
cgtcacgtggcagacaag

SEQ ID NO: 33 - DNA, 42 bp, Artificial Sequence
  Description of Artificial Sequence Synthetic primer
gcccagctcgtagttgtgtctggaaggacatcctggaggaga

SEQ ID NO: 34 - DNA, 38 bp, Artificial Sequence
  Description of Artificial Sequence Synthetic primer
ccgagtccagcctagggtgaggttgtggtgctgcaagg

SEQ ID NO: 35 - DNA, 17 bp, Artificial Sequence
  Description of Artificial Sequence Synthetic primer
atcctgtccggcactgc

SEQ ID NO: 36 - DNA, 21 bp, Artificial Sequence
  Description of Artificial Sequence Synthetic primer
atgtttccccctccaagaagg